(19) United States
(12) Patent Application Publication
(10) Pub. No.: US 2013/0210024 A1
(43) Pub. Date: Aug. 15, 2013

(54) COMBINATION TREATMENT OF CANCER

(71) Applicants: Qiang Yu, Singapore (SG); Zhenlong Wu, Singapore (SG)

(72) Inventors: Qiang Yu, Singapore (SG); Zhenlong Wu, Singapore (SG)

(73) Assignee: Agency for Science, Technology and Research, Singapore (SG)

(21) Appl. No.: 13/879,494

(22) PCT Filed: Oct. 14, 2011
(86) PCT No.: PCT/SG2011/000358

§ 371 Date: Apr. 15, 2013

(30) Foreign Application Priority Data
Oct. 15, 2010 (SG) 201007581-0

(51) Int. Cl. C12N 9/00

(52) U.S. Cl. CPC C12N 9/93
USPC 435/6.18; 435/375; 536/6.4; 536/18.1; 536/23.1; 536/27.13; 548/304.7; 568/308

(57) ABSTRACT

The present technology relates to a method of treating cancer by sensitizing human tumours to DNA damaging therapies through activating FBXO32 expression. Transactivation of FBXO32 through the inhibition of EZH2, a histone methyltransferase, decreases p21 protein induction which results in the sensitization of human tumours to chemotherapy. The method further provides a prognostic method to determine if a combination treatment would be effective.

### CROSS-REFERENCE TO RELATED APPLICATION

This application claims the benefit of Singapore Patent Application No. 201007581-0 filed on 15 Oct. 2010, the entire contents of which are incorporated herein by reference.

### FIELD OF THE INVENTION

The present invention relates a method of treating cancer by sensitizing human tumours to DNA damaging therapies and compounds used in treating cancer.

### BACKGROUND ART

Cancer is one of the main diseases of current times causing 13% of all deaths globally. New aspects of the genetics of cancer pathogenesis, such as DNA methylation are increasingly recognized as important. Most of the standard chemotherapy compounds induce DNA damage to activate cell cycle checkpoint for DNA repair. Most chemotherapy compounds are designed to induce cell cycle arrest or to induce apoptosis when DNA repair process fails.

P21 is a major regulator of cell cycle progression at G1 and S phase. As a cell cycle checkpoint activator having an important role in the stress response, p21 has been reported to induce cell cycle arrest and prevent apoptosis in response to DNA damaging agents such as radiation or chemotherapy. This stress response of p21 can actually hinder cancer treatments that target rapidly dividing cells to damage the DNA of the cells and induce apoptosis. The expression of p21 protein has been reported to increase in primary acute myeloid leukemia (AML) cells when treated with a polycomb repressive complex inhibitor.

Both p21 and Chk1 are known to play a role in protecting DNA damage-induced apoptosis (Bunz et al., 1999; Kawabe, 2004; Li et al., 2007; Rodriguez & Meuth, 2006; Weiss, 2003). Following DNA damage, mammalian cells activate cell cycle checkpoint mechanisms to induce cell cycle arrest and protect cells from apoptosis (Zhou & Elledge, 2000). The interconnections between the pathways regulating the cell cycle checkpoints and apoptosis dictates the cellular outcome to DNA damage (Sancar et al., 2004), but whether these pathways are differentially regulated by oncogenic lesions in tumor cells to allow cancer specific perturbation is poorly understood. Although inhibition of key cell cycle checkpoint regulators such as cyclin-dependent kinase inhibitor p21 and Chk1/2 have been shown to increase the sensitivity to DNA damage in p53-proficient and p53-deficient cancer cells, respectively (Beuvink et al., 2005; Bunz et al., 1999; Fan et al., 1997; Graves et al., 2000; Jascur et al., 2005; Kawabe, 2004; Li et al., 2007; Mukhopadhyay et al., 2005; Seoane et al., 2002; Tse & Schwartz, 2004; Weiss, 2003; Wouters et al., 1997), a treatment strategy to simuteniously abrogate both G1 and G2/M checkpoint and thus sensitizes both p53 wild-type and mutant tumors is yet to be developed.

p21 has been suggested to be critical in determining cellular sensitivity to DNA-damaging agents, as p21-deficient cells are defective in the cell cycle checkpoint and are highly sensitive to DNA damage (Bunz et al., 2002; Bunz et al., 1999; Fan et al., 1997; Waldman et al., 1996; Wouters et al., 1997). Inhibition of p21 transcription through overexpression of Myc (Seoane et al., 2002) or inhibition of p21 protein translation by the mTOR inhibitor RAD001 can lead to the conversion of DNA damage-induced p53 response from growth arrest to apoptosis (Beuvink et al., 2005).

Polycomb protein EZH2 is a histone methyltransferase that is frequently over-expressed in a wide variety of human malignancies (Bracken & Helin, 2009; Simon & Lange, 2008) and is implicated in cell proliferation, invasion and metastasis (Bracken et al., 2003; Cao et al., 2008; Cao & Zhang, 2004; Chen et al., 2005; Fujii et al., 2008; Kleer et al., 2003; Min et al., 2010; Varambally et al., 2002; Yang et al., 2009). Mechanistically, the oncogenic function of EZH2 has been attributed to an associated histone H3 lysine 27 trimethylation (H3K27Me3), leading to transcriptional repression of tumor suppressor genes, including InK4b-Arf-InK4A (Bracken et al., 2007), E-cadherin (Cao et al., 2008), adrenergic receptor β2 (Yu et al., 2007), RUNX3 (Fujii et al., 2008), p57 (also called CDKN1C) (Yang et al., 2009), Bim (Wu et al., 2009), and DAB2IP (Chen et al., 2005; Min et al., 2010). It is not clear, whether EZH2 overexpression in cancer cells plays a role in affecting cellular outcome to DNA damage.

The FBXO32 gene encodes the protein 32 which is a member of the F-box protein family characterized by an F-box motif of approximately 40 amino acids. The F-box proteins constitute one of the four subunits of the ubiquitin protein ligase complex called SCFs (SKP1-cullin-F-box), which function in phosphorylation-dependent ubiquitination. The protein encoded by this gene belongs to the Fbxs class, containing either different protein-protein interaction modules and contains an F-box domain. This protein is highly expressed during muscle atrophy, whereas mice deficient in this gene were found to be resistant to atrophy. FBXO32 expression is downregulated in multiple types of cancer, like breast cancer, colon cancer, and AML. The expression of protein 32 has been reported to increase in primary acute myeloid leukemia (AML) cells when treated with a polycomb repressive complex inhibitor.

### SUMMARY OF THE INVENTION

The present invention seeks to ameliorate at least some of the difficulties discussed above. This may be useful in treating or slowing cancer cells to ameliorate some of the difficulties with the current treatment of cancer. Aspects of the invention further seeks to provide in vivo and in vitro methods, for inducing apoptosis or prognosing suitable treatments.

Accordingly the first aspect of the invention is a method of inducing apoptosis of a cell comprising the steps of: increasing expression of an FBXO32 polypeptide and decreasing expression of a p21 polypetide.

Preferably the expression of FBXO32 is increased by applying an inhibitor of a Polycomb protein histone methyltranserase (EZH2) expression of SEQ ID NO. 3 to the cell resulting in the decrease of p21 expression.

Preferably the method further provides the step of adding a chemotherapeutic agent to the cell.

A further aspect of the invention is a compound comprising a composition capable of increasing expression of FBXO32 polypeptide and decreasing expression of p21 polypeptide and a DNA damaging agent.

Preferably the expression of FBXO32 is increased by applying an inhibitor of a Polycomb protein histone methyltranserase (EZH2) expression of SEQ ID NO. 3 to the cell resulting in the decrease of p21 expression.

Preferably the DNA damaging agent is a chemotherapeutic agent.

A further aspect of the invention comprises a method of predicting the effectiveness of compound of the invention comprising the step of determining a first expression profile of FBXO32 in a subject who is not diagnosed with cancer; determining a second expression profile of FBXO32 in a subject who is diagnosed with cancer and comparing the first and second expression profile whereby when the second expression profile is less than the first expression profile the subject who is diagnosed with cancer will benefit from treatment with the compound of the invention.

Throughout this document, unless otherwise indicated to the contrary, the terms “comprising”, “consisting of”, and the like, are to be construed as non-exhaustive, or in other words, as meaning “including, but not limited to”.

### BRIEF DESCRIPTION OF THE DRAWINGS

The invention will be better understood by reference to the following drawings in which:

FIG. 1 EZH2 Depletion Promotes DNA Damage-Induced Apoptosis by Abrogating Cell Cycle Checkpoints

A, FACS analysis showing that EZH2 depletion abrogates ADR (1 μM) or ETO- (10 μM) induced G1 arrest (24 h) and promotes apoptosis in U2OS cells (48 h). B, Immunoblot analysis showing EZH2 depletion abolished ADR or ETO-induced p21 accumulation and Chk1 phosphorylation in U2OS cells. C, EZH2 depletion abrogates ADR-induced G2 arrest and promotes apoptosis in p53-deficient Saos-2 cells. D, Immuoblot analysis showing EZH2 depletion blocked ADR-induced Chk1 phosphorylation in Saos-2 cells. E, EZH2 depletion does not affect ADR-induced p21mRNA induction. F, p21 protein levels in U2OS cells transfected with NC or EZH2 siRNA, followed by ADR treatment in the absence or presence of proteasome inhibitors MG132 or MG115. G, Defective induction of p21 and p-Chk1 by ADR upon EZH2 depletion is rescued by wild-type EZH2 but not EZH2Δ. H, FACS analysis in U2OS showing the effects of EZH2 depletion on checkpoints abrogation and apoptosis is rescued by wild-type EZH2, but no by EZH2Δ.

FIG. 2 EZH2 Repress FBXO32 Expression in Multiple Cancer Cells

A, qRT-PCR analysis showing the identification of FBXO32 mRNA, but not FBXO31mRNA, are up-regulated following EZH2 depletion in U2OS, HCT116 and MCF-7 cells. B, immunoblots showing that EZH2 knockdown up-regulates FBXO32 protein levels in multiple cancer cell lines. C, Box-plots showing the expression levels of EZH2 mRNA (left panel) and FBXO32 mRNA (right panel) in 24 pairs of patient-derived primary colon tumor samples and matched normal controls. D, ChIP-PCR detection of EZH2 and H3K27me3 at FBXO32 locus in HCT116 cells. E ChIP-PCR showing EZH2 and H3K27me3 enrichment of FBXO32 in U2OS, MCF7 and MCF10A cells. F Expression levels of EZH2 and FBXO32 in MCF7 and MCF10A cells.

FIG. 3 Induction of FBXO32 Following EZH2 Depletion is Functionally Required for p21 Degradation and Increased Apoptosis During DNA Damage.

A, Western blot showing FBXO32 induction following EZH2 depletion is required for p21 degradation in ADR-treated U2OS cells. B, FACS analysis showing that FBXO32 induction is required for ETO-induced apoptosis following EZH2 knockdown in U2OS cells. C Immuno-blot analysis of the effect of shRNA-mediated knockdown of FBXO32 on p21 degradation following EZH2 depletion in ADR-treated HCT116 cells. D, FACS analysis showing FBXO32 is required for ADR-induced apoptosis following EZH2 depletion in HCT116 cells. E, western blot showing the requirement of FBXO32 for p21 degradation following EZH2 depletion in MCF-7 cells. F, p21 levels in HCT116 cells transduced with a retrovirus expressing FBXO32 or empty vector followed by ADR or ETO treatment. G, FACS analysis showing ETO-induced G1 arrest in mitotic synchronized HCT116 cells expressing FBXO32 or empty vector. H, FACS analysis showing ADR or ETO-induced apoptosis in FBXO32 or empty vector-expressed HCT116 and HCT116 shRNA p21.

FIG. 4 Pharmacologic Depletion of EZH2 Phenocopies EZH2 Knockdown in Modulating DNA Damage Response

A, FACS analysis showing combination of DZNep with ADR or ETO abrogates checkpoints activation in HCT116 or U2OS cells. B, combination of DZNep with ADR or ETO abolishes ADR or ETO-induced p21 accumulation and Chk1 phosphorylation in HCT116 and U2OS cells. C, FACS analysis showing the combination of DZNep with ADR or ETO abrogates checkpoints in p53-deficient HCT116 or Saos-2 cells. D, combination of DZNep with ADR or ETO abolishes DNA damage induced p21 accumulation and Chk1 phosphorylation in p53-deficient HCT116 and Saos-2 cells. E, FACS analysis showing FBXO32 is required for combination treatment-induced apoptosis in HCT116 cells. F, FBXO32 is functionally required for abrogation of p21 accumulation and Chk1 phosphorylation following DZNep and ETO combination treatment.

### DETAILED DISCLOSURE

Polycomb protein histone methyltranserase EZH2 is frequently overexpressed in human malignancy and is emerging as important in tumorigenesis. However, it is largely unknown whether EZH2 has a role in cancer cell life and death decision in response to genotoxic stress. Here we show that EZH2-mediated gene repression plays a role in modulating DNA damage response. EZH2 depletion results in abrogation of cell cycle checkpoints, directing DNA damage response towards predominant apoptosis in both p53-proficient and p53-deficient cancer cells. Mechanistically, EZH2 regulates DNA damage response, at least in part, through transcriptional repression of FBXO32, which directs p21 for proteasome-mediated degradation. Furthermore, pharmacological depletion of EZH2 by EZH2 knockdown demonstrates effects on cancer cell cycle checkpoints/apoptosis in a FBXO32-dependent manner. These data unravel a crucial role of EZH2 in determining the cancer cell outcome following DNA damage and suggest that inhibition of oncogenic EZH2 might serve as a potent strategy for improving conventional chemotherapy in a given malignancy.

The present technology relates to a method of inducing apoptosis of a cell comprising the steps of: increasing expression of an FBXO32 polypeptide and thereby decreasing expression of a p21 polypetide. The method is suitable for treating cancer by sensitizing human tumours to DNA damaging therapies through activating FBXO32 expression. The FBXO32 gene sequence described herein including that set out in SEQ ID No. 1. The FBXO32 gene sequence and SEQ ID No. 1 includes functional derivatives, homologues and variants that express a functional protein 32 as set out in SEQ ID No 2. We have found that transactivation of FBXO32 through the inhibition of EZH2, a histone methyltransferase, decreases p21 protein induction which results in the sensitization of human tumours to chemotherapy. There is provided method of inducing apoptosis of a cell comprising the steps of: applying an EZH2 inhibitor to the cell to increase expression of FBXO32 and decrease expression of p21. Preferably the method further provides the step of adding a DNA damaging agent to the cell. In one embodiment the DNA damaging agent is a chemotherapeutic agent.

An increase in FBXO32 expression is preferably defined as a 2 to 5 fold increase in the amount of FBXO32 polypeptide measured in a cancer cell after treatment when compared to the amount of FBXO32 polypeptide measured in a cancer cell before treatment. More preferably the increase may be a 3 to 5 fold increase or a 4 to 5 fold increase or most preferably a 4 fold increase in the amount of FBXO32 polypeptide measured in a cancer cell after treatment when compared to the amount of FBXO32 polypeptide measured in a cancer cell before treatment.

A decrease in p21 expression is preferably defined as a 3 to 5 fold decrease in the amount of p21 polypeptide measured in a cancer cell after treatment when compared to the amount of p21 polypeptide measured in a cancer cell before treatment.

A further aspect of the invention comprises a compound comprising a composition capable of increasing expression of FBXO32 polypeptide and decreasing expression of p21 polypeptide and a DNA damaging agent. The composition capable of increasing expression of FBXO32 polypeptide may be an EZH2 inhibitor. Wherein the EZH2 inhibitor and DNA damaging agent is as defined throughout the description. In an alternative embodiment the composition capable of increasing expression of FBXO32 polypeptide may be an expression vector capable of ectopic expression of FBXO32 polypeptide of SEQ ID NO. 2.

A further aspect of the invention comprises a method of predicting the effectiveness of compound of the invention comprising the steps of:
a) determining a first expression profile of FBXO32 in a subject who is not diagnosed with cancer;
b) determining a second expression profile of FBXO32 in a subject who is diagnosed with cancer; and
c) comparing the first and second expression profile whereby when the second expression profile is less than the first expression profile the subject who is diagnosed with cancer will benefit from treatment with the compound described herein.

A further aspect of the invention comprises a kit to determine an expression profile of FBXO32 in vitro comprising a reagent capable of binding selectively an FBXO32 polynucleotide of SEQ ID No. 1 or an FBXO32 polypeptide of SEQ ID NO. 2. The kit may further comprising a detection reagent capable of binding selectively a p21 polynucleotide of SEQ ID NO. 5 or a p21 polypeptide of SEQ ID NO. 6.

EZH2 is one of 3 core proteins of a polycomb repressive complex. EZH2 has a suppressor of variegation enhancer of zeste-trithorax (SET) domain. The EZH2 gene sequence described herein including that set out in SEQ ID No. 3. The EZH2 gene sequence and SEQ ID No. 3 includes functional derivatives, homologues and variants that express a functional EZH2 protein as set out in SEQ ID No 4.

A further aspect of the invention is a compound comprising an EZH2 inhibitor capable of increasing expression of FBXO32 and decreasing expression of p21 and a DNA damaging agent. Preferably the DNA damaging agent is a chemotherapeutic agent. The p21 gene sequence described herein including that set out in SEQ ID No. 5. The p21 gene sequence and SEQ ID No. 5 includes functional derivatives, homologues and variants that express a functional p21 protein as set out in SEQ ID No 6.

A further aspect of the invention comprises a method of predicting the effectiveness of compound of the invention comprising the step of determining a first expression profile of FBXO32 in a subject who is not diagnosed with cancer; determining a second expression profile of FBXO32 in a subject who is diagnosed with cancer and comparing the first and second expression profile whereby when the second expression profile is less than the first expression profile the subject who is diagnosed with cancer will benefit from treatment with the compound of the invention.

EZH2 Inhibitors

An EZH2 inhibitor is any protein, peptide, nucleic acid, such as siRNA, small molecule, compound or the like that can stop, hinder or block the expression of EZH2 protein. Alternatively an EZH2 inhibitor is any protein, peptide, nucleic acid, such as siRNA, small molecule, compound or the like that can stop, hinder or block the interaction of an EZH2 protein with the FBXO32 gene. Preferably the EZH2 inhibitor will stop, hinder or block the interaction of an EZH2 protein with the FBXO32 gene by interaction with the SET domain as set out in SEQ ID NO. 7. The SET domain of EZH2 is important for histone methylation and gene repression activity.

The EZH2 inhibitors provide the advantage of selectively sensitizing cancer cells with minimum effect on normal cells. Further EZH2 inhibitors may enhance the efficacy of DNA damaging agents allowing patients to be administered with less DNA damaging agents than currently required thereby reducing the toxic side effects.

Micro RNA

In some embodiments, the present invention provides MicroRNAs that inhibit the expression of EZH2. MicroRNAs are regulatory, non-protein-coding, endogenous RNAs that have recently gained considerable attention in the scientific community. They are 18-24 nucleotides in length and are thought to regulate gene expression through translational repression by binding to a target. They are also proposed to regulate gene expression by mRNA cleavage, and mRNA decay initiated by miRNA-guided rapid deadenylation. miRNAs are abundant, highly conserved molecules and predicted to regulate a large number of transcripts. To date the international miRNA Registry database has more than 600 human identified microRNAs and their total number in humans has been predicted to be as high as 1,000. Many of these microRNAs exhibit tissue-specific expression and many are defined to play a crucial role in variety of cellular processes such as cell cycle control, apoptosis, and haematopoiesis.

EZH2 expression is inhibited by miR-101 (SEQ ID NO. 8: UGCCCUGGCUCAGUUAUCACAGUGCUGAUGCUGUCUAUUCUAAAGGUACAG UACUGUGAUAACUGAAGGAUGGCA). Another siRNAs that was found to be effective is described herein including that set out in SEQ ID No. 9 and 10. Accordingly, in some embodiments, the present invention provides methods of inhibiting EZH2 expression and/or activity using microRNAs (e.g., miR-101). In some embodiments, miRNAs inhibit the expression of EZH2 protein. In other embodiments, miRNAs inhibit EZH2 activity.

The present invention is not limited to miR-101. Additional miRNAs can be screened for their activity against EZH2 using any suitable method, including, but not limited to, those disclosed below. Suitable nucleic acids for use in the methods described herein include, but are not limited to, pri-miRNA, pre-miRNA, mature miRNA or fragments of variants thereof that retain the biological activity of the miRNA and DNA encoding a pri-miRNA, pre-miRNA, mature miRNA, fragments or variants thereof, or DNA encoding regulatory elements of the miRNA.

In some embodiments the nucleic acid encoding the disclosed inhibitory nucleic acids, for example a miRNA molecule, is on a vector. These vectors include a sequence encoding a mature microRNA and in vivo expression elements. In a preferred embodiment, these vectors include a sequence encoding a pre-miRNA and in vivo expression elements such that the pre-miRNA is expressed and processed in vivo into a mature miRNA. In other embodiments, these vectors include a sequence encoding the pri-miRNA gene and in vivo expression elements. In this embodiment, the primary transcript is first processed to produce the stem-loop precursor miRNA molecule. The stem-loop precursor is then processed to produce the mature microRNA. Vectors include, but are not limited to, plasmids, cosmids, phagemids, viruses, other vehicles derived from viral or bacterial sources that have been manipulated by the insertion or incorporation of the nucleic acid sequences for producing the microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One of skill in the art can readily employ other vectors known in the art.

Small Molecule Therapies

In other embodiments, the present invention provides small molecule inhibitors of EZH2 expression or activity. Isoliquiritigenin is an inhibitor of EZH2 expression. Accordingly, in some embodiments, the present invention provides methods of treating cancer using isoliquiritigenin or related compounds.

Isoliquiritigenin, one of the components in the root of Glycyrrhiza glabra L., is a member of the flavonoids. Other flavonoids are known to have an anti-tumor activity in vitro and in vivo. Isoliquiritigenin has also been shown to be a soluble guanylate cyclase activator and to possess estrogen-like activity. Isoliquiritigenin has been shown to activate estrogen receptor-alpha and -beta and trigger biochemical reactions in cancer cells. The COX-2 inhibitory activity of isoliquiritigenin has also been demonstrated. As used herein, isoliquiritigenin refers to CAS Reg. No. 961-29-5; also known as 2′,J,d′-trihydroxychalcone, a pharmaceutically acceptable salt or ester of isoliquiritigenin, a selectively-substituted analog of isoliquiritigenin, an extract of Glycyrrhiza uralersis 5 or Glycyrrhiza glabra, or a combination comprising one or more of the foregoing compounds. An ester of isoliquiritigenin is preferably a glycoside of isoliquiritigenin.

There is no particular limit on the monosacharide or polysaccharide used to form the glycoside of isoliquiritigenin. Suitable monosaccharide sugars include, for example, glucose, glucuronic acid, mannose, fructose, galactose, xylose, rutinose, rhamnose, and the like, and combinations comprising one or more of the foregoing monosaccharides. Suitable polysaccharides include, for example, dimers, trimers, oligomers, and polymers formed from one or more of the above monosaccharides.

An isoliquiritigenin analog includes, for example, phloretin, 2′,4,4′ trihydroxychalcone, or the like, or a combination comprising one or more of the foregoing isoliquiritigenin analogs.

Methods for synthesizing or isolating isoliquiritigenin, its pharmaceutically acceptable salts or esters, its selectively substituted analogs, are known in the art. See, for example, S. K. Srivastava et al., Indian J. Chem., Sect. B (1981), 20B(4): 347-8; Macias et al., Phytochemistry (1998), 50(1): 35-46, each of which is herein incorporated by reference.

In some embodiments, when isoliquiritigenin is present, the isoliquiritigenin comprises greater than or equal to 0.5 percent of the total weight, more preferably greater than or equal to about 1 percent of the total weight, still more preferably greater than or equal to about 2 percent of the total weight, even more preferably greater than or equal to about 5 percent of the total weight, even more preferably greater than or equal to about 10 percent of the total weight, still more preferably greater than or equal to about 20 percent of the total weight of the composition.

In some embodiments, the cancer is colorectal cancer, osteosarcoma or breast cancer. In other embodiments, the cancer is bladder, prostate, or other solid tumors. Additional small molecule EZH2 inhibitors are identified, for example, using the compositions and methods of the present invention. The present invention additionally contemplates mimetics, analogs and modified forms of isoliquiritigenin.

Additional small molecule inhibitors include S-adenosylhomocysteine hydrolase inhibitor 3-Deazaneplanocin A (DZNep). In some embodiments, these compounds find use in the inhibition of EZH2, alone or in combination with additional therapeutic agents described herein.

Preferably DZNep has the structure of formula I:

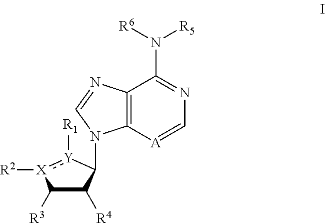

wherein:

X and Y are independently C or O;

### A is C or N;

[symbol] is a single bond or a double bond;

R¹ and R² are independently either absent or selected from the group consisting of hydrogen, halogen, optionally substituted alkyl, optionally substituted aryl, optionally substituted alkyl-Z— and optionally substituted aryl-Z—, where Z is N, O, S or Si, or R¹ and R² together form an optionally substituted hydrocarbon bridge or an optionally substituted α,ω-dioxahydrocarbon bridge between X and Y;

R³ and R⁴ are independently selected from the group consisting of hydrogen, halogen, optionally substituted alkyl, optionally substituted aryl, optionally substituted alkyl-Z′— and optionally substituted aryl-Z′—, where Z′ is N, O, S or Si, or R³ and R⁴ together form an optionally substituted hydrocarbon bridge or an optionally substituted α,ω-dioxahydrocarbon bridge between the two carbon atoms to which they are attached;

R⁵ and R⁶ are independently selected from the group consisting of hydrogen, optionally substituted alkyl and optionally substituted aryl, or R⁵ and R⁶ together with the nitrogen atom to which they are attached form an optionally substituted azacycloalkyl group; or an enantiomer or diastereomer thereof or a salt, optionally a pharmaceutically acceptable salt, of any of these, wherein if either X or Y or both is O, [symbol] is a single bond and if X═O, R² is absent and if Y═O, R¹ is absent.

3-Deazaneplanocin A may be excluded from the scope of this aspect. Any one or more, optionally all, of the following compounds may be excluded from the scope of this aspect: aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (1R,2S,3R)-3-(6-amino-9H-purin-9-yl)-1,2-cyclopentanediol hydrochloride, 2′,3′-O-isopropylidene-3-deazaneplanocin A, (1S,2R,5R)-5-(6-amino-9H-purin-9-yl)-3-methylcyclopent-3-ene-1,2-diol hydrochloride. 3-Deazaneplanocin A, aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (±)-(1R,2S,3R)-3-(6-amino-9H-purin-9-yl)-1,2-cyclopentanediol hydrochloride, 2′,3′-O-isopropylidene-3-deazaneplanocin A and (1S,2R,5R)-5-(6-amino-9H-purin-9-yl)-3-methylcyclopent-3-ene-1,2-diol hydrochloride may all be excluded from the scope of this aspect.

Alkyl groups described herein may be C1 to C12 alkyl groups, or C1 to C8, C1 to C6 or C1 to C4. They may be for example methyl, ethyl, propyl, isopropyl, butyl (m, s or t) etc. They may be linear, or they may (except for C1 and C2) be branched or cyclic alkyl. They may optionally contain one or more double or triple bonds (i.e. they may be alkenyl and/or alkynyl). They may optionally be substituted with one or more substituents. Each substituent on the alkyl groups may, independently, be R—B— (where R is hydrogen or an alkyl group as described above or an aryl group as described below, both being optionally substituted and B is O, S, N or Si) or halogen (e.g. F, Cl, Br or I). In the event that B is N or Si, the other (i.e. hitherto undefined) position(s) on B may (each independently) have an alkyl or aryl group as described herein. The alkyl groups may be arylalkyl groups. They may be arylcycloalkyl groups. They may represent alkoxyalkyl or aryloxyalkyl or alkylaminoalkyl (e.g. mono- or dialkylaminoalkyl) groups or arylaminoalkyl groups or alkanethioalkyl groups or arylthioalkyl groups or alkylsilylalkyl (e.g. trialkylsilylalkyl) groups or arylsilylalkyl groups (e.g. trialkyl-, aryldialkyl- or diarylalkyl-silylalkyl groups). They may represent oligoether groups (e.g. H(CH₂CH₂O)ₙCH₂CH₂—) or oligoaminogroups (e.g. H(CH₂CH₂NH)ₙCH₂CH₂—) where n=1 to about 6. The total number of atoms (other than hydrogen but including heteroatoms) in the main chain of the alkyl group may be 3 to 20, or 3 to 12 or 3 to 8.

Aryl groups described may be monocyclic aromatic groups or they may be bicyclic, tricyclic or oligocyclic. They may (except for monocyclic instances) be fused ring aromatic groups. They may be carbocyclic or they may be heterocyclic. They may for example be phenyl, naphthyl, anthracyl, pyridyl, furyl, pyrrolyl, thiofuryl, imidazolyl, indolyl, quinolinyl, naphthyridyl etc. They may optionally be substituted with one or more substituents. Each substituent on the aryl groups may, independently, be R—B—, where R and B are as described above (under “alkyl groups”). They may be for example alkylaryl groups or di-, tri-, tetra- or penta-alkylaryl groups, or may be alkoxyaryl groups or alkoxyalkoxyaryl groups. They may be haloaryl groups.

R¹ and R² may be hydrogen, halogen, optionally substituted alkyl, optionally substituted aryl, optionally substituted alkyl-Z— or optionally substituted aryl-Z—, where Z is N, O, S or Si. In the event that Z is N or Si, the other (i.e. hitherto undefined) position(s) on Z may (each independently) have hydrogen, an alkyl group or an aryl group as described above. R¹ and R² may together form an optionally substituted hydrocarbon bridge or an optionally substituted α,ω-dioxahydrocarbon bridge between X and Y. The substituents may be alkyl, aryl, R—B— or halogen, as described above. The hydrocarbon bridge may have formula —(CH₂)ₙ—, where n is an integer. n may be between 1 and 6, or 2 and 6, 3 and 6, 4 and 6 or 3 and 5, e.g. 1, 2, 3, 4, 5 or 6. In some instances the bridge may have substituents as described above. The substituents themselves may form a ring, whereby the substituent on N9 of the ring system is a fused tricyclic ring system. In many embodiments, R¹ is hydrogen, and in some embodiments both R¹ and R² are both hydrogen. In some embodiments R² is an alkyl group having an oxygen substituent (e.g. hydroxyl, alkoxy or aryloxy).

R³ and R⁴ may, independently, be hydrogen, halogen (e.g. chloro, bromo, iodo or fluoro), optionally substituted alkyl, optionally substituted aryl, optionally substituted alkyl-Z′— or optionally substituted aryl-Z′—, where Z′ is N, O, S or Si. In the event that Z′ is N or Si, the other (i.e. hitherto undefined) position(s) on Z′ may (each independently) have hydrogen, an alkyl group or an aryl group as described above. R³ and R⁴ may together form an optionally substituted hydrocarbon bridge or an optionally substituted α,ω-dioxahydrocarbon bridge between the two carbon atoms to which they are attached. Broadly the choice of options for R³ and R⁴ is the same as for R¹ and R² above. In some embodiments, R³ and R⁴ are both alkoxy, aryloxy or together form an α,ω-dioxahydrocarbon bridge. Suitable bridges include typical protecting groups for vicinal diols, for example methylene acetal, eththylidene acetal or isopropylidene acetal (acetonide: —OC(Me₂)O—).

R⁵ and R⁶ may be hydrogen, optionally substituted alkyl or optionally substituted aryl. R⁵ and R⁶ may, together with the nitrogen atom to which they are attached, form an optionally substituted azacycloalkyl group. The ring of the azacycloalkyl group may have about 3 to about ring members, or 4 to 8, 5 to 8 or 5 to 7 members. In many embodiments R⁵ and R⁶ are both hydrogen whereby N6 represents a primary amino group. In other embodiments N6 represents a secondary or tertiary amino group. Broadly the choice of options for R⁵ and R⁶ is the same as for R¹ and R² above with the exception that they may not be halogens or form a α,ω-dioxahydrocarbon bridge.

As described herein the term “Cancer” refers to malignant neoplasm, or a group of cells that display uncontrolled division and growth beyond the normal limits, ie: abnormal proliferation of cells invasion, intrusion on and destruction of adjacent tissues, and sometimes metastasis where the cancer cells have spread to other locations in the body via lymph system or blood. Most cancers form a tumor but some, like leukemia, do not. For the purpose of the invention cancer refers to cells where EZH2 has been upregulated and or FBXO32 has been downregulated. For example in cancer cells present in bone, lung, breast, gastric, colorectal, liver, prostate, cervical, brain, oral, esophagus, head and neck, lymphoma, leukemia, ovary, bladder, pancreatic, skin, sarcoma or any other cancers known to those skilled in the art.

Ectopic Expression of FBXO32

The present invention also provides a vector comprising a polynucleotide of the invention, for example an expression vector comprising a polynucleotide of the invention, operably linked to regulatory sequences capable of directing expression of said polynucleotide in a host cell. Vectors include plasmids, cosmids or any similar system known in the art.

Ectopic expression of FBXO32 may be achieved by constructing an expression vector as known in the art. Ectopic expression of the FBXO32 polynucleotide can be done by introducing a transgenic FBXO32 polynucleotide of SEQ ID NO. 1 of a functional variant thereof together with a promoter into a tumor cell. A functional variant is one that is able to decrease the expression of p21 polypeptide of SEQ ID NO. 5

While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation.” The cells into which have been introduced nucleic acids described below are meant to also include the progeny of such cells.

DNA Damaging Agents

DNA damaging agents are any agents that can cause damage to nucleic acids. Preferably DNA damaging agents are anticancer agents such as chemotherapy agents like, for example; Adriamycin (ADR), Etoposide (ETO, Nocodazole, cisplatin, platinum, carboplatin, gemcitabine, paclitaxel, docetaxel, vinorelbine, topotecan, or irinotecan; tyrosine kinase inhibitors (e.g., Axitinib, Bosutinib, Cediranib, Dasatinib, Erlotinib, Gefitinib, Imatinib, Lapatinib, Lastaurtinib, Nilotinib, semaxanib, sunitinib, vandetanib, vatalanib or any other suitable tyrosine kinase inhibitor); apoptosis inducing enzymes, for example TNF polypeptides, TRAIL (TRAIL R1, TRAIL R2) or FasL, Exisulind or other apoptosis inducing enzymes; micro-RNA that initiates apoptosis; or other chemotherapy agents such as those commonly known to a person skilled in the art. Alternatively they may be anticancer treatments such as radiotherapy, chest radiotherapy, surgical resection, the chemotherapy agents mentioned above or any combination of these.

Compositions of the Invention

compositions produced according to the invention can be administered for the treatment of cancer in the form of pharmaceutical compositions.

Thus, the present invention also relates to compositions including pharmaceutical compositions comprising a therapeutically effective amount of a compound that inhibits EZH2 to transactivate FBXO32 and or decreases p21 protein. Thereby a cell is sensitized to a DNA damaging agent. As used herein a compound will be therapeutically effective if it is able to affect cancer growth either in vitro or in vivo.

In one embodiment the EZH2 inhibitor in the compound is a MicroRNA. Preferably the MicroRNA is miR-101.

In another embodiment the EZH2 inhibitor in the compound is Isoliquiritigenin.

In another embodiment the EZH2 inhibitor in the compound is S-adenosylhomocysteine hydrolase inhibitor 3-Deazaneplanocin A (DZNep) as described above of formula I:

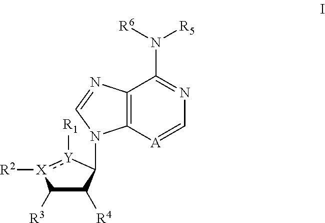

wherein:
X and Y are independently C or O,

### A is C or N;

[symbol] is a single bond or a double bond;
R¹ and R² are either absent or independently selected from the group consisting of hydrogen, halogen, optionally substituted alkyl, optionally substituted aryl, optionally substituted alkyl-Z— and optionally substituted aryl-Z—, where Z is N, O, S or Si, or R¹ and R² together form an optionally substituted hydrocarbon bridge or an optionally substituted α,ω-dioxahydrocarbon bridge between X and Y;
R³ and R⁴ are independently selected from the group consisting of hydrogen, halogen, optionally substituted alkyl, optionally substituted aryl, optionally substituted alkyl-Z′— and optionally substituted aryl-Z′—, where Z′ is N, O, S or Si, or R³ and R⁴ together form an optionally substituted hydrocarbon bridge or an optionally substituted α,ω-dioxahydrocarbon bridge between the two carbon atoms to which they are attached;
R⁵ and R⁶ are independently selected from the group consisting of hydrogen, optionally substituted alkyl and optionally substituted aryl, or R⁵ and R⁶ together with the nitrogen atom to which they are attached form an optionally substituted azacycloalkyl group;
or an enantiomer or diastereomer thereof or a salt of any of these,

wherein if either X or Y or both is O, [symbol] is a single bond and if X═O, R² is absent and if Y═O, R¹ is absent, and

wherein said compound is not 3-deazaneplanocin A or aristeromycin or 3-deazaaristeromycin hydrochloride or (1S,2R,5R)-5-(6-amino-9H-purin-9-yl)-3-(methoxymethyl)cyclopent-3-ene-1,2-diol hydrochloride or (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol or (1R,2S,3R)-3-(4-amino-1H-imidazo[4,5-c]pyridin-1-yl)cyclopentane-1,2-diol or (±)-(1R,2S,3R)-3-(6-amino-9H-purin-9-yl)-1,2-cyclopentanediol hydrochloride or 2′,3′-O-isopropylidene-3-deazaneplanocin A or (1S,2R,5R)-5-(6-amino-9H-purin-9-yl)-3-methylcyclopent-3-ene-1,2-diol hydrochloride.

In one embodiment the DNA damaging agent in the compound is a chemotherapeutic agent. Preferably the chemotherapeutic agent is selected from the group consisting of Adriamycin, Etoposide, Nocodazole, cisplatin, platinum, carboplatin, gemcitabine, paclitaxel, docetaxel, vinorelbine, topotecan, irinotecan, Axitinib, Bosutinib, Cediranib, Dasatinib, Erlotinib, Gefitinib, Imatinib, Lapatinib, Lastaurtinib, Nilotinib, semaxanib, sunitinib, vandetanib, vatalanib, TNF polypeptides, TRAIL (TRAIL R1, TRAIL R2) or FasL, Exisulind or apoptosis inducing micro-RNA.

In a preferred embodiment the DNA damaging agent in the compound is Adriamycin.

In a preferred embodiment the DNA damaging agent in the compound is Etoposide,

In a preferred embodiment the DNA damaging agent in the compound is Nocodazole,

Pharmaceutical forms of the invention suitable for injectable use include sterile aqueous solutions (where water soluble) or dispersions and sterile powders for the extemporaneous preparation of sterile injectable solutions and or one or more carrier. Alternatively, injectable solutions may be delivered encapsulated in liposomes to assist their transport across cell membranes. Alternatively or in addition such preparations may contain constituents of self-assembling pore structures to facilitate transport across the cellular membrane. It must be stable under the conditions of manufacture and storage and must be preserved against the contaminating/destructive action of microorganisms such as, for example, bacteria and fungi.

The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active peptides in the required amount in the appropriate solvent with several of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and freeze-drying, to yield a powder of the active ingredient plus any additional desired ingredient from previously sterile-filtered solution thereof.

When the active ingredients, in particular small molecules contemplated within the scope of the invention, are suitably protected they may be orally administered, for example, with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active molecules in such therapeutically useful compositions is such that a suitable dosage will be obtained. Preferred compositions or preparations according to the present invention are prepared so that a dosage unit form contains between about 0.1 μg and 20 g of active compound.

The tablets, troches, pills, capsules and the like may also contain binding agents, such as, for example, gum, acacia, corn starch or gelatin. They may also contain an excipient, such as, for example, dicalcium phosphate. They may also contain a disintegrating agent such as, for example, corn starch, potato starch, alginic acid and the like. They may also contain a lubricant such as, for example, magnesium stearate. They may also contain a sweetening agent such a sucrose, lactose or saccharin. They may also contain a flavouring agent such as, for example, peppermint, oil of wintergreen, or cherry flavouring.

When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparaben as preservatives, a dye and flavouring such as, for example, cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents may also include any and all solvents, dispersion media, coatings, antibacterials and/or antifungals, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated.

It is especially advantageous to formulate parenteral compositions in dosage unit form for ease of administration and uniformity of dosage. Dosage unit form as used herein refers to physically discrete units suited as unitary dosages for the mammalian subjects to be treated, each unit containing a predetermined quantity of active material calculated to produce the desired therapeutic effect in association with the required pharmaceutical carrier. The dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 pg to about 2000 mg/ml of carrier. In the case of compositions containing supplementary active ingredients, the dosages are determined by reference to the usual dose and manner of administration of the said ingredients.

The compounds and compositions may be adapted to be administered to the lungs directly through the airways by inhalation. Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluents such as lactose or starch. Inhalable dry powder compositions may be presented in capsules and cartridges of gelatin or a like material, or blisters of laminated aluminum foil for use in an inhaler or insufflators. Each capsule or cartridge may generally contain between 20 pg-10 mg of the active compound. Alternatively, the compound of the invention may be presented without excipients.

The inhalable compositions may be packaged for unit dose or multi-dose delivery. For example, the compositions can be packaged for multi-dose delivery in a manner analogous to that described in GB 2242134, U.S. Pat. No. 6,632,666, U.S. Pat. No. 5,860,419, U.S. Pat. No. 5,873,360 and U.S. Pat. No. 5,590,645 (all illustrating the “Diskus” device), or GB2178965, GB2129691, GB2169265, U.S. Pat. No. 4,778,054, U.S. Pat. No. 4,811,731 and U.S. Pat. No. 5,035,237 (which illustrate the “Diskhaler” device), or EP 69715 (“Turbuhaler” device), or GB 2064336 and U.S. Pat. No. 4,353,656 (“Rotahaler” device).

Spray compositions for topical delivery to the lung by inhalation may be formulated as aqueous solutions or suspensions or as aerosols delivered from pressurised packs, such as a metered dose inhaler (MDI), with the use of a suitable liquefied propellant. The medication in pressurized MDI is most commonly stored in solution in a pressurized canister that contains a propellant, although it may also be a suspension.

Aerosol compositions suitable for inhalation can be presented either as suspensions or as solutions and typically contain the active compound and a suitable propellant such as a fluorocarbon or hydrogen-containing chlorofluorocarbon or mixtures thereof, particularly hydrofluoroalkanes such as dichlorodifluoromethane, trichlorofluoromethane, dichlorotetrafluoroethane, and especially 1,1,1,2-tetrafluoroethane, 1,1,1,2,3,3,3-heptafluoro-n-propane and mixtures thereof.

The aerosol composition may optionally contain additional excipients typically associated with such compositions, for example surfactants such as oleic acid or lecithin and co-solvents such as ethanol. Pressurized formulations will generally be contained within a canister (for example an aluminum canister) closed with a metering valve and fitted into an actuator provided with a mouthpiece.

Peptides can also be delivered by protein delivery methods known in the art such as transfection, macromolecule delivery vehicles and other methods known to those skilled in the art.

The compositions may be for use in treating cancer. Use includes use of a composition of the invention for the preparation of a medicament or a pharmaceutically acceptable composition for the treatment of cancer. The preparation may further comprise a chemotherapeutic agent for the preparation of a medicament for the treatment of cancer.

Method for Treating a Patient with Cancer

On the basis of the above, the present invention provides a method for treating a patient with cancer, which comprises the step of: contacting the cells within and around a cancer with a composition as described above. Desirably, the EZH2 inhibitor is provided in a therapeutically effective amount.

An alternative form of the present invention resides in the use of the composition in the manufacture of a medicament for treating a patient with cancer preferably a medicament used in treatment to affect cells over expressing EZH2.

“Treatment” and “treat” and synonyms thereof refer to both therapeutic treatment and prophylactic or preventative measures, wherein the object is to prevent or slow down (lessen) a cancer condition. Those in need of such treatment include those already diagnosed with cancer or having cells over expressing MDM2.

As used herein a “therapeutically effective amount” of a compound will be an amount of active peptide that is capable of preventing or at least slowing down (lessening) a cancer condition, in particular increasing the average 5 year survival rate of cancer patients. Dosages and administration of an antagonist of the invention in a pharmaceutical composition may be determined by one of ordinary skill in the art of clinical pharmacology or pharmacokinetics. An effective amount of the inhibitor composition or compound to be employed therapeutically will depend, for example, upon the therapeutic objectives, the route of administration, and the condition of the mammal. Accordingly, it will be necessary for the therapist to titer the dosage and modify the route of administration as required to obtain the optimal therapeutic effect. A typical daily dosage might range from about 10 ng/kg to up to 100 mg/kg of the mammal's body weight or more per day, preferably about 1 μg/kg/day to 10 mg/kg/day.

### Detection Kits

Detection kits may contain antibodies, amplification systems, detection reagents (chromogen, fluorophore, etc), an enzyme capable of breaking down the natural extracellular matrix of the tissue to dissociate the cells (e.g., Trypsin, Elastase, Collagenase type 1 or 2, Protease, Pronase or any other suitable enzyme), dilution buffers, washing solutions, mounting solutions, counter stains or any combination thereof. Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, stratifying patient populations, diagnosis, prognosis, guiding therapeutic treatment decisions, and other applications.

Detection kits may further comprise magnetic beads such as dyna-beads or miltany beads or fluorophores for cell sorting techniques such as MACS or FACS and or secondary antibodies for extraction of cells with an existing antibody-antigen complex.

The detection kit may include a reagent such as an antibody capable of binding selectively a FBXO32 polypeptide which comprises a sequence capable of binding selectively a sequence set out in SEQ ID No 2, or the reagent may include a polynucleotide or a primer and a probe capable of binding selectively a FBXO32 polynucleotide. Preferably the polynucleotide is an mRNA allowing FBXO32 expression profiling of cells in vitro.

### Examples of Specific Embodiments

The present technology relates to a method of treating cancer by sensitizing human tumours to DNA damaging therapies through activating FBXO32 expression. Transactivation of FBXO32 through the inhibition of EZH2, a histone methyltransferase, decreases p21 protein induction which results in the sensitization of human tumours to chemotherapy. The method further provides a prognostic method to determine if the combination treatment would be effective.

Polycomb protein histone methyltranserase EZH2 is frequently overexpressed in human malignancy and its gene silencing activity is emerging as a crucial regulator of several signaling pathways important in tumorigenesis. However, it is largely unknown whether EZH2 has a role in cancer cell life and death decision in response to genotoxic stress. Here we show that EZH2-mediated gene repression plays a role in modulating DNA damage response. EZH2 depletion results in abrogation of cell cycle checkpoints, directing DNA damage response towards predominant apoptosis in both p53-proficient and p53-deficient cancer cells. Mechanistically, EZH2 regulates DNA damage response, at least in part, through transcriptional repression of FBXO32, which directs p21 for proteasome-mediated degradation. Furthermore, pharmacological depletion of EZH2 phenocopies the effects of EZH2 knockdown on cancer cell cycle checkpoints/apoptosis in a FBXO32-dependent manner. These data unravel a crucial role of EZH2 in determining the cancer cell outcome following DNA damage and suggest that inhibition of oncogenic EZH2 might severe as a potent strategy for improving conventional chemotherapy in a given malignancy.

EZH2 Depletion in Cancer Cells Directs DNA Damage Response Towards Apoptosis by Abrogating Cell Cycle Checkpoints

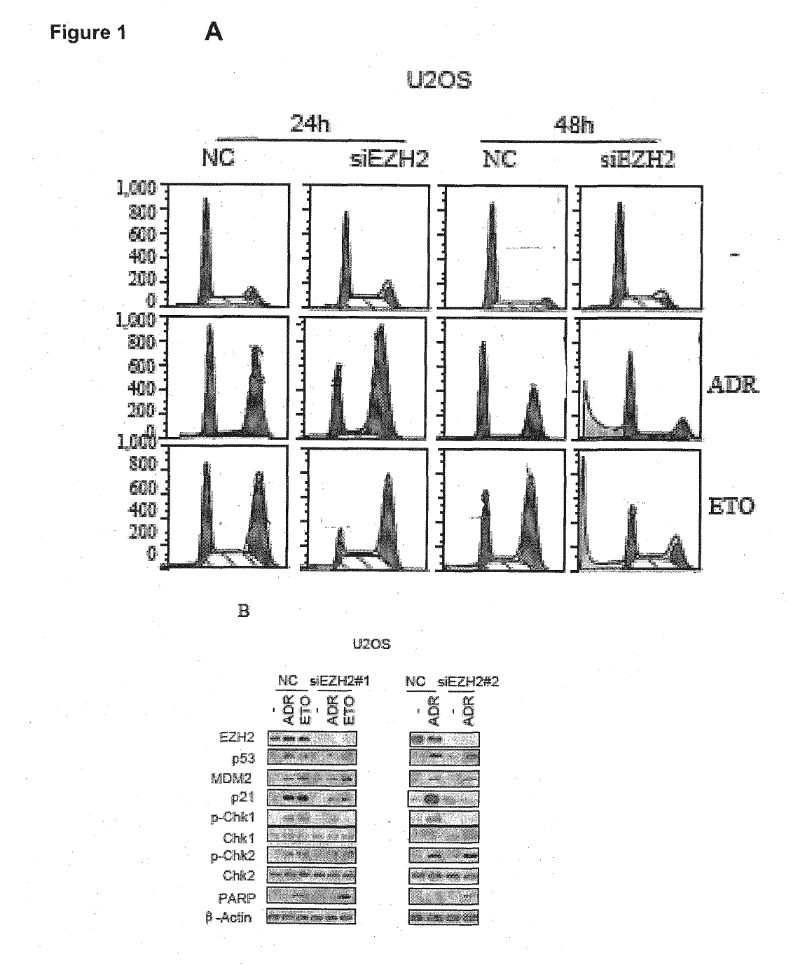

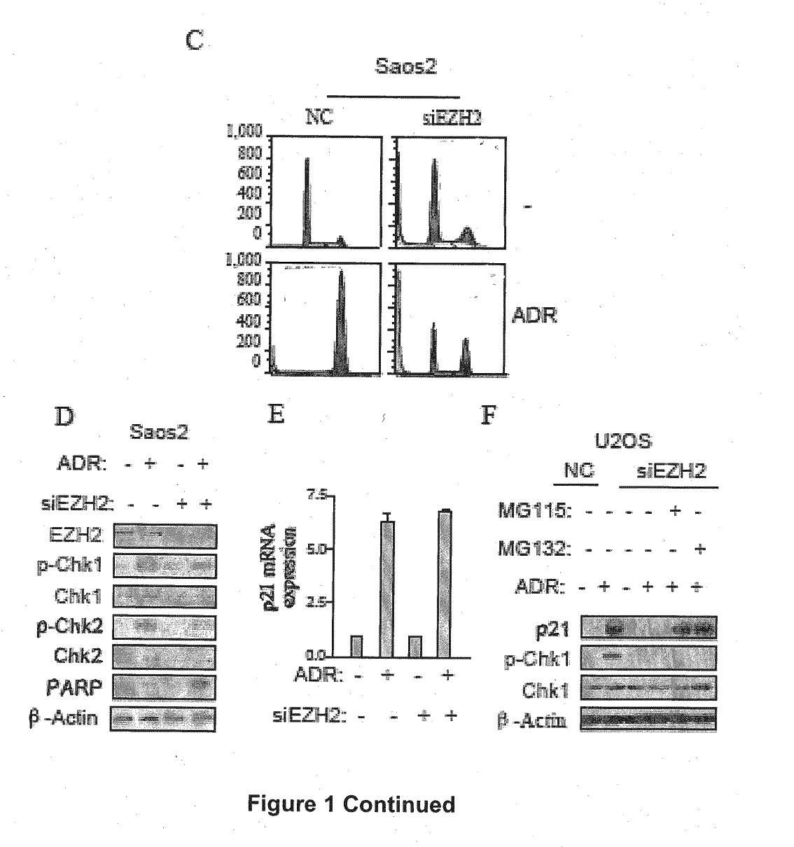

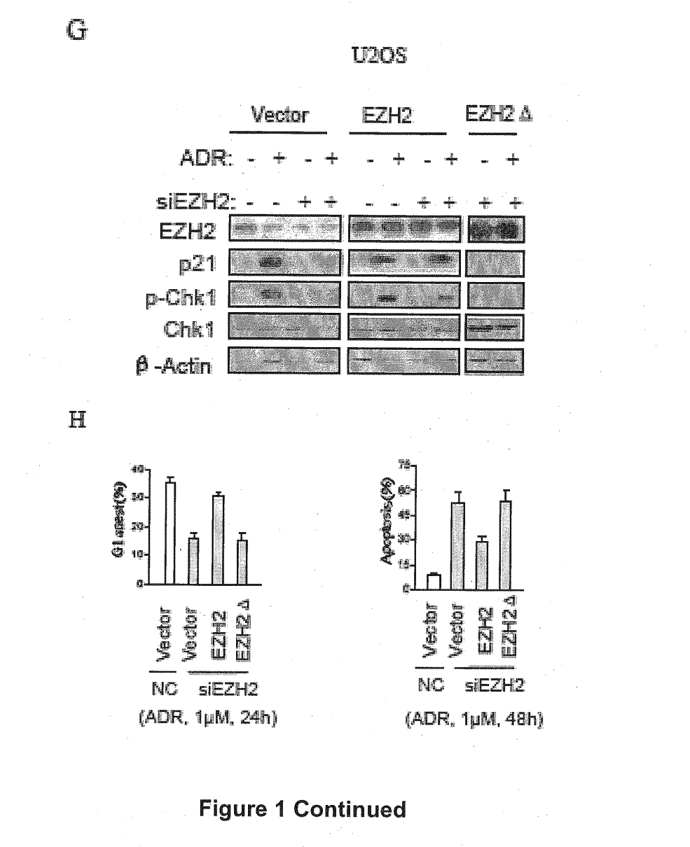

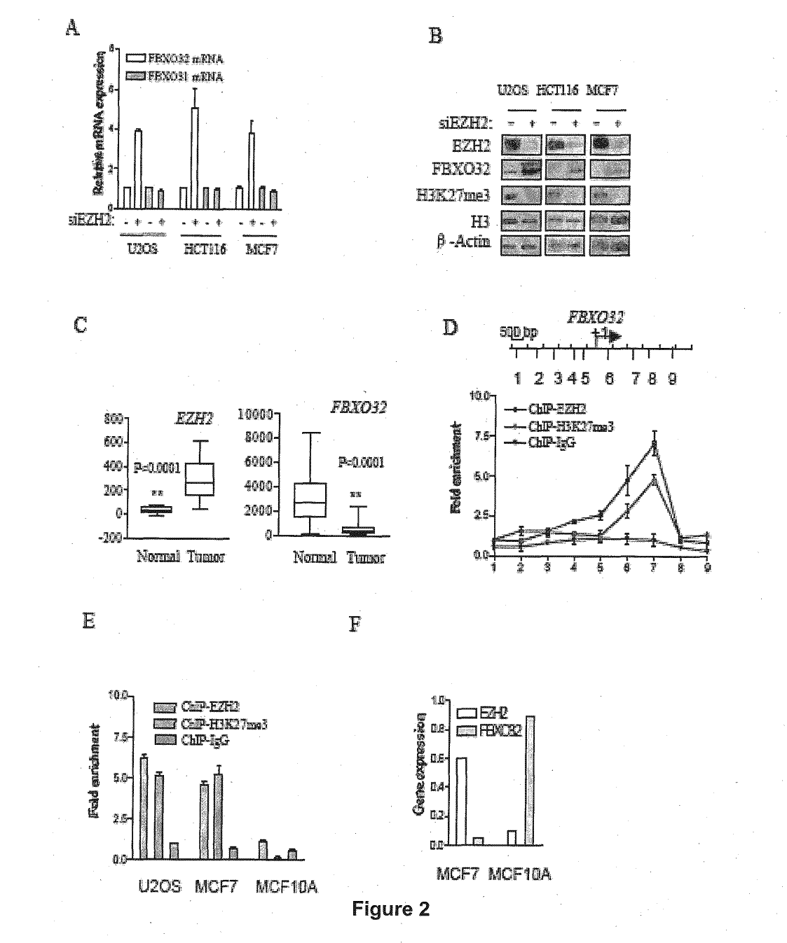

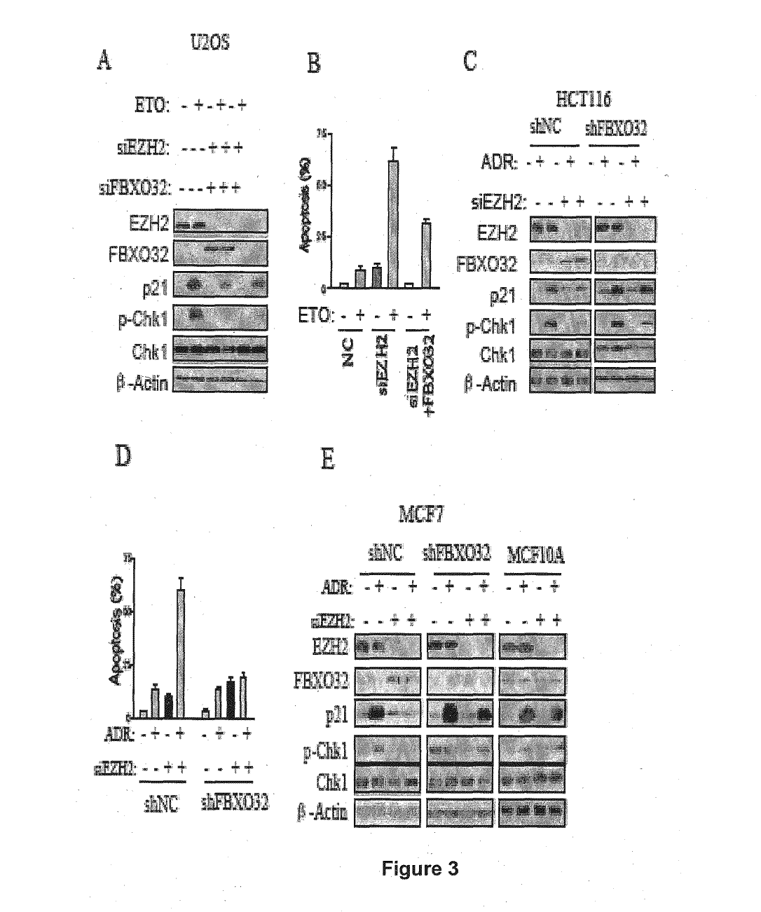

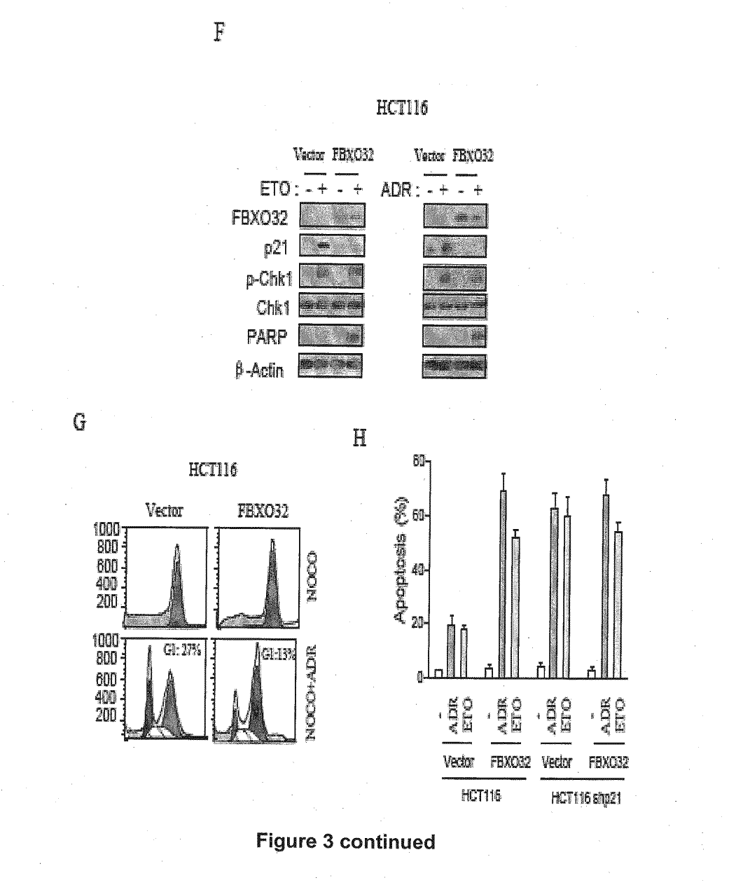

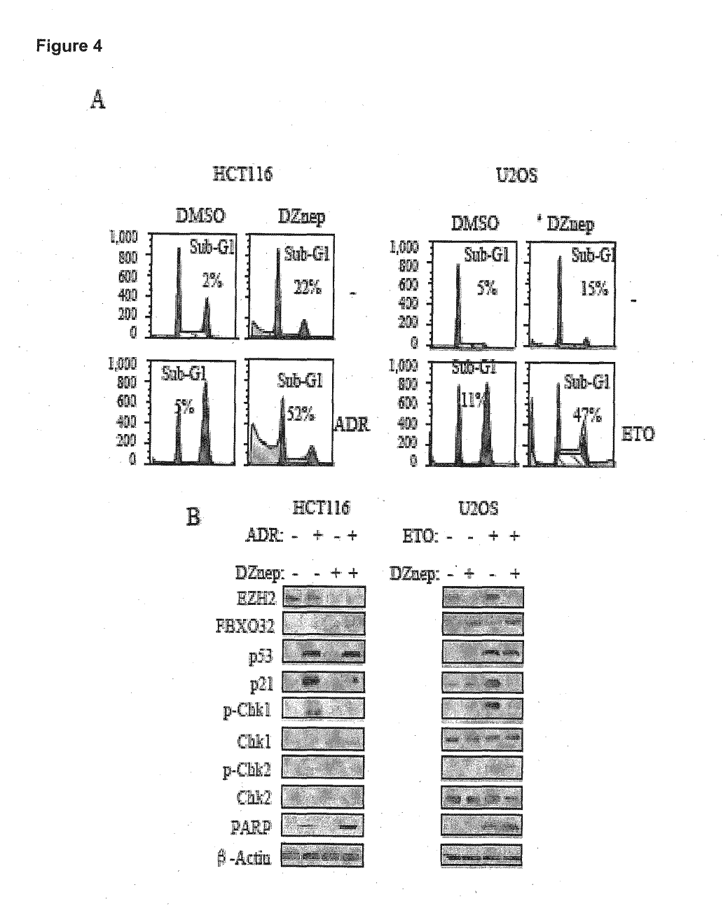

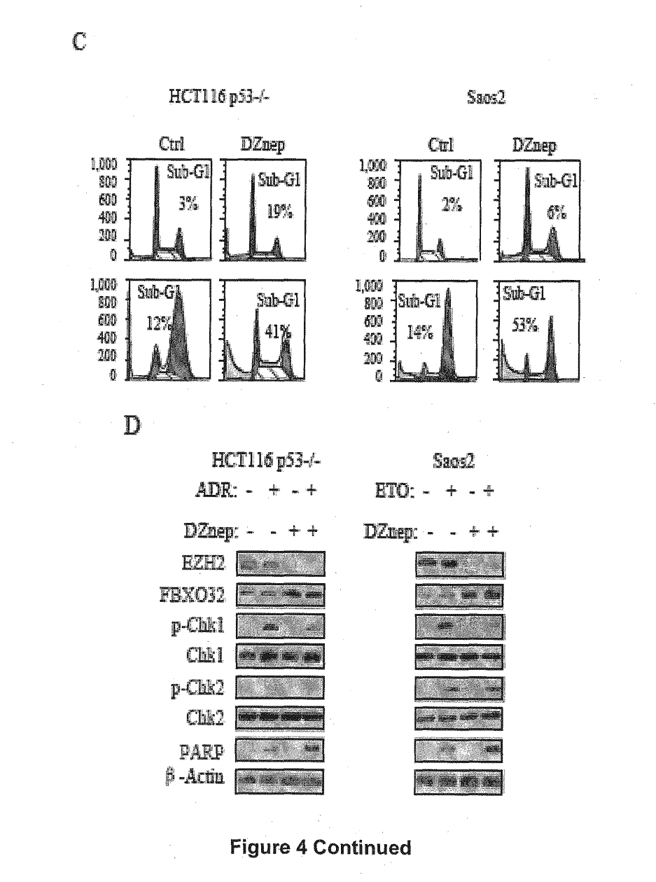

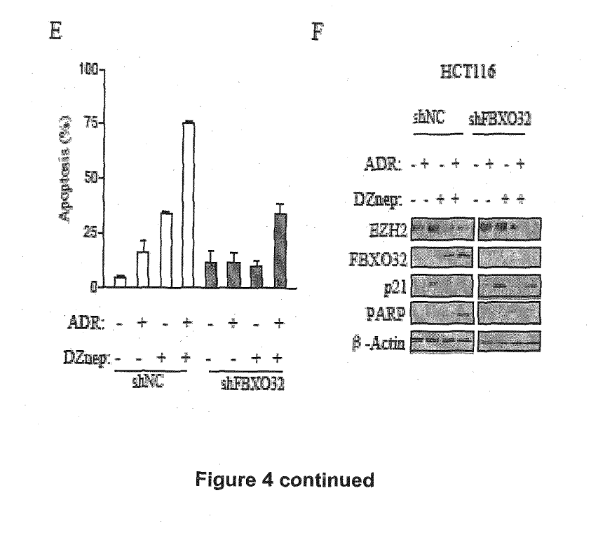

To assess a potential role of EZH2 in DNA damage response, we depleted EZH2 by RNA interference in p53 wild-type osteosarcoma U2OS cells and treated with DNA damaging agents Adriamycin (ADR) or Etoposide (ETO). While the control cells were mainly cell cycle arrested with little apoptosis, EZH2 knockdown cells exhibited a marked decrease in G1 arrest at 24 hour, followed by loss of G2/M arrest and robust apoptosis by 48 hour (FIG. 1A). Western blot analysis revealed that EZH2 depletion largely abolished ADR or ETO-induced p21 accumulation, though it had no effects on p53 and MDM2 induction (FIG. 1B, left panel). EZH2 depletion also blocked DNA damage-induced Chk1 phosphorylation, increased PARP cleavage, without affecting Chk2 phosphorylation (FIG. 1B). The results were further confirmed by using a different EZH2 siRNA (EZH2 siRNA UTR: 5-CGGTGGGACTCAGAAGGCA-3, or EZH2 siRNA#1 5-GACUCUGAAUGCAGUUGCU-3) to knockdown EZH2 activity (FIG. 1B, right panel). In addition, conversion of DNA damage-induced G2/M arrest to apoptosis upon EZH2 knockdown, with concurrent loss of Chk1 activation, was also seen in p53-deficient Saos-2 cells (FIGS. 1C and 1D). Thus, EZH2 knockdown abrogates DNA damage checkpoints and promotes apoptosis in both p53-proficient and p53-deficient cancer cells.

To further demonstrate the specificity of EZH2 knockdown, we performed rescue experiment by using another siRNA that targets the 5′-UTR region of EZH2 and thus does not affect ectopic EZH2. Indeed, the losses of both p21 and p-Chk1 induction by ADR in EZH2-depleted cells, as well as the corresponding changes in G1 arrest and apoptosis, were markedly restored by wild-type EZH2, but not by a deletion mutant lacking the catalytic SET-domain (EZH2ΔF) (FIGS. 1G and 1H). The findings support a role of EZH2 in favoring cell cycle arrest in response to DNA damage and indicate that this function of EZH2 requires the SET-domain which is essential for associated histone methylation (H3K27 trimethylation) and gene repression activity (Bracken et al., 2003). Of note, the downregulation of p21 protein was not due to decreased p21 mRNA level (FIG. 1E), and was restored upon treatment with proteasome inhibitors (FIG. 1F). This indicates that p21 regulation is post-translational and requires a proteasome-dependent mechanism for its downregulation. By contrast, reduced Chk1 activation does not seem to be caused by such a mechanism.

### EZH2 Depletion Results in Induction of FBXO32, a Target Directly Suppressed by EZH2 in Multiple Human Cancer Cells

We have identified F-Box protein FBXO32 as one of the gene targets repressed by EZH2 complex in breast cancer. F-box proteins are components of the SCF (SKP/Cullin/F-box protein) class of E3 ubiquitin ligases and have roles in substrate-recognization and degradation. Notably, its close family member FBXO31 has been recently shown to induce degradation of cycliD1 in DNA damage response (Santra et al., 2009). Therefore, we asked whether FBXO32 de-repression upon EZH2 depletion contributes to above phenotypes. We first wanted to confirm whether EZH2 depletion leads to FBXO32 induction in HCT116, U2OS and MCF7 cells. As shown in FIGS. 2A and 2B, levels of FBXO32 expression, but not FBXO31 were induced after EZH2 knockdown at both mRNA and protein levels in both cell lines, excluding a potential role of FBXO31 in p21 degradation in this context. Furthermore, we show that the repression of FBXO32 by EZH2 is clinically relevant as gene expression analysis of patient-derived normal colon and tumor samples revealed a reverse relationship between EZH2 and FBXO32, but not FBXO31 (FIGS. 2C and 2D). To demonstrate a direct repression of FBXO32 expression by EZH2, we performed chromatin immunoprecipitation (ChIP) assay and the results show that EZH2 and H3K27me3 were markedly enriched in the FBXO32 promoter in HCT116 (FIG. 2D), U2OS and MCF-7 cells, but not in non-cancerous MCF10A cells (FIG. 2E). This suggests that EZH2 represses FBXO32 expression selectively in cancer cells, which is consistent with a reverse correlation between EZH2 and FBXO32 expression levels in MCF-7 and MCF10A cells (FIG. 2F). Collectively, these findings identify FBXO32 as a bona fide direct target of EZH2 in multiple human cancers.

Induction of FBXO32 Following EZH2 Depletion is Required for p21 Degradation During DNA Damage

To validate a hypothetical role of FBXO32 in p21 protein regulation, we performed single or double knockdown of EZH2 and FBXO32 in U2OS, HCT116 and MCF-7 cells. In U2OS cells, diminished p21 level resulting from EZH2 knockdown during DNA damage was nearly completely rescued by concomitant knockdown of FBXO32 (FIG. 3A). Down-regulation of p-Chk1, however, was not rescued by concomitant FBXO32 knockdown (FIG. 3A), which was consistent with a partial rescue of apoptosis (FIG. 3B). A similar result was also found in HCT116 and MCF-7 cells in which FBXO32 was knockdown by shRNA targeting a different sequence (FIGS. 3C and 3D). By contrast, EZH2 knockdown in MCF10A cells had no effect on FBXO32 expression, and thus had no visible effect on ADR-induced p21 induction (FIG. 3E). Thus, the effect of EZH2 knockdown on FBXO32 induction and consequently reduced levels of p21 protein during DNA damage response is cancer specific.

Overexpression of FBXO32 Abolishes p21 Induction and Sensitizes DNA Damaging Agents—Induced Apoptosis

We next evaluated whether ectopic expression of FBXO32 would mimic the effects of EZH2 knockdown on p21, G1 arrest and apoptosis. Overexpression of FBXO32 in HCT116 cells resulted in inhibition of p21 induction by ETO and ADR, orchestrated by the increased PARP cleavage; but again Chk1 phosphorylation was not affected (FIG. 3F). FBXO32-overexpressing cells synchronized in mitosis with Nocodazole, which blocks exit from mitosis, showed reduced arrest in G1 phase after ADR treatment (FIG. 3G). FBXO32 overexpressing cells were more sensitive to ADR or ETO-induced apoptosis (FIG. 3H). HCT116 p21 shRNA cells were much more sensitive to ADR or ETO treatment, and FBXO32 overexpression did not further increase the magnitude of apoptosis (FIG. 3H). In summary, these data suggest that FBXO32-induced p21 downregulation is a key functional target of EZH2 knockdown, which plays a crucial role in causing cancer cell fate switch in response to DNA damage.

Pharmacologic Depletion of EZH2 by DZNep Phenocopies EZH2 Knockdown in Modulating DNA Damage Response

Thus far, we demonstrated an epigenetic mechanism of EZH2 in regulating cellular response to DNA damage induced by genotoxic agents. We next tested whether this finding can be translated into a viable treatment strategy to improve chemotherapeutic response. We have previously shown that histone methylation inhibitor Deazanaplanosin A (DZNep) can deplete EZH2 complex, resulting in reactivation of genes suppressed by EZH2 in cancer cells (Tan et al., 2007). We thus investigated whether DZNep treatment would phenocopy EZH2 knockdown and give rise to similar effects. As shown in FIG. 4, in both p53-proficient U2OS and HCT116 cells and p53-deficient Saos-2 and HCT116 cells, pretreatment with DZNep converted ADR or ETO-induced cell cycle arrest to marked apoptosis (FIGS. 4A and 4C). Similar to EZH2 knockdown, EZH2 depletion by DZNep was accompanied with FBXO32 induction, and largely abolished p21 and/or p-Chk1 induction by ADR or ETO in these cells (FIGS. 4B and 4D). By contrast, DZNep did not promote ADR or ETO-induced apoptosis in non-transformed MCF-10A, IMR90 and RWEP cells (data not shown). Again, p21 protein downregulation seen here was not due to a decrease in p21 mRNA, and was also prevented by MG132 (data not shown). Importantly, this effect of DZNep in HCT116 cells was largely abolished when FBXO32 was knockdown (FIGS. 4E and 4F), which is consistent with a crucial role of FBXO32 in DNA damage-induced apoptosis following EZH2 depletion. Thus, pharmacologic depletion of EZH2 by DZNep phenocopied the effects of genetic knockdown of EZH2 on FBXO32, p21, cell cycle checkpoints and apoptosis in DNA damage response. Although DZNep may not be a specific EZH2 inhibitor, it provides a proof of principle that development of specific small molecule inhibitor of EZH2 may provide benefits for sensitizing chemotherapy-induced apoptosis selectively in cancer cells.

Our data provides the first demonstration that EZH2-mediated gene silencing is critical for regulating the outcome of cellular DNA damage. A striking finding is that inhibition of EZH2 abrogates both p21 and Chk1 activation and promotes DNA damaging agents-induced apoptosis in both p53 wild type and p53-deficient cancer cells. Thus, these findings bear important implications for cancer treatment and indicate that therapeutic inhibition of EZH2 may be an attractive approach to sensitize cells to standard chemotherapy DNA damaging treatments in cancers that overexpress EZH2. These findings point out a potential application of EZH2 inhibitors that are under active development by pharmaceutical companies.

Mechanistically, this effect of EZH2, at least in part, is through the suppression of FBXO32-directed p21 degradation to maintain the cell cycle arrest rather than apoptosis in response to DNA damage. Thus, identification of EZH2-mediated FBXO32 repression in DNA damage-induced cell cycle checkpoint control through regulation of p21 stability suggests an epigenetic mechanism regulating DNA damage response that can be targeted to augment chemotherapeutic response. Importantly, because this regulation is cancer specific, targeting EZH2 is expected to result in selective sensitization in cancer cells, with a minimum effect on normal cells. Further investigation of the molecular mechanisms by which EZH2 regulates Chk1 activation is likely to provide additional insights into epigenetic regulation of DNA damage response.

Given the toxic side effect of chemotherapy, any sensitizer that can direct even a mild DNA damage response towards an apoptotic program would have the potential to enhance the efficacy of DNA damaging chemotherapeutic agents allowing less DNA damaging chemotherapeutic agents to be used and reduce the toxic effects. Furthermore, given a potential role of EZH2 in cancer stem cells, and that DNA damage-activated p21 is required for self-renewal of leukaemia stem cells, EZH2 inhibition might also have effect in overcoming chemoresistance phenotype typically seen in cancer stem cells that are believed to confer tumor recurrence after chemotherapy (Bao et al., 2006; Eramo et al., 2006).

Materials and Methods

Cell Culture and Drugs

The human colorectal cancer HCT116 cells were kindly provided by Dr. Bert Vogelstein (John Hopkins University, MD). Other cell lines used in this study, including human osteosarcoma U2OS and Saos-2 and breast cancer MCF7 were obtained from the American Type Culture Collection (ATCC) and maintained in Dulbecco's modified Eagle medium (DMEM) supplemented with 10% fetal bovine serum (FBS) and antibiotics in a 37° C. humidified incubator containing 5% CO₂. Human breast epithelial MCF10A cells were obtained from ATCC and maintained as recommended (Kadota et al 2010). Adramycin (ADR), Etopside (ETO), Nocodazole (Noco), proteasomal inhibitors MG132 and MG115 were purchased from Sigma-Aldrich.

Plasmids and Stable Cell Lines

pcDNA4/FBXO32-Myc was generated by RT-PCR using total RNA from normal colon tissue. Retroviral-mediated gene transfer was performed using pMN-GFP/IRES retrovirus vector-expressing FBXO32. Infected cells were sorted by GFP signals and expanded for in vitro studies. EZH2 wild-type and SET domain deletion mutant (EZH2Δ) plasmids have been described previously respectively (Sheaff et al 2000, Wu et al 2009). All constructs were confirmed by sequencing.

RNA Interference

Specific siRNA oligos targeting EZH2 (as described above) and FBXO32 (FBXO32 siRNA: 5_-GTCACATCCTTTCCTGGAA-3_) mRNAs were used. The non-targeting control was purchased from Dharmacon (Lafayett, Colo.). For EZH2 knockdown, a sequential twice knockdown is performed to secure efficient gene silencing. Cells were transfected with 100 nM final concentration of siRNA duplexes using Lipofectamine RNAiMax (Invitrogen) following the manufacturer's instructions. To generate FBXO32 shRNA stable cell lines, siRNA oligo targeting FBXO32 (sequence: CAGAAGATTATATGGCGCGAA) were cloned into the pSIREN-RetroQ retroviral expression vector (BD Bioscence) according to the manufacturer's instruction. Virally infected cells were selected in a medium containing 2 μg/ml puromycin individual drug-resistant clones were collected and expanded.

Immunoblot Analysis and Co-Immunoprecipitation

Protein extracts were prepared by lysis in RIPA buffer containing (50 mM Tris-HCl, pH 7.4, 150 mM NaCl, 1% Nonidet P-40, 0.5% sodium deoxycholate, 0.1% SDS, 1 mM EDTA, 50 mM NaF, 0.1 mM Na3VO4) and protease inhibitor cocktail (Roch). Lysates were resolved by SDS-PAGE, transferred onto a immobilon membrane (Millipore) and probed with indicated antibodies. For co-immunoprecipitation experiments, cells were lysed and incubated with the indicated antidodies and protein G-seperose beads overnight at 4° C. Beads were washed four times with lysis buffer. The bound proteins were dissolved in SDS sample buffer, resolved by SDS-PAGE, and immunoblotted with the indicated antibodies. Where indicated, proteosome inhibitors MG-132 (5 μM) or MG-115 (20 μM) were used to treat cells for 8 h before protein extraction. Antibodies used in this study include: anti-EZH2 (Cell Signaling), anti-p21 (Santa Cruz), anti-FBXO32 (Santa Cruz), anti-p53 (Santa Cruz), anti-MDM2 (Santa Cruz), anti-phospho-Chk1 (Cell signaling), anti-Chk1 (Santa Cruz), anti-phospho-Chk2 (Cell signaling), anti-Chk2 (Upstate), anti-H3 (Cell signaling), anti-PARP (Cell Signaling), anti-H3K27me3 (Upstate), anti-actin mouse monoclonal (Sigma).

Taqman Assay

Total RNA was isolated using Trizol (Invitrogen) and purified with the RNeasy Mini Kit (Qiagen). Reverse transcription was performed using an RNA Amplification kit (Ambion). Quantitative real-time PCR was assessed using the PRISM 7900 Sequence Detection System (Applied Biosystems) with specific probes from Applied Biosystem. Samples were normalized to the levels of GAPDH mRNA.

Flow Cytometry and Cell Cycle Analysis

Cells were harvested and fixed in 70% ethanol. Fixed cells were stained with propidium iodide (50 μg/mL) after treatment with RNase (100 μg/mL). The stained cells were analyzed for DNA content by fluorescence-activated cell sorting (FACS) in a FACS Calibur (Becton Dickinson Instrument, San Jose, Calif.). Cell cycle fractions were quantified using the CellQuest software (Becton Dickinson). For synchronization experiment, HCT116 cells expressing FBXO32 or empty vector were treated with Nocodazole (400 ng/ml) for 16 h, mitotically arrested cells were treated with ADR and then subject to FACS for cell cycle analysis.

Chromatin Immunoprecipitation (ChIP) Assay

ChIP assays were performed as described previously (Jiang et al 2008). Briefly, sonicated extracts were pre-cleared and incubated with antibodies specific to either EZH2 (Active motif, Carlsbad, Calif.), H3K27me3 (Upstate) or IgG control (sc-2027, Santa Cruz) at 4° C. overnight on a 360° C. rotator. The immunoprecipitated DNA was quantitated by real-time quantitative PCR using Applied Biosystems 7900HT Fast Real-Time PCR System (Applied Biosystems). The enrichment of EZH2 or H3K27me3 binding at the examined regions was quantitated relative to the input amount.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

While the invention has been described with reference to specific methods and embodiments, it will be appreciated that various modifications and changes may be made without departing from the invention.

### REFERENCES

Abbas T. & Dutta A. (2009). p21 in cancer: intricate networks and multiple activities. Nat Rev Cancer, 9, 400-14.
Bao S., Wu Q., McLendon R. E., Hao Y., Shi Q., Hjelmeland A. B., Dewhirst M. W., Bigner D. D. & Rich J. N. (2006). Glioma stem cells promote radioresistance by preferential activation of the DNA damage response. Nature, 444, 756-60.
Beuvink I., Boulay A., Fumagalli S., Zilbermann F., Ruetz S., O'Reilly T., Natt F., Hall J., Lane H. A. & Thomas G. (2005). The mTOR inhibitor RAD001 sensitizes tumor cells to DNA-damaged induced apoptosis through inhibition of p21 translation. Cell, 120, 747-59.
Bracken A. P. & Helin K. (2009). Polycomb group proteins: navigators of lineage pathways led astray in cancer. Nat Rev Cancer, 9, 773-84.
Bracken A. P., Kleine-Kohlbrecher D., Dietrich N., Pasini D., Gargiulo G., Beekman C., Theilgaard-Monch K., Minucci S., Porse B. T., Marine J. C., Hansen K. H. & Helin K. (2007). The Polycomb group proteins bind throughout the INK4A-ARF locus and are disassociated in senescent cells. Genes Dev, 21, 525-30.
Bracken A. P., Pasini D., Capra M., Prosperini E., Colli E. & Helin K. (2003). EZH2 is downstream of the pRB-E2F pathway, essential for proliferation and amplified in cancer. Embo J, 22, 5323-35.
Bunz F., Fauth C., Speicher M. R., Dutriaux A., Sedivy J. M., Kinzler K. W., Vogelstein B. & Lengauer C. (2002). Targeted inactivation of p53 in human cells does not result in aneuploidy. Cancer Res, 62, 1129-33.
Bunz F., Hwang P. M., Torrance C., Waldman T., Zhang Y., Dillehay L., Williams J., Lengauer C., Kinzler K. W. & Vogelstein B. (1999). Disruption of p53 in human cancer cells alters the responses to therapeutic agents. J Clin Invest, 104, 263-9.
Cao Q., Yu J., Dhanasekaran S. M., Kim J. H., Mani R. S., Tomlins S. A., Mehra R., Laxman B., Cao X., Yu J., Kleer C. G., Varambally S. & Chinnaiyan A. M. (2008). Repression of E-cadherin by the polycomb group protein EZH2 in cancer. Oncogene, 27, 7274-84.
Cao R. & Zhang Y. (2004). The functions of E(Z)/EZH2-mediated methylation of lysine 27 in histone H3. Curr Opin Genet Dev, 14, 155-64.
Chen H., Tu S. W. & Hsieh J. T. (2005). Down-regulation of human DAB2IP gene expression mediated by polycomb Ezh2 complex and histone deacetylase in prostate cancer. J Biol Chem, 280, 22437-44.
Eramo A., Ricci-Vitiani L., Zeuner A., Pallini R., Lotti F., Sette G., Pilozzi E., Larocca L. M., Peschle C. & De Maria R. (2006). Chemotherapy resistance of glioblastoma stem cells. Cell Death Differ, 13, 1238-41.
Fan S., Chang J. K., Smith M. L., Duba D., Formace A. J., Jr. & O'Connor P. M. (1997). Cells lacking CIP1/WAF1 genes exhibit preferential sensitivity to cisplatin and nitrogen mustard. Oncogene, 14, 2127-36.
Fujii S., Ito K., Ito Y. & Ochiai A. (2008). Enhancer of zeste homologue 2 (EZH2) down-regulates RUNX3 by increasing histone H3 methylation. J Biol Chem, 283, 17324-32.
Graves P. R., Yu L., Schwarz J. K., Gales J., Sausville E. A., O'Connor P. M. & Piwnica-Worms H. (2000). The Chk1 protein kinase and the Cdc25C regulatory pathways are targets of the anticancer agent UCN-01. J Biol Chem, 275, 5600-5.
Hermand D. (2006). F-box proteins: more than baits for the SCF? Cell Div, 1, 30.
Ho M. S., Tsai P. I. & Chien C. T. (2006). F-box proteins: the key to protein degradation. J Biomed Sci, 13, 181-91.
Jariel-Encontre I., Bossis G. & Piechaczyk M. (2008). Ubiquitin-independent degradation of proteins by the proteasome. Biochim Biophys Acta, 1786, 153-77.
Jascur T., Brickner H., Salles-Passador I., Barbier V., E1 Khissiin A., Smith B., Fotedar R. & Fotedar A. (2005). Regulation of p21(WAF1/CIP1) stability by WISp39, a Hsp90 binding TPR protein. Mol Cell, 17, 237-49.
Kawabe T. (2004). G2 checkpoint abrogators as anticancer drugs. Mol Cancer Ther, 3, 513-9.
Kleer C. G., Cao Q., Varambally S., Shen R., Ota I., Tomlins S. A., Ghosh D., Sewalt R. G., Otte A. P., Hayes D. F., Sabel M. S., Livant D., Weiss S. J., Rubin M. A. & Chinnaiyan A. M. (2003). EZH2 is a marker of aggressive breast cancer and promotes neoplastic transformation of breast epithelial cells. Proc Natl Acad Sci USA, 100, 11606-11.
Li J., Tan J., Zhuang L., Banerjee B., Yang X., Chau J. F., Lee P. L., Hande M. P., Li B. & Yu Q. (2007). Ribosomal protein S27-like, a p53-inducible modulator of cell fate in response to genotoxic stress. Cancer Res, 67, 11317-26.
Min J., Zaslaysky A., Fedele G., McLaughlin S. K., Reczek E. E., De Raedt T., Guney I., Strochlic D. E., Macconaill L. E., Beroukhim R., Bronson R. T., Ryeom S., Hahn W. C., Loda M. & Cichowski K. (2010). An oncogene-tumor suppressor cascade drives metastatic prostate cancer by coordinately activating Ras and nuclear factor-kappaB. Nat Med, 16, 286-94.
Mukhopadhyay U. K., Senderowicz A. M. & Ferbeyre G. (2005). RNA silencing of checkpoint regulators sensitizes p53-defective prostate cancer cells to chemotherapy while sparing normal cells. Cancer Res, 65, 2872-81.
Pasini D., Cloos P. A., Walfridsson J., Olsson L., Bukowski J. P., Johansen J. V., Bak M., Tommerup N., Rappsilber J. & Helin K. (2010). JARID2 regulates binding of the Polycomb repressive complex 2 to target genes in ES cells. Nature, 464, 306-10.
Rodriguez R. & Meuth M. (2006). Chk1 and p21 cooperate to prevent apoptosis during DNA replication fork stress. Mol Biol Cell, 17, 402-12.
Sancar A., Lindsey-Boltz L. A., Unsal-Kacmaz K. & Linn S. (2004). Molecular mechanisms of mammalian DNA repair and the DNA damage checkpoints. Annu Rev Biochem, 73, 39-85.
Santra M. K., Wajapeyee N. & Green M. R. (2009). F-box protein FBXO31 mediates cyclin D1 degradation to induce G1 arrest after DNA damage. Nature, 459, 722-5.
Seoane J., Le H. V. & Massague J. (2002). Myc suppression of the p21(Cip1) Cdk inhibitor influences the outcome of the p53 response to DNA damage. Nature, 419, 729-34.
Simon J. A. & Lange C. A. (2008). Roles of the EZH2 histone methyltransferase in cancer epigenetics. Mutat Res, 647, 21-9.
Suva M. L., Riggi N., Janiszewska M., Radovanovic I., Provero P., Stehle J. C., Baumer K., Le Bitoux M. A., Marino D., Cironi L., Marquez V. E., Clement V. & Stamenkovic I. (2009). EZH2 is essential for glioblastoma cancer stem cell maintenance. Cancer Res, 69, 9211-8.
Tan J., Yang X., Zhuang L., Jiang X., Chen W., Lee P. L., Karuturi R. K., Tan P. B., Liu E. T. & Yu Q. (2007). Pharmacologic disruption of Polycomb-repressive complex 2-mediated gene repression selectively induces apoptosis in cancer cells. Genes Dev, 21, 1050-63.
Tse A. N. & Schwartz G. K. (2004). Potentiation of cytotoxicity of topoisomerase i poison by concurrent and sequential treatment with the checkpoint inhibitor UCN-01 involves disparate mechanisms resulting in either p53-independent clonogenic suppression or p53-dependent mitotic catastrophe. Cancer Res, 64, 6635-44.
Varambally S., Dhanasekaran S. M., Zhou M., Barrette T. R., Kumar-Sinha C., Sanda M. G., Ghosh D., Pienta K. J., Sewalt R. G., Otte A. P., Rubin M. A. & Chinnaiyan A. M. (2002). The polycomb group protein EZH2 is involved in progression of prostate cancer. Nature, 419, 624-9.
Viale A., De Franco F., Orleth A., Cambiaghi V., Giuliani V., Bossi D., Ronchini C., Ronzoni S., Muradore I., Monestiroli S., Gobbi A., Alcalay M., Minucci S. & Pelicci P. G. (2009). Cell-cycle restriction limits DNA damage and maintains self-renewal of leukaemia stem cells. Nature, 457, 51-6.
Waldman T., Lengauer C., Kinzler K. W. & Vogelstein B. (1996). Uncoupling of S phase and mitosis induced by anticancer agents in cells lacking p21. Nature, 381, 713-6.
Weiss R. H. (2003). p21Waf1/Cip1 as a therapeutic target in breast and other cancers. Cancer Cell, 4, 425-9.
Wouters B. G., Giaccia A. J., Denko N. C. & Brown J. M. (1997). Loss of p21Waf1/Cip1 sensitizes tumors to radiation by an apoptosis-independent mechanism. Cancer Res, 57, 4703-6.
Wu Z. L., Zheng S. S., Li Z. M., Qiao Y. Y., Aau M. Y. & Yu Q. (2009). Polycomb protein EZH2 regulates E2F1-dependent apoptosis through epigenetically modulating Bim expression. Cell Death Differ.
Yang X., Karuturi R. K., Sun F., Aau M., Yu K., Shao R., Miller L. D., Tan P. B. & Yu Q. (2009). CDKN1C (p57) is a direct target of EZH2 and suppressed by multiple epigenetic mechanisms in breast cancer cells. PLoS ONE, 4, e5011.
Yu J., Cao Q., Mehra R., Laxman B., Yu J., Tomlins S. A., Creighton C. J., Dhanasekaran S. M., Shen R., Chen G., Morris D. S., Marquez. V. E., Shah R. B., Ghosh D., Varambally S. & Chinnaiyan A. M. (2007). Integrative genomics analysis reveals silencing of beta-adrenergic signaling by polycomb in prostate cancer. Cancer Cell, 12, 419-31.
Zhou B. B. & Elledge S. J. (2000). The DNA damage response: putting checkpoints in perspective. Nature, 408, 433-9.

1. A method of inducing apoptosis of a cell comprising the steps of:
increasing expression of an FBXO32 polypeptide and decreasing expression of a p21 polypetide.

2. The method of claim 1 whereby the expression of FBXO32 is increased by applying an inhibitor of a Polycomb protein histone methyltranserase (EZH2) expression of SEQ ID NO. 3 to the cell resulting in the decrease of p21 expression.

3. The method of claim 2 further comprising the step of adding a DNA damaging agent to the cell.

4. The method of claim 2 or 3 wherein the EZH2 inhibitor is a MicroRNA.

5. The method of claim 4 wherein the MicroRNA is miR-101 of SEQ ID NO. 8.

6. The method of claim 2 or 3 wherein the EZH2 inhibitor is Isoliquiritigenin.

7. The method of claim 2 or 3 wherein the EZH2 inhibitor is S-adenosyl homocysteine hydrolase inhibitor 3-Deazaneplanocin A (DZNep) of formula I: 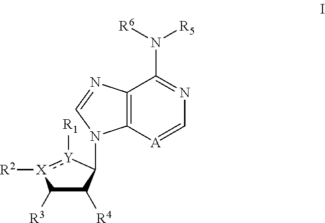
wherein:
X and Y are independently C or O,
A is C or N; [symbol]
is a single bond or a double bond;
R¹ and R² are either absent or independently selected from the group consisting of hydrogen, halogen, optionally substituted alkyl, optionally substituted aryl, optionally substituted alkyl-Z— and optionally substituted aryl-Z—, where Z is N, O, S or Si, or R¹ and R² together form an optionally substituted hydrocarbon bridge or an optionally substituted α,ω-dioxahydrocarbon bridge between X and Y;
R³ and R⁴ are independently selected from the group consisting of hydrogen, halogen, optionally substituted alkyl, optionally substituted aryl, optionally substituted alkyl-Z′— and optionally substituted aryl-Z′—, where Z′ is N, O, S or Si, or R³ and R⁴ together form an optionally substituted hydrocarbon bridge or an optionally substituted α,ω-dioxahydrocarbon bridge between the two carbon atoms to which they are attached;
R⁵ and R⁶ are independently selected from the group consisting of hydrogen, optionally substituted alkyl and optionally substituted aryl, or R⁵ and R⁶ together with the nitrogen atom to which they are attached form an optionally substituted azacycloalkyl group;
or an enantiomer or diastereomer thereof or a salt of any of these,
wherein if either X or Y or both is O, [symbol] is a single bond and if X═O, R² is absent and if Y═O, R¹ is absent, and
wherein said compound is not 3-deazaneplanocin A or aristeromycin or 3-deazaaristeromycin hydrochloride or (1S,2R,5R)-5-(6-amino-9H-purin-9-yl)-3-(methoxymethyl)cyclopent-3-ene-1,2-diol hydrochloride or (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol or (1R,2S,3R)-3-(4-amino-1H-imidazo[4,5-c]pyridin-1-yl)cyclopentane-1,2-diol or (±)-(1R,2S,3R)-3-(6-amino-9H-purin-9-yl)-1,2-cyclopentanediol hydrochloride or 2′,3′-O-isopropylidene-3-deazaneplanocin A or (1S,2R,5R)-5-(6-amino-9H-purin-9-yl)-3-methylcyclopent-3-ene-1,2-diol hydrochloride.

8. The method of anyone of claims 3 to 7 wherein the DNA damaging agent is a chemotherapeutic agent.

9. The method of claim 8 wherein the chemotherapeutic agent is selected from the group consisting of Adriamycin, Etoposide, Nocodazole, cisplatin, platinum, carboplatin, gemcitabine, paclitaxel, docetaxel, vinorelbine, topotecan, irinotecan, Axitinib, Bosutinib, Cediranib, Dasatinib, Erlotinib, Gefitinib, Imatinib, Lapatinib, Lastaurtinib, Nilotinib, semaxanib, sunitinib, vandetanib, vatalanib, TNF polypeptides, TRAIL (TRAIL R1, TRAIL R2) or FasL, Exisulind or apoptosis inducing micro-RNA.

10. The method of claim 1 whereby the expression of FBXO32 is increased by ectopic expression of FBXO32 of SEQ ID No. 1.

11. A compound comprising a composition capable of increasing expression of FBXO32 polypeptide and decreasing expression of p21 polypeptide and a DNA damaging agent.

12. The compound of claim 11 whereby the composition capable of increasing the expression of FBXO32 polypeptide is an EZH2 inhibitor.

13. The compound of claim 12 wherein the EZH2 inhibitor is a MicroRNA.

14. The compound of claim 13 wherein the MicroRNA is miR-101 of SEQ ID NO. 8.

15. The compound of claim 12 wherein the EZH2 inhibitor is Isoliquiritigenin.

16. The compound of claim 12 wherein the EZH2 inhibitor is S-adenosylhomocysteine hydrolase inhibitor 3-Deazaneplanocin A (DZNep) of formula I: 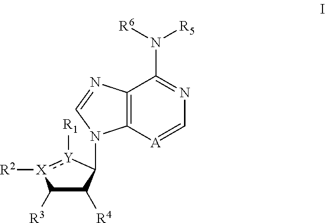
wherein:
X and Y are independently C or O,
A is C or N; [symbol]
is a single bond or a double bond;
R¹ and R² are either absent or independently selected from the group consisting of hydrogen, halogen, optionally substituted alkyl, optionally substituted aryl, optionally substituted alkyl-Z— and optionally substituted aryl-Z—, where Z is N, O, S or Si, or R¹ and R² together form an optionally substituted hydrocarbon bridge or an optionally substituted α,ω-dioxahydrocarbon bridge between X and Y;
R³ and R⁴ are independently selected from the group consisting of hydrogen, halogen, optionally substituted alkyl, optionally substituted aryl, optionally substituted alkyl-Z′— and optionally substituted aryl-Z′—, where Z′ is N, O, S or Si, or R³ and R⁴ together form an optionally substituted hydrocarbon bridge or an optionally substituted α,ω-dioxahydrocarbon bridge between the two carbon atoms to which they are attached;
R⁵ and R⁶ are independently selected from the group consisting of hydrogen, optionally substituted alkyl and optionally substituted aryl, or R⁵ and R⁶ together with the nitrogen atom to which they are attached form an optionally substituted azacycloalkyl group;
or an enantiomer or diastereomer thereof or a salt of any of these,
wherein if either X or Y or both is O, [symbol] is a single bond and if X═O, R² is absent and if Y═O, R¹ is absent, and
wherein said compound is not 3-deazaneplanocin A or aristeromycin or 3-deazaaristeromycin hydrochloride or (1S,2R,5R)-5-(6-amino-9H-purin-9-yl)-3-(methoxymethyl)cyclopent-3-ene-1,2-diol hydrochloride or (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol or (1R,2S,3R)-3-(4-amino-1H-imidazo[4,5-c]pyridin-1-yl)cyclopentane-1,2-diol or (±)-(1R,2S,3R)-3-(6-amino-9H-purin-9-yl)-1,2-cyclopentanediol hydrochloride or 2′,3′-O-isopropylidene-3-deazaneplanocin A or (1S,2R,5R)-5-(6-amino-9H-purin-9-yl)-3-methylcyclopent-3-ene-1,2-diol hydrochloride.

17. The compound of anyone of claims 11 to 16 wherein the DNA damaging agent is a chemotherapeutic agent.

18. The compound of claim 17 wherein the chemotherapeutic agent is selected from the group consisting of Adriamycin, Etoposide, Nocodazole, cisplatin, platinum, carboplatin, gemcitabine, paclitaxel, docetaxel, vinorelbine, topotecan, irinotecan, Axitinib, Bosutinib, Cediranib, Dasatinib, Erlotinib, Gefitinib, Imatinib, Lapatinib, Lastaurtinib, Nilotinib, semaxanib, sunitinib, vandetanib, vatalanib, TNF polypeptides, TRAIL (TRAIL R1, TRAIL R2) or FasL, Exisulind or apoptosis inducing micro-RNA.

19. The compound of anyone of claims 11 to 16 wherein the DNA damaging agent is Adriamycin.

20. The compound of anyone of claims 11 to 16 wherein the DNA damaging agent is Etoposide.

21. The compound of anyone of claims 11 to 16 wherein the DNA damaging agent is Nocodazole.

22. The compound of claim 11 whereby the composition capable of increasing the expression of FBXO32 polypeptide is an expression vector expressing an FBXO32 polynucleotide of SEQ ID No. 1.

23. A method of predicting the effectiveness of compound of the invention comprising the steps of:
a. determining a first expression profile of FBXO32 polypeptide in a subject who is not diagnosed with cancer;
b. determining a second expression profile of FBXO32 polypeptide in a subject who is diagnosed with cancer; and
c. comparing the first and second expression profile whereby when the second expression profile is less than the first expression profile the subject who is diagnosed with cancer will benefit from treatment with the compound of anyone of claims 11 to 22.

24. A kit to determine an expression profile of FBXO32 in vitro, comprising a reagent capable of binding selectively an FBXO32 polynucleotide of SEQ ID NO. 1.

25. The kit of claim 24 further comprising a detection reagent capable of binding selectively a p21 polynucleotide of SEQ ID NO. 5.


SEQUENCE LISTING (12 sequences)

SEQ ID NO: 1 - DNA, 38089 bp, Homo sapiens
gcactcccggagcctgcaacgcttgagatcctctccgcgcccgccaccccgcagggtgccccgcgccgttcccgccgccccgccgcccccgtcgcgggcccctgcaccccgagcatccgccccgggtggcacgtccccgagcccaccaggccggccccgtctccccatccgtctagtccgctcgcggtgccatgccattcctcgggcaggactggcggtcccccgggcagaactgggtgaagacggccgacggctggaagcgcttcctggatgagaagagcggcagtttcgtgagcgacctcagcaggtgaggggacccgctaccaacttccagcccccgcgaaactgacccgggctggtctggggggcgcagggacggcgaatgaggggcaggcagggctggagcgggctgaggagggagaccctgacccggagggtgggactacggggcccgatttctgcagaggtcttcggaccaccggagacagggaattcggtttctcttattgttcaaggtcagtgtctccaagtggacacgactccttacagaacacctggtccctctgagagtctgatgacattcgcggatcgtgtccccaaacgttcgctcaaacttaatcacacagctttagtggtccctgctttaaggacaccggagtcgcgcactttgcccatgtgggggcgcgggcgggtgggctgagccccttcaccgcggggtggcccggctcccagcgcccaagcgctgtggacgcgacaggtgacgggatccgacgaaggtcccgcctggagcgcttccaggccctgcccaggcacgggcaaccacgaagtggccgggaggatgagtaaccaggaagggacggccagatggtccctggggcagccgcttactgggcggacttgcgagcggtggaggcttggggctgcagaggtgcaaaggagggaagggggcaggggatgaatcccacaagagctctgcttctgggaggacagggctcttgcacctgtttcctcggtggccctctcggtcggtctgctttgtatagagtgcactccgtttccaggggtgcctgtcagctgcgaaaagcctgcctggccccggtgagcctctggattcgaggcctcacctgagctctgtggtctggcgggaaacctgaggtccgctgcgctccctgctcagcttggctaacgatgccagcgccggctagtacgcaaggtagcctggaatttctcagcccctgacctcgacagttgactttcagagactctcagcatttagcccggtgagcgggggcgatgaaagtgattccagaatggactggtgcagccagagtcacccttgggagccttttcctgccacctttggttaaaaccatgggcccctgcacccttaaattgacttcaatggagaattctgtttctgggccaggttcccgcttactgagatttaccagtcgttgcttggatgaagcattggcaataaattttgagaaaactatcaagaaaatgtggttattttggcaacttgaactcccgtgcctcccatttgtttgtgatttgtaatgcatcaaattcccattgtctcacaatgtaaagaaagacattatttaattctgggggtctgtccggagagacttgagcgtattaaccaccagcatataaccatttacgttggagttacttggaggctgtcccaggggagtttagggaccaccataaggaaatcttatttacgtaaaatcaagtaatttatttacgggaaagtaagaccaactgctattgacagactatgtcttttatcgactggagttaccaattatgcctaaattattctccctcaacaaaatgggaaccttccttccttcctccttgttcctaaggacaatgatctaatatgttggtactttatttctgtatacaagttcaggttctttctagaaaagtgccgaaagggtctgaagagtcatgttagagctgctcacccaaatataattgcaggatgctcctcagtggagaacagatgattgagttcagcagaagatttaaaatgtagggtcgtattaatacttaccctactcgggaggctgaggtgggaggatggcttgtgctcagaagttcaaggctgcagcgagtcatgactgtgccactgcgctccagcctgggccacagagcgagaccctgtctctaaaaataaataaaaatttaaaatttgttttaaaataccctataccggcatcaggagagaaactggtgtcactctcctttagaaatagtggtatctctgtcttcacagtatggaagacctgaagatggtcttaacttaccacaaaagtttagactgggctggaatcaggtggtttcctgcacagcccccctcccgtgtcctgttcatgagcctctttttcttcccatttcacacctgagctggagctgtcacatccatcatcattaccgcaaatagtcattgatatactgaccacctgtggtcctttgagtcagcgtttaggaatcatttggtagcctcctagcaaagcagctggctgtttgtttgcagggtatttttttttccagcaactgtaaacaatcccttccctgcttagcatgagaagcctgcaacttcttaaaagcagttggccttctgtggtctgggtttgtcatctcaaatatcctcactgtttttggtcttggtgatgtttccccgtgatgtcaggtttggactgcgtgcctgcctgcacaggtgaagctgaatgcctctcctgttatgagaaaacaaaaggcaggcccttaagcggggagggaccgtgaaacagagaactacagagacatggtagactgacttctctaattccacagaccattctgcaaacggcccaaacaagagcccgggatgaaggatgcatttggtggttttcagcctggggttattgtttcgtgttgaccaagtcttgtccacacagcacacctattctggactcagctggccatcttctctgtggtctaggactccaaccttctccagtgtagaatgttacaaatccagtcccagccatgtgtccaggagtgagatcatgggaggatttaagataagcttaaagcaggaccccttctcaaaaatgttgaaattatataaaatagaaggggaatggcaagtgttaaatatgctggggcagatttaaaatgcaaggaaagagcttggcgtgaattgggatgatccaagaatgcttcaaggaagtggtggttcaggcacccgcacagccttcctcagcaaccaccccctcaccgcccacccccaggatgagtaacctaagctgtctgagcctctgccactcttttctaagcctacacttctgtcattgaagactgactcaaatgttactttactgtatggctgaggctggacccggaagcaggaagtttaccttggtgactggataaaaataatagttaagattttatgagtgcttactgtgtgcaaggcactgttctatgagcgttatacactaggctgtttgtttcaccagagcattccgggtgctagccttgtgagtgtggcgatctagaaatgcgtcagtaaattggtgtagaaatctctactggctttaatcaggctatataactacaattaaacctctgggggcacgtcacagctttcttaaatggtttttattgcaaatgaaatgagaaatagggctttattttttttttcattcccttctttatctgctgccaaaacagacttatttatggtctgattttgaattttttctacatttttaaaaagttgccttaaagtatcttggtatgtggtaatgtggggctgtgcagtggatctttttcttattctaggcctgtgcctataaaaatcaagattagtaaatagtgactacaaaatagcgacttgctggctattattgaatttagtgaagaaagcagcctcgttgagatggttttgcttattaaaaaaaaaaaaatcctgtgctccaattcctaaatggtggtggtttttcttgttaaaaatttcagttgtaattttccattggctttgcattttcttgaaaaggacacgattgaactctcaaaatagcctctaactgaatatattattgcttaaaagaaaacttataagtaggcaacacaaataaaccccatagcccttctttccaggcccttcctccggaatctaaaacctaataccaagtgaaaatatagggtttctctttggttaaaacttttctacattctacggtgagagaacatcatcaattatgtaaaattctttctaaactagcttttgaaatgagtcactcaggcttaaagcaggttattgtagcttctgaggaaagtgagtgctccaattcctttaaagaacgttttcaattggaaaagagtaagatgaaatctaaatatttgaacttgctaagtaatgatcacaatctatggcccaggctcagaaaatctgctactgaaccttaaatgttgagaataagacaacctcattttatgattctctacatgtggaatttatactgtaggtttttgttgtttgtgggggttgtgtgtgttctttctaagtaagccttcatgtggtatttttgcacacaaaatgttctagctttgctttctaaagctgttctcagctgtggtacatcatggaccactcaaatgaccttccatcgtttgctgtttttctcaacgttgattgaaatgtcttaagttagcctcaggaattcagttctgttctttgttctgtccctcttttccttctgaccttggagcagataatcagctggtcagtgattctgtacccctatagtcatgcaaatagacagaaacacactcatgtgttgtacaaagcctggcccaaaatactcatccttggcttgcttttgaaggtgcctctgagaggacatttctgcaggactctatctgaaacctaaaaatatcctgtttccctgaatgaaaaggagaaagctattgagtttagtaggaaactcaataaatggtattaagcacggccttatgaaggaagattaacaaatgattatttatcatgtttgtccttaagggtccagaaaagcaacactgagtatattgttggagatgtcacatgtaagcatattagcaagatacttatactaaccgaagtgctacttgagagactattgtattaataacaaaaagatactagaagttgcttcaaatcttttctcttctaccatttacacaatgccaaggaaatagagaagtgcagatgtacataattgtacccaacagatgtatatctgaaaaagatgtaaatcaagttacattttaaattcagcagagaaactcagaacattaaaggagtataaaatatcggtggtatagcaaaaaaaaaaaaacccctaatttatgttttaccagttggcattataaatcctgttttacatactttgtgggggttgagggagttagggaacctatcttatcatacctcagtatttctcctgcattccttctgtggaaattattcagagatttactttgaagttacattgtatagtagaatagttcattacccctcaaatgattgctcagcccactgaggcaaataagcgtctggcccgagatgagcctgcccatccttcagtgacctgtgaattaggcaggtacatccaatgtctgttcagcccttataaatttatcaattataagtttaccttgtatagtcaagacccaaggtcttcacctcacagcatgatgcctagtgtgttttagtcaaggcactacataaaacctaagcaactggcactacacccaaagcatctcctagaaatggagtttataagtcagatttttgtaaacgtgattgtgttttaaagacatgttagaaggatgctacttaaaagcagcttaagtatactttaaatggatgaattgtatggtatgtgaagtttatctcaatgaaactttttttttttctaaagaagcagcagcagcttggggtagatacttttgcaaaatgaaggcattgtgtagtacagcgataccagtaatcacccctccccccattcgtcatttttgtttaaagtaatatggtaatactctgcattgctcttagttgaagagtacagtgctgtgtgtcaaattacaatgcaaggcactgttagggaattcaaaagaagtagaagctcccatccctgccttccccacagatgtgaacatgtttcatcctaaaggagaagcctgtgactgtatagactcttttttctttgagtgatcattttattgttttattttgtttgagaagcctgtatgtttgtttgtcttataactcagtgagttatttatagaaacagctcaggttcatgtatagctgttacagctcatcctctttgtcttctgttattcatagttactgcaacaaggaggtatacaataaggagaatcttttcaacagcctgaactatgatgttgcagccaagaagagaaagaaggacatgctgaatagcaaaaccaaaactcagtgtaagccatttgttttgaacttcagaaaaaagctgtttgaagatagttatgattggttcttggatgaaaaaaaataacacctcccatgtcacacatctgcctcagtgttttactgtgtgtcactgacagaggttactgagatctcagcactcagaaacctggctgtgcattttacaagcaagattaacatgttctttatagtcttgtttgtaagtcagttgctagttgagggcctttttttcctgctagtcattcgtatttgacataggttattttacagtgatgtggtcagctgaatcagttccagtatgtacttttattttgaactgttttgaggctttgaaaacagatttttctccctctttccttgtataaatcttattaacatttctagatactatatataagtatcagcagagaatataaagtggtattaatagcaggaattacaggttattttatctgtaatgcatgttttcctcatgttatctgtttgattttcatggacttatgtaagtttcaggtttggcatttaaaataaacttgtacttgagtattaggggtgttttttttttttttggtgttttttgtttgtttgtttttgttttttgagacagagtcttgctctctcacccaggctggagtcagtggcatgatcttagctcactgcaacctccgcctcttgggtttaagtgattctcgtgcctcagcctcctgagtagctgggattacaagcacctgccaccatacccggctaatttttgtgtttttagtagagacagggtttcgccatgttgcccaggctggtctcaaactccagggctcaagcgatctaccccgctctgttcacagcatgtaactcagtctgtcaaagcctaatttcatttttgcggggggacggagtctcgctctgtcgcccaggctggagtgcagtggcacaatcttggctcactgcaacctctgcctcccgggttcaagtgattctcctacctcagcctcccaagtagctgggattacaggtgcctgccaccacgcctggctaatttttgaatttttagaagagacggggtttcaccatgttggccaggctggtctcgaactcctgacctctagtgatccgcctgcctcaacctcccacagtgctgggatttcaggcatgagccactgcatctggcctgtaatttcttatggcataatgctttagaaaaaaaaatatagtagtttggagctttggtatatgaaatctggctctcaaagatttagagtgcttcagacaaagcaacttatagccccagtatttctacactgcagaaattaagcacttgaaacttcttcagttgtcagtagactccttatcttttttgtcaaaacttttaaaactttggaataaatgaaatgaaatgtcttaaatcttgagcatgttgcagatgttaaagctgtgtattctttttgtctctgtagatttccaccaagaaaaatggatctatgttcacaaaggaagtactaaagaggtgagcatcccattcatttactaagttatactgaaccttatcccttcctgatagcgcagggagggatatggtagggagggaaattacttcggatcatgagagtcttcttggatgtgggagatggcagctattagtctattatttagccaaccatcagagtttagaacagagagagaaagaagcaaaagtcactaggtggaccctctagtcgttcaatgagattatgtatgtaaaagtgctttataaagattgacatgcccatctcaaccatattattatattgccctgggttttgcctcagggcaaggtctcccttttgttctaagatctctttggacttcaactcagttaaatttcagtcctggaagcgctaatcactaaatatagtagaagttttcagtgcctgctcagtgtgatgtgggaggggacaagacattgtaataactacatttgtatggtactttataaagcccttttatgtatattttctctctgcacaggcatttgctaaaataactaggatacaggcaatatgaaagaatttctatgaccgtcagggacctcagtgaacatctagtcccctctgtgatttgaggtccagaggggggtcactgatattcccaagaccacccagatactcagagcagagctgggactttatactcccaattccctctgtgttcataggttaaaagatcaaaacttttgcttcctcccaaaaccttataaacacttctgatgatcacagtgagaaaagcactattggtataaggcaaggtggtgacacagttggtgcaggtaggtctaattctcctcccaggagtacgttacagtcaccaaccatcatggcctcccatgtcccgtaccctgctcccagtctttccttaataagcaattacttatgatttttccagactaatatgtggaaaaattataagcaaggtcccaaatggtacaagtcacgaaaagggggtgtttgacatgtgaggggctgactgaggcagctgctggctgtgttacccatcttggtggctctcaagggcttgtgggtacaggaaagaagccagtgccccaggtcacctaagacaggcatcaagctcattcggcaaaatccagttgggttgttctaactggctcattcttacttgggtgacctctgcatggcaggagtaacttctgtgcctttgttttcatgttcagcgccatggatattgcaccctgggggaagctttcaacagactggacttctcaactgccattctggattccagaagatttaactacgtggtccgggtaagtctcaagtgcctaaaggctcatccaggctctttcccaagttgaatcatctctttgggttgtcttgaagtagtttccccaaaaaattactttttaggaccctaagaattttgaggcaagcagaaaacagacccctcaatctccttcttccccaacgtacttcaagttcttaagtgagtcctactaggatgtccctgagattggccaggatgggcaggggccgccataataccccagtgagtcggttaaagagccattctcttcaagtgaaatttcacaccaggccccttccgagcccaccctgtggatacgtgggaacatgtcacaggcaacgctggccctggcaacagccttagcaagagagaattcagcaggaagcaagccctaaagcactttggactgtttcaaaaccctgtatctctgactgttctcactgtctccctcttgtcctacttcagctgactctgggggacccccaaggcattctcagatgaggccacaaaactggatgaattaaagcacctccgtgttttgtgcctgtggttcccttccacccccatgtgataacagtattcagtcccatgtccacctaaagcttaaaactgcctgttttggccgggtgcggtggctcacacctgtaatcccagcactttgggaggccaaagcgggcggatcacctgaggtcaggagtttgagaccagcctggacaacatgatgaaatcctgtctctactaaaaatacaagaaaattagccaggcgtggtggcgggtgcctgtaatcccagctacttgggaggctgaggcaggagaatcgcttgaacccgggaggcagacattgcagtgagctgagattgcgccattgcactccagcctgggcaacaggagtgagactccatgtaaaaaaaaaaaaaaaaaaaaaaaaatctgactggatctgttgctttccattttaaaccgtattttgagaaaaaaaaaaaaaggctgggtgcattggctcacgcctgtaatcccagcactttgggaggctaagacaagcagatcatgaggtcaggagttcgagcccagcctggccaacatagtgaaaccccgtctctactaaaaatacaaaaaaaaattatccaggtgtggtggtgggcgcctgtaatcccagctactcgggaggctgaggcaggagaatcgcttgaatccaggaggcggaggttgcagtgagcgagatggcgccattgcactccagcctgggcgacagtgcgagactctgtctcaaaaaagaaaaagttaatgagcaaaatattaatgatgaaggtagtaaagcaaatcagcttaaccatgactgtcacattattatttctaattggtttattattttgttctccctgcctgccctccaaagctcccttgtgtatttttgtcagcagtcaacctgggaggtagcagctgtttgggttttaggcttagaagtaggagggaattttccccctgtgggtgtggttggtttggtttttattttctttttcctgggttgttgtgcttctcggtgccatgtcactgagtcagatgattgcagaggctcagccagggtgatgacattcaaggccccttgaggcctggatccttcacctgtcatctgggcagatgcaggcttggctgtccaaagtgacctgataaaaacgcaggcctgcaagaagcaggttaggagctgtgagctgagcctgtgaccacatgagcagatctattcactcttcggctaagctcttcttccaatccaaaagggataaaaataagagaccttcttgggtgaaatgttcatgccattcagtgtgactctgcaagttcacggaggtcacctagtcctcctagggcccacagactagtctaggctggttggaaatgggcccaggatgatctggaaacttccattgctaagaatccaaatagtttccatcttctaggtattagaggaagacacttagatctgatgtcttattacaaactgatcttcaaaatggtcagtttttagatggtcttcctttctacctgcttttccttttacaagaaataaagcaaggaacaagcatcatgatagtgaaaatctatttcatcaatatgttcattttcaccttttatcttttcaataaccctttaaaatatgggtgcttttattatctctctttaaacaaatggcagtacagtaagataaaatgacttgtccatgatcacagagggatgaaatcaagacttgaaatctgggcagtcagagtctacacttttaactgctaccatgagttatctgtgagcagaggagctgaagtaaaatagcctgagggcaggtcctagctccactgatttcaaactgtctcaacctccctaagacatggtttcctcctctgtaagaatgacttacctcaacttcccggggtagctggggaaactcagtgatataaacagaagtactttggacactagaatctatctgctgtgcaaagtcagccgctaatgtttactgttacccacttaccaaaattattgatttccccctaaaacgcttttcagttgagttgattaaaatgttttctacttttaaaaaactaattggtgaatctggcaattgcttaagactcagcacagagaacacgtatctaagagtatctaattgtaaaatacatcatgagcgctttgctttaaataatcttagagaaactgctgaatgctttcaaagttgttaggttcagaaatagtggaaaaatatttctattttgcaaagaacaatttttccccatcactattagagacctctgtaatttgaggaataatatgttatttttatgaagaacttaaaacattttaatgatagccactgtgttttgaggttttccttcttaggaactcagtatacttctcatacattcttatccttaaaccaactctgcaaagtagcttgccagtatattatccccatcgtactactttgacctaatgttgtttaaatgctacaatgattactggttttgctaaggagctaaactgattttaagggccatttgggaaaaaaacaaaaacaaaaaaaacattcaaagccttgagagccaaatacattttctgaaaatctgttatgttcatgctggttgtcatcttccaaaaaagagacatgcagaggtaataaaagagatggctaacactgctatctcaaaatattatcatttttatcactcagcatgaaaatttcacgaatgtttagagtcagaaaattttttactataacttagaaagatgtttatataacatttctacatcattttcactggctacaagctacatgcattggccctgctccctcccctacacagccccaatgacataaagacagttctagctatgtgtttgaaagatttcacatctctagaaacagcaacttacctacttgtggaaggacatttaaaacatagtttaccctatacctcaagaccaacacattttggaaattcccttgaggaactggaaggattccagaggccaagccagtcagactggaagtttgtaaccatgagtaggtgagtgctgactaaccaggccacagagaggtcagaggatcgagtgagtcttgggcatcaagtcaaccaggagaaggtttcggagaccatcacccatgtaatggctctgcagtgagcctgctgtgaccccagtggttctcagtcctggctgcacattcaaatcaccttcgaagcttgttagactacagaagcccacctctggagcttccgatttaattagttgaaggtggtacccaggcattgctacttttaaaatatctgcctggtgatcccaatcagttttcaagtttacagaccagctcagaaactgctttggagcccagatattatgttttgcatattaatgagaatttttaaacaaagtgcactgagacattaaactctagatacgacttctggggcgtgtactgtggtccatcaggaatttagtgatggaaagaggacagcccctcctatattccagctcaaagttcgaagcttctttttttgtagtactttctgcaaatcaatcattttacttctctaaactgtaaatctaggataaccatacctgtattgaagcatttttatgagaatgaaaaaataaaatgtaaaaataaaggcagtgggtgtgaaagtgttatataaatttcaaaatgcaatgtaacttacttttcagaaagttaagtaggattttagcatatccaaccatctttccagggtgattaaaacttgaagactgaattcatgggccagatacagttggggacagagattagcctatttgcaatggaacttttgaggtccttagaatggtcgtacatttttaaaatctttatattctccccagtgaggctcagtgacagaaaacaccaacatttacccaaacaactgccccgttcccctggtccttagtgagtcccttgagaaactgcattattgggataagatggaattaagataaaaactgagcagtaccaataaaaaatatgactttaaaagggcatgaaagcaaccgttatttagtatctgttattgctgagtacaatgtaatatagaacttgaacttgaattcaaacttacactcctccaaaacctgtattattttctccaggccctgccactagccagtgagggaaatagatgaaatagatctgactctaccttgaaggtctgcagcaggggtcagcaaacttctagccctcaggtcaatagcctgccgcctgtttttgtatggcccatgagctaagaatgatttgtacgtttttcaatgttttaaaaaattgaaaggagtataatgtttcataacacatggaaattatgtgcagctcaaatttcagtatccataaataaagttttattggaacacagctacgctcactcattagatattgtctatggctgtttttgtgcaaaatggcagagttgggttcagagttagcaacagagagcttgtagcctgcaagcctagagtatttactatctggatttctacagaaaaaaaaaaattattgccccctgccatacagtctgactgatagcctgagaagtatgcattaaaagaagttacctaccctgacaccatgagaatgaatttgaaaagaaccaagatgtggtagaggcagataggctatgaagtttcagaagggtagcatcactgtgggcaggatattcaagaaagacttcaaggaaaatgtggggtttgaactggtcttgagtaggagtagaacttaggggaactggtttgaggtaggcagctgtaaggtctgttcagagatcagtgagcagaacattcaagtatggacagaagtagcacagtgtgctttagaatataggatcatgggctgggtgtggtggctcacgtctgtaatcccagcactttgggaggctgaggcaggcagctcacttgaggtcaggagctcaagaccagcctgtccaacatggtaaaaccccatctctacttaaaatacaaaaattagccaggtgtggtggcacacacctgttaatcccagctactcgggacgctgaggcaggagaatcacttgaacccgggaggcaaaggttgcagtgagccaagatcgcgccactgcactccagcctgggtgacagaggctgttgttttagaaactctgtctcaaaaaaaaaaaaagatcatcgaagggtgttgccacaagctggttattatgatcttggtcaggcctcactcgcccatctgtaaaatggaaatgatgacaatagcaccctgctcctaggtaagatgaggactaaatgagataagcagtttatgagtgttagtcatgtgtagtaagaagtttataaacactagggatgattgttcatttaaatatggctagaaaaggacattggacagacttgagaagaggctttgagtgtgaggctatattatctagtacgcaggaggaagccactggaaaaaaaacaagaaacagcaccttggccaagcagtgtttcagaaagtcatgacttctaacaggctctggcatccccagatgccatcaaccagcaaattataaatctgttactttgtggtttacgaattccatctttgtatatctacgtctttgataagcacagtcaagctgtacagtaggcagatgttgttaacccattttagaggtgaggaagcagaggccagggaactgggtgatttgcacaaagttgcctgcccaggaagggacgtggaataaaggcaggacctaagcctggttctccgactcctgctccagtgttctctcctttcgactgcattgcttagagggcaaaatgcaaaatggagcctgccttttatcaccactaaaatatttgcacacatgcacacaggctatatcgcaggagaggttcttaatgccagaatgacctttgatctgattactcctagctctctctgtagaatgagaataagcccaagtctacagtaactgtagctaccattaacagcccagaaaggtcacggagaacttgagtaccaattttctcactggagtaaactgacccgtatgtgctagtggatcaagaagaacaaataaaagcttgcaaccccagtttctttgtccctctgcagcagagcatcacaacctaaactgatgcacattcttatttttttctgcgttaccaccctaagtgcatttgaatgtttccagcaagttactgcattcctaccacccctgaaagcattttccaaaaacatgttttagtttaagttgactggtggtttggcaaacaaaaggctggaatatggcacgatgactcaaatttaaattcaatgccttaaattaagggcatgctgttgccaagcccctgctgggccttgatgtgcatcagcatcctggccccttcacacaggttcctctgccacagtgaccttggattcacttaaggctgtacatgcagtggccttcctctgatggagggtggcctcggccacctgggctttaaactggtgctatctgggatggagaaaaggccaaccactgcagcagcaataaagccaaaagagcagagccacgggcccatttgaaaatgatgcacagagccgggcgtggtggctcacacctgtaatccgagcactctgggaggccacggcaggcggatcgcctgaggtcaggagttcgagaacagcctggtcaagatggtgaaaccccgtctctactaaaaatacaaaaattagctgggttgatggcacactgctatagtcccagctactctggaggctgaggcatgagaatcacttgaacccgggagccaagatcgtgccaccatcctgcaacctgggtaacagagggagactctgtctcaaaaaaagataaaaagaaaaaggaaaagaaatgatgcacagaagtgattgtgaccccacccttctgaaacttcatagccaactgcctctaccacatcttggtactggggtcattaactttgagggtacagggtgggcccttcaatggctggttaaactgttagtgatttcacaggaggaaatctgtatctcttggaatttctggccttgttagtcattattattagcattcagacaggcagatcctgcggtgtctaggtgcccacggaagtgggggacactcacagacggggggacatggccacagaattggagggggctgtgcagaaaaaggaggccacctgaaaaagtgggagatgcccacaccaccctagatgtagtagggtatttgaagaagtaggagggtacccacagaaattagggttcccatggaaatgggggaagactaagactagagcatagactcctaatagtataaaataacttaagcatgcactctcaatttatgcatattcataaattcacttataactcatatacctctaaaagatgccagtaatgatgtctaccctgcagtgttgctgtgagaatgaaattgaatttcaacctggtaaatgctgtagaaccgcgcctggtatgtggtaacattcaaggagtgtaatggttgttgctgttactgtcatcatcaactttagggaccttattgtggacattagaaaatacacaaaatcagagattaaagatgatataaaatactgagatttctgttttattatttttctacctgattttctttccagtaccacttcagagactgctggatcaaaagtgtgcccattttgtataatttttttttcttaatgactcaggatctaaacctataccaccagcatcccagcagtgttttctttcttttttttgtttgtttgttgttgttgttgttgttgttgttgtttgttttgttttgagacggagtctcaccctgtcaccaggctggagtgcagtgccgcgatcttggctcactgcaacctctgcctcctgggttcaagcgattctcctgcctcagcctcctgagtagctgggattacaggtgcccgccaccatgcccggctaatttatgtatttttagtagagacagggtttcaccatattggccaggatggtctctatctcttgaccttgtaatccacctgtcttggcctccgaaagtgctgagattacaggcatgagccaccgcacctggtcaatgttttctattaaaattctacctaagcatctaggctctgttgggggcttatcataacttaggatgtgccaaattgggcaggtgagctgggttttcagtgccagtcccacagaccttttctcatccaaggagggatgctcccaagagtatttatactgtgctctgagtatctgggacccagaatctgaaggcctcatgagtccactacaggaagaggtctctgatggcaaggagcggtaattgatggcatcctccttctaggtctccacatgcggcaaattgctttaaatgaatgcttttgagcccctgaacgggtatttctattttgggcccaagcacctagtgttgccatagccacttgagaatcgtgtcagacctagcccctgcttgtcaggtacttctgggctactctgggggacaaagtataactattcaaatggcaagttgaattagtacagtctaggagccttggagatggcttcttgaaagaggtagaacctgaaattctccttccttgagggacggccaggatttggccagatggaaaggcaagtggaaggctttgcagggacaagcaatgtaagcagaacctagaaatgggaaggaacaaaagtgaaggggaatgattgtgggtcaggggccaatggcgatgtgacattcagcctatagaaccatagggcagaggtgacagttgctggaatagatgggagctcagtttaaggaagatcgccaagccagacatcgctgttagatggatgtaatggggaataaactgccattgtcaactcaaaagtaaccagggccaggtgtgggggctcacacctgtaatcccaatactttgggaggccgaggcaggtggattacttgaggtgaggagtttgagaccagcctagccaacatggtgaaacaccgtctctcttaaaaatacaaaaatcagccggacatggtggcaggcacttgtagtcccagctactcagaaggctgaggcaggagaatcatttgaacccaaaaggcagaggttgcagtgagccagctcacactactgcactccagcctgggcagcagagtgagactccatctcaaaaaaaaaaaaaaaaaaaaaagtaaccaggatgggtaaaagatgaagaggcaagtctctctctgctgagaatacaattaatgcatgtaaacttggctctccttattattttaactccctctgttaaaagagagctgtttcacttgcaaataatatttatccaatcttgacatgcctgcagattattaatgtcgctgaaatttttttctctttcaacctgtcagtgttcaagctcaaagtcagaacaaaactttccaggttttttatctgtgttattcattgagtaaataagggtcttggaagtacccagcactcagagcagagagagacactgcctagtctaactaaaggtttattatagaagacgcaaccttcctatagatgggcacccaacttgggtattgagcatgtgaggcatggcaaggctgcttgaggctgggactcgtttctgcctttgataactctggcagattttccagtcagacacatacttcatactgtaacaccccactgttatacgagataaggaatttggggataaattgtgttctcattcttaacaattctgtatgagaatgatagagcatgcttaggcagcagccttgtggttttgtccttttaaactaacaggtagaggaagctgctatttttgttgatgcttctgaaagcccaagtgtttactgtccctccagatctcaggctgcttgcccttctggaaagtgtttgagatatgtttgcattataaataatttactcctaggcccatgccactcctggggcatcagtctggaactagtctccatatagatatcagcctgaccagccagatggtccaccattgaccttgatgtgtcatttggccatcatgagccacctcatctatagcaggaggaaattcgggtaaactgtttcctgagtcccttccagctacaacactctttgattcaaaaagacaaatgtatgttttaaaaaacactatttgttgagtgcttaccatgtgccaagctgggggtagagtgggaaacaggacaacacttgctgtcttgagggttccatcctaattagcctatcagcaagtaagtgaacaagaccaattcagatatgcataaatgcaacaatgaagaacaatgaaaccatagtaaggtgaagatggcaacagggaggtttttgcataagatggccagggaagggccctctgagcccggggcaggccacgtttgaactgagacctgagggatgggatggagccgagcatgtaaagatgtggcagaggcaaaggcccagaggtgagaataagcttggcctattcgagagacgaataacctgtgtagccggagcagagtgaatttgtgcaaaactaggctggaaaaggggtagaaaccagatcagggagaaccttgccagtcatggtaaggagcttgagttttttcctaagcgcaaggagaagccatttgaggattttaagcaaagaagtaacgtgaactgatggatttttaaaactatgaatgctatgctaaaataaactgtagggaagaagacgtaagaagcctgctcttgatacgagagcagtgagataagagggaaatctctcttctgcccattcgtcttgcacacaccacttcaaactttggttgatatccgctctgagatttgctatacgatattgcttgcaactagatttgtctcctattcgttgcctccagaactagcatgagccaaagctgaagggcaggatgtggtttcagcatgcggtgaccaccatccctccctccagtagctcagagccattcctggtactgcacggctcgtcactgcagatcatgccggatgcccagagtgcccacacgataatgacaccacggaattccagaggagccgagaagcctcctcctccttccccttccctccccctgcctctctttaaacatagccacaaattactagcacctgggctaaaatttgcccagtcaaaaacaagcagccacttccaaacaagacattttcctctgttctttttttctcttgtggctaggggcaaagaatccaattggctaagtcatcctggtacacagattgctagatacaaacctactttcctaggcacgcagctctcttaacacaaataaaatcgataaatttgggctgtgcgcgatggcttacgcctgtaatcccaacactttgggaggccaaagcaggcagatcacctgaggtcaggagctcaggaccaacctggccaacatggtgaaactcgatctctactaaaaatacaaaaattatccaggtgtggtggcgggtgcctgtaatcccagctactcagaaggctgaggcaggagaatcatttcaacctgggaggtggaggttgcaatgagccgaaatcacgccattacactccagcctgggcaacagagcaagactctgtctcaaaaattaaaaaatttaaaaatcggtaaattctgctctcttcactactagttccatacatcactacctagtagtagctctgggcctatagagtgatgctgatgtggaaagtagactctgtggtttacccaaagcagttgcctcatcctaaatgtgctggtagcactttctgagcattacagtgcccttaatattaaaagttgtttaccagtcacaacatattattatataaaatttcttttttttttttttttttgagatggagtcttgctctgtcgcccaggctggagtgcagtggtgtgatctcagctcactgcaagctctgcctcccgggttcacgccattctcctgcctcagcctcctgagtagctgggactacaggcacccgccaccgcatccggctaattttttgtatttttagtagagatggggtttcaccattagccaggatggtctcgatctcctcacctcgtgatccgcctgcctcagtctccccaagtgctgggattacaggcgtgagccgccacgcccagcccaatattatataaaattttctagactctgtttccccagaacataacaataaatggtattgctcccattttggagtaaaatttggccctccatgcaaattgattggccttgacaaactgaagtcctgaaactgtcagaatatatgttctgccaggcacagtggctcatgcctgtaatcccagcactttgcgaggctgaaacacgaggatcgcttgatcccacaagttcgaggccagcctgggtaactaggaagaccctgtctctacaaatagaaataaaaaaaattagccaggcatagtgctgtgcccctgtggtcccagctacttgggaggctgaggtgggaagatagcttgagcctcggaggtaaaggctacagtgagccatgactgtgccactgcactccagcctaggtgacagagtgagactttgtctcaacaaaaaaaaaagaaaatgtgttctctgttagacatatctgggctcaagtcctggctccagcatttgctactgctggtatggctttaggtgagtacttctctgagcctcaatatcctcaatggttaaatggagaagctatctcactgggttgctgtaaaagataaacaagattacaaatgtatagtgcttagctgcagttgctgacacatagtaagcaccccattgttattgttattgttagcaaaataagactccatggtagaaaaactatgcaagtcataaaatgtatcagcattaacttttatcaacagatagggtgctattaaatgtatcaccaacatagtaaaatgacagaaaggaatattacagtgtgaaagcatttatttatttgtttgacaacagctacaattgaaatacagcaattctgaaggtctttaaatgggttgcactttgccctgttaactcactatttcctgattggcaataattttacttggtaatgctctgtattatggtaaaagctaacaataataatttatcgagtatattctttgtagcaggcacagttatgagtgctatgtgaatacatttaatcttcacagcaactttgaagtaggtactattactgtgcatattttgaagatgggaaaactgaggcagacagtttgcctctggagccctcactcttaaactcttatgatatataccagaaactgtgacatagcccaggtgccaaatctggccctccacctgcttttgtaaataaagttttattggaatacacccatgcccactcatttacacattgtctatagccatttcggtgctatagcttcagggtggagtagttgctacagagaccacgtggcccacaaaccttaaaacacctactgtttggcactttgcagaaaaagtttgctaacccctgatgtcaactttaaacttcacaaaaagactggagtttaaagggaaagaaataatatcttataataaaaatgccttatgtttggtatttactgtggatttgtacatatttatcttacttaattctcatagcaatcctgtgaagtaagtagtctttttatttccattttagaaatgaagaatcaggagttcagagggattaaatgacttacactctaggctgaagagcaaactccatgagtgtagggatcatgtgtcttcatcatcatgtccctgtcacctaccacgatgctagcacagggtggacactcagaaaatacttgctaaatgaatgacagttacaaaggtcacccagctagagaatccaggtcttctcctcttccagggttctttctcctgactccacagctgtctgacagttaaaaaaaaaaaaaaaggtaggggggactttcccttgagataggatgaagcacagaatggttaacatgtcgttatgctgtcagttgacaaatttttgctgctatattcctccttctccttagagaagataatcagaccaaagacagttcgtttctcctccagtgcacagtcatagtgttgagagtttacttgtccttgtgattggtgcatattttgacaagattaaaactgacaccctcatgagatggtagtcacatggaatctgggttttggatggatttcaggcagagctctggctgccttcctcccaaccccttgccccacctcctacccagtgatgagtcacagtgcacagctgactgcccttcaaaagccgacccgaggcttttgttttctttcatctgcttcatgttggtcatgtgaggatcagggtattaatagaccatcatcttggccttccgaatcttgctgttggtcagggaggtaaatgaaaggtttggccagtccctttgtgccattttcactcctctcttgactgtcacgtgctggttggtggcaaagctaaaccacagcattcggtgtggtcaccgcccatggccatggtctctactggtgttttgatttcccgcctctttcagcagacactcggtgagctcctgacttccaactagatcaccagtactaattgcaacaaaccacctcctcctcccagtcagctagcctcatctctccctgcatgtggaaaagatacagaagccagggagaaaaaggagtttggtttgggggtggctttttttaatatgtaaagtaaaattacattcaggaaattaaaagtctgtgtgtggtttaacaggctcctaaagagctccacggggacaaacatgttatgcaaatgccatggaagcagaggtgctctgaattgcctaggccgttgatccaaaagtttccattctcctctgcaacccaatctttaaaatttcattttgcttcttccctcagccttgattccagcacccaccacagtgtcatgctcagtgtttattgatctgaaatgaagctgggctgaggcctggccaggagatgaaactctcttagaaagtttcctaggagcggagattctagcttcagtgtaaaggaggggtctcggttctctgtgtctagtctactgagtctcaggctagttattaccacaattacttttgcaccagcctaatagttttctctgggggcctggtgggttctcgtgaagttaattactttcactttatatcaacctcttttgtttgttaactctttgaaagcatgaagtgtgatattttcctgtgttgagaaggactctgctgagtcctcactagttgtggagtggctcataaggtgggtctcttgagcctcgctttgctcatctgttaagtggattaaactagatggtctcttaagattccttccagtctaaagtttcatttttcaacaataggatctttgttttttattttttattttttaagatggagtcttgctctgttgcccaggcgagagttcaatggcacgatctcggctcactgcaacctctgcctcctggcttcaagcaattctcctgcctcagcctcctgagtagctgggagcacaggcatgtgccaccatgcccggctaattttttgtacttttagtagagatggagtttcacatgttggccaggctggtcttgaactcttgacctcgtgatctacctgccttggcctcccaaagtgctgggattacacgcatgagccaccgcacctggcctttatttgttttttaatcttgccatgtcagtaaagaattttgctatagaaggtaaaaaagattggactaagtacgcactaacttcagttattatcttcatttatttttgtctggcaattatggggttggtaggaatgggacatactttttcaacaaacaaactggcggggcgcggtggctcatgcctgtaatcccagcattttgggaagccaaggcaggtggatcacctgaggtcaggagttcgagagcagcctggccaacgtggcaaaaccctgtctccaataaaaatacaaaaaaaaaaaaaaactgttagccaggcgtagtggcacatgcctgtaatcccagctactcaggaggctgaggcaggagaatcacttgaacctgggaggcggaggttgcagtaagctgagattgcaccattgcactccagcctgggcaacagagcaagatgccgtctcaaaaaacaaaacaaagaaaaccctttatttagcacctaattagtgccaggtacttgctgagggctttatgtgcattctcctattatcactcacaacaaccttgtattattgactctaccttgtattaaaggtattaaatactcaaccttgtattaaagactctaaatattattagtatagatgaacaaactgggtggggcaccaagaaatatagtaacttgcccatcatcacggagctaatacatgttcaaatcaagacctgaatctaaggccaagagctacaaggatccaagtttttagccacagtgacatcatataaaatacatccctgaatttctctctcttcctgtgattggcgtctttctacagaaatgtaagtgcagtgaaagagtctaattaatagacatgtttattgaacactgcagaagcaggaggtatcaggataagactctccacttccatggaaaaagtgcacatggctgctgaagatgggctttaggtgccttgagagcagtcagtgccaataaggaagaagttcaagcgggggcagaagaattcccccgggaggtgattctgcagaactcagccattcacaggtcatgggagattttgttcccttcagggtacgtcagcttcacttgctgaagtaggacaagtagattgaattagccctggctgaagccaaaattctttatatttaaaagaagaaaagcaattaaatattcaacccaatcctgggttttgaattaccccatttatctttcactctgagcatctgctttttattgcattgtggccctgcctgccatttatctctccccgtcagtctgtatccacgtgtcatgggactcaaaagtgaagattagaggagaaaaatatctctgtactctaagcctggcaacttctatttttatcctcagctgttggagctgatagcaaagtcacagctcacatccctgagtggcatcgcccaaaagaacttcatgaatattttggaaaaagtggtactgaaaggtgagccttctctcactctctcgcccctttttataggcatggaggtgggcagatggattttccaatgaagtggacgtgtcattagacttaaagacatgtgaatggatggaaatgaataactccagctacattttagagacactaaattccagttcggaaaggggtacaactcatcctcatggcaaagggtgagaccatcaccctcctgtcctttctttgggaccatccacttgccctactataagcctcgtaggcttctcagtgtagacacaggtcccaacactggctaccgagcctccgggtcccaaattcctagaaaatcgacatgtgtacaaagtcagaatagattcgtagactattcgagctagaaggaaccttaggtctctggttcaactgactcattgtacacatggtgaaactgaggcccatgaggagaagtgatttgtccataatcacacagagagtcagccagacaggattagaacccaggctccaaacccctgaaaccatgctccttccactgactgtcctaaacggggtcacttctgggcaaaaggagtggctttctgatcaggtgaaccatggctgctgggagaacaatgaaaagagaaagaggctttatagaaaatgagagagaagagaacaaagagggacctagataatggaaactctaccctttaacctcaagtgcagaagcttggccaagcgtacaataaacactggtgaggaagaaaaccatatccccagaggcagtgcctgggtaacatgagtggaatacagacagagacaagctcatgtgtaaaatacagggtgttccagtatcataactttaattatttttttcttccctactcaagtccttgaagaccagcaaaacattagactaataagggaactactccagaccctctacacatccttatgtacactggtccaaagagtcggcaagtctgtgctggtcgggaacattaacatgtgggtgtatcggatggagacgattctccactggcagcagcagctgaacaacattcagatcaccagggtaagcgggcaggcccagcctcctcccggcagtgggtccctccacagggatactggacagactcgccaagactttgaattcactcctgtgacggaagagtctggtcttttctgatctgctgctttgttctggtgctgagggtgggctgggggacctcctgataaagggaagtgggaagaaaaacttggcaaaacgggatcaaagtacatgaaagatgacagcctggtgaactattatgggaaagctggaaacttggcttttggggagatgctggccatcctgctgaaatctttagcggggttcagagatgtctctttgctcatttcctcctaaagcatgagttgtttgcagccttaggttgctgtcttgaatacagtagagctgggaaggcagtagcttggagaactggtaccagccttttaaaatcctagttccgattattggtagttatgacaaaggaagcctttgattaaagtctgtgaacttacatgtgggaaaatgatgagcaattctcaaagcaaaaaggaaaagccgtatattaccagtgaggtttggcgatctatagcagttgtttcttttggctttcacaacatagcttggccctcagctcttgtcctcctaatagtgagcaggaggaccaaggcacaagaaaaaaaaaaaaaaaaaaagggaggaagacgctggtgtaatctcttagggctcactaaaggacagacacactcaggcggctcatgtagcactttgcttatttatctcatcactgactgtaactggcttaagtggtttccgggacccacactgataaagtttgaggttgcctcatatcaggagaaggtagcaggaagaaatcctttggcttcttttaaataactgttttactagatgaagttttcaaaatgcatgttcctgactttactggtaattacgtctctgtacctgcactttttaagctgtgaaacagaatcattcgaaggctcagaggtatgtgacttccttagagccacttaggctacaacacctacacgctgcacccacagacacacacacagagcagagcacggatgcaagtcatctttcagaaatgatctcaagtatttgatttttttaagagaatgtacatatttattatgaacatttacctgatttattctaataatttaccccattcccacatctcaagtgtaggcagatagtctgaccatgtgaatgtgagacccaagttcccaacttatctctcacagaaaacccacatacacattctacctggtttctggcattctctatggcctggactgactgactgggatttttctaaggccaggagagaactgtccagccagtgatataaacaagcgggtcccatgtgtcccaagggcacttcccttcagcaacttcagaacacccatttagacgatcacagttggccaggtgcagtggctcatgtctgtaatcccagcactttgggaggccgaggcaggcagatcacttgaggtcaggagttcgagaccagcctgaccaacattgtgaaactccatctctactaaacatacaaaaattagccaggcatggtggtacacacctgtagtcccagctactcgggaggctgaggcaggagaatcacttgaacctgggagttgaaggttgcagtaagccaagatcactgctcaccagcctgggtgacagactgagactccatctcaaggaaaaaaaaaaaaaacaaaaaacctctagatggtcacatttgcctggagcttgcctctgttccctctgtccacttccaaggacccctcctggccacatctgtccacataccctgcactgccataggctcacatgctcagaaccctccttagctcatccacaaacactcttcggagctagctattgtagctgttgttacccctcttggagtggaggtggagtggggaccagagacacccagtgactttcttagcgatgcacagcacttagggccagagctgaggccagaaccttggattccgtattcctaaaatggcaaagcttgcttgacatcagtggtttccaaactttttcaatccagctttcttccaactatttatccaatgatccagctttttaaaaaactctctttaatccaatgagatcccatgaagaagagatcaaacactgaagttcactcactatctcttctgcacgaggagctccagccgagccaagccattctctgcacactccaagcttggccctgcctcagggctttgaagcttgctgccttctttgaccatccaatctatggacatcaccatcatatcaccttcttccgagcagctatgatctgaaattgtctaatgtgtgtgcacttttctgtctctgtccatgggcttataaactccacaaagaaaggacattacccatctattccctgttcctggcacagagccggagacagtcagtaaatctgagctgaatgaatgaatggttgaaagggagtctcctcccattgcctgcctgagtactcgttcagtgttcaaaggaacacaattggaaaacatggccctgtattatgaatgatgataataatttaccagctgtaggatttctttttttgaaacagggtctctgtcacccaggctggagtgcagtggtgtgatctcagctcactgcagcctccacctcctgggttcaggtgattcttctaccttagactccccggtagctaggattacaggcatgcaccaccacacccggctaatttttgtattttttggtagagacagggtttcaccatgttggccaggctggtctcgaactcctgacctcaagtgatccgcctgtctcagcctcccaaagtgctgggattacaggcatgagccaccgcgcccggccccaactgcaggatttgaggcaagttattcagtctctccatgcctcactttcctcatttggaaaatagttgcaatagtgtcaactcatagccatggtatgagatttaataagttaacatatagaaagcagacggcacagtgcctaatgcacagtaagtattgtaaatgtatcagctgttattattaagggctggctctgtgcaggccttcttctaagtcctttgcatgtattaattaactcacctagtgaagaattgtaaataggagtcatcacagtggctagcatttattgggcgctgtactgagtgcttctatgtattatctcatctgccccacccagcaacaatgtaggataagctttttctcatgttataaatgagaacaattagaacaagtagttaagaataatttggctgatgcctcatgcttagtaggatgtggcacaagaactgaacccaggcctgtccacagaggctgtgctttcaaccactgtaccttcctcccactgtcctctgtcccaaccctccttggctctgagcttccttatattgagagctgtactggaacccacggagccttaaagggcttttctttttttgtttttgtttgtttgtttgtttgtttgtttttgagatagggtcttattccccttgcccaagctagagtgcagtggcatggtcctaggtcactgcagccttggcctcctgggctcacatgattctcccacctcagcctcccgagtagctgggactacagtcactcgccaccacacccagctaatttttgtatttttagtagggatggcgtttcaccatcttgcccaggctgatctcgaacttttgggctcaagcaatcctcccgcttcagcctcccaaagtaccaggattacaggcatgagccactgagcccagcctccacacagaagctcaactctgcagaggaaagagaaattgagagagagagaaagaaaaaagacttaattacttcagtctggactgtattttaaaacttacttttttaacagtttggcccaaagaaaagtacattattataggcaaatataaagtcaactttcagagtaaatgctttctattttgccaaagttctgtcagtctccttccagcataaaattttgttttcctgcaaaatttttatatctaattctatggagccaatttttattggcaaaataattaatataataaagaatgaaggaaacaaattaagagaactgggtcttggggaaaaaatcaaatacagtgacagtatgcggtgggcagggcactgttttgaagcatgtgaggagtgacatagaccctagtggatgttagccaaatgcttgactagaggagcgagagtgactaatttttcttttatacgtttatattcgaaaggaagaggcatgtaagcagttaaccacttatcaggaggaagggcttagcatggggtctgggcaaggattttggaaaagttcaggtttgaaaatttgacagagtgaaccctgccaagtggacttgcctttaataggagttaatgtaataattggcctgggagttttaaccaaaaaagtagaggctttgaagtgttggaaacgaaaagaaaatctcatggcagttttattagctcatatgatcagcaggagatgataaacgataaataggtctagggccttctgcggttggcaaagacctgaagtttgcaaacaattgagagctctgcccagcagcctggggtagaaccgagcggtataaattgtcaagtcatagatactttgaatgaaataggaggctgaagtccttctacatctgagattgttggagctggaagggatcttcagagtcttctaagccattcattttggacatggggagacttcccaggggagctgggatccagatgtgctgcctgtccatccatgttgctcccctgcacctcccattcttctccatagcttagccttcccaaggccccttgccactgctccctccccacctcgtgcctaccctgccttgctggggacacaggagagaactgcccagacctagtgtctcctggaatcctccatgtgtttaggcttcgatgtgctttgtacagagtttggctcctgtaaaaatttgggcagggaaattgatacccattctcaggatattttcccctgagctaaggaagggtttcttcatcctgtattatttttcccctgtgacctctcagaggtcacagaacattgatggaaaaatgtgagtagtccaaaatcaagagctcacccagaaccagcaggggcaggacaaggaaccgcagtcccaggtggtctattgacttatctggatggcagcatcctgttgctgttcaagacacatcagcaaatacatatgcggattcccaggtgcagattcttcccacagcgctttgtgcttccctccatgacagcgtttactgtattatttcatattattttatgattatttgtttctgcctctgggtctctcaccagcctgaagagctcagtgcagccacttcagcaccaggccatagtaggcactcaggagaggtggcttgtgctgggctagtctacaagggacataaaattagaccctgccttcaaggatgtagatctgggcagggtatcatgagctcatcaaaatttaaaacaataaaggcttaaatatattaataagttcagggatgtaccagaagctcttggagaagctcgccattaattgccaaggtatcatccacagaccagcagttttcacaatgctccacggaggcctcggttgaaatagagaagtctcaagggatggtggagcttgggccaaaggcttctggacctgccattcccaatgtgcacgtacacacacacacacacacacacacacacacacacacacacaccctagcacagagtctctgtgttattgttttctacctatttaattatttgggcttcattcattccacaggtatttgttgagcaccttctgtgtgctgggcactcttgtagacaccctggacacagtagtgaaaaaagattaaaattccttccttcatagagcttaagttccagcaagaatgttcctttcaaagtttcttttagaaaaaaagctcatgttgcatgacaaaaagaaggatattgcaaaatgtgtctcaaagagatcctaagaattctaggaaggtgcagtgagtaagggagtcacaaaaacagaaaaggcctttcagaggaagtaggtttgaactggatctgagcaggatgaccacgtggccgggcagagaagactggggaggcgaaggcattgaggtcaggctgagccaggtgtgtttaggtgccagtgagtaaacctacttggagggacctgatgtctggaggggggccagagagatgggacttggggtggcaggctgggccactccatccagggcagtgaggccttcaggaaagcagcgaaccttgtaattgtgtgaactgagtaaagcacatgcgctatttggagagtctttaagagtgaaagtttcaagctttggaaatgtagggcgatccctctggcaccactcagctcagcgcactccacacttcattgagggtcatggccatggtctcctgtctgcatttaggatggggagatttactactgctttcgacctcttatcaataaactgccttgagggtggaggacgggcacagctgcatgcctggagggtgtggcctggtgaatcgctgctctctggcccccccacctattgtcacccttctctctgcagcctgccttcaaaggcctcaccttcactgacctgcctttgtgcctacaactgaacatcatgcagaggctgagcgacgggcgggacctggtcagcctgggccaggctgcccccgacctgcacgtgctcagcgaagaccggctgctgtggaagaaactctgccagtaccacttctccgagcggcaggtcagcaaggcccagtgggctttcctcccaccctccttctctggccctcaccccaaacttccttggtgccctgctcaagcctgaggtctgggttccccctattccaggttccctggtttgactgaaaaatgacagttgaaggagttttgtgattttgttttttgagacagggtctcgctctgtggcccaggctggagtgcagtgatgcaatcatagctcactgtaacctcagccacttgggctcaagcaaccctcccacctcagcctcccgagtagctgggactacaggctcatgccacaacacccggataatttttttattattgtttttgtagaaatagggtctctctatgttgcccaggctgacctcaaactcctggcctcaagcagtcctcctgcctcggcacccccaaagctctgggattacaggcatgagtccccatgcccaccctgtctgagtttttaaatagagatgttgtgtgtgtgtgtgtttcaggtagtttttatggaaaaaattaaacttacattcttaacctattctcattttacaactcactcaaaaaaatcataaattggcacccatcctttttttctataacacaataacttacattttctgagtgctttacagtactttttgttttgttttgttttttgagatggaatctcactgtgtcacccaggctggagtacagtggtgcagtttcagctcactgcaacctctgcctccctggttcaaatgattctcctgcctcagcctcccaagtagctgggattacaggtgcacaccgccatgcctggctaatttttgtatttttagtagagacagggtttcaccacgttggccaggctggtcttgaactcctgacctcaggtgatccgcctgcctcggcctcccaaagttctgggattacaggcgtgagccaccgcaccctgtgtggtagagtacttttacagatatcatttcattttattctgatggcccagggaaggataggcattgttgtgcccactttaaacagatgaagggactaaagccacaaatggctaagtgacatatccaaattagtgaagtagctctttgggtgaggaaactggccctgaccttgaatttgggccttcagacaccaactccaagcctgtttcccttcatcatgctgcctcaagtttgttggtctttttccagtttctttatatggatgtgtgagttcaacaagttctgttcgtgaagccactaatatttagatataaaatagagccctggtgccaagcagttattgcatctccctggaaatgatcactcaagtctacagcaaagaaaagattaaagttgtttactgagatcacctaaactttaaaggaccatccagccacagttgagtaaaggtcactagtaacatgtcaccagccacctggtttttttgttgttgttgttgttgttttttaagcttggttaaaagctactgtttaactgaggagagctaagcaggtacttctttatatagtgacagttttccttcctggctattgtagagctgaaaagagggggaaagggtgagagtaaaccttttcagaaccggaaaaccacaagccatcttcttgacaaactctcctgtcatttcctttattcatttgattttttagatccgcaaacgattaattctgtcagacaaagggcagctggattggaagaagatgtatttcaaacttgtccgatgttacccaaggaaagagcagtatggagatacccttcagctctgcaaacactgtcacatcctttcctggaaggtatgtgtctcagaggtggctgccacagacccccagcccagcccctaagagccaagagtgccagccagcatggtgaggccagcgcctttccgcctcttccccatcacagcccccagctgaaagtgagggtaaactgattcagtgcctgggagggtgggggggaaggacttttatagacctccattttcttcactttcttatgtttacttttttctgcgttccacccactcccccttaaatcctcctctctctgtgtgtacgggtacacacacttacacatggaaacacatagcttttagtttttccagacttataaaattttttcattgatgcataatagatgtacgtagtttcagggtccatgcgataatgtaattcatgtaatttgtcaagattaaatcaatgtactttgacacattcataaccttaaatatttgtcacttatttatgctagaaccattccagttcttctcttctggctattttgaaatgttcagtagatttttttttttttttttttgagacggagtctctctcactctgtcacccaggttggagtgcagtggtgggacctcggctcattgcaacctccacctcccaggttcaagcaattctcctgcttcagcctccccgagtatctgggactacaggcacatgccaccacacccaggctaatttctgtaatttttagtagaatggggttttgccatgttggccaggctggtcttgaacttttgacttcgagtgatccacctgccttggcctcccaaagtgctgggattacaggtgtgagccatcgtgcccagcctgaaatattcaatagattattatatactacattcacttactgatctaacagcaggtcttattccttctatcataccgtgtatttgtacccattgatccacttctctgcatccccttccttttccctttcccggctctggtaaccaccagtctactctctgtcttcatgaaatctactttcacagctcccacatacgagtgagaacatgcaacatttaggcctggggcattattgacagagaattgttgctttgaagttggaaaatgccttatggtgctaaaagccaaagagaggcctctgagaacttgaagctaaccatgtgttcctttccagggcactgaccatccgtgcactgccaataacccagagagctgctccgtttcactttcaccccaggactttatcaacttgttcaagttctgaatcccagcacatgacaacacttcagaagggtccccctgctgactggagagctgggaatatggcatttggacacttcatttgtaaatagtgtacattttaaacattggctcgaaacttcagagataagtcatggagaggacattggaggggagaaatgcagttgctgactgggaatttaagaatgtgaacttctcactagaattggtatggaaaagcaaaatactgtaaataaactttttttctaacaatttgccag

SEQ ID NO: 2 - protein, 355 aa, Homo sapiens
MPFLGQDWRSPGQNWVKTADGWKRFLDEKSGSFVSDLSSYCNKEVYNKENLFNSLNYDVAAKKRKKDMLNSKTKTQYFHQEKWIYVHKGSTKERHGYCTLGEAFNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETILHWQQQLNNIQITRPAFKGLTFTDLPLCLQLNIMQRLSDGRDLVSLGQAAPDLHVLSEDRLLWKKLCQYHFSERQIRKRLILSDKGQLDWKKMYFKLVRCYPRKEQYGDTLQLCKHCHILSWKGTDHPCTANNPESCSVSLSPQDFINLFKF

SEQ ID NO: 3 - DNA, 76939 bp, Homo sapiens
aaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaaggtaacgcgccgctgcgggcggcccggccggcggggctccgggagtgcgaaccgggcggcggcggcggcgccaggacctccccgccactgctgtgccggtcccgggtatcgccgagcggggctcaccggggcgccgcgtttgtaggcgtgcggggggtggagggtgaggggagagcccccctccccggaaggagctgtgagcttcgggctggcccgcgggaccctgcgagtgaggcagcgcacacccctggagctgcggggcggggggcgtgtggggggctcctttcccggtgcttgtcctggccggggcgccggggccggggtcggggcgttccggtgcccggtccagggtggcagctcgagcctcaagtctcctttgtgtggcgcggccgcggcgggagcgcccgggtgggacgggacagacacaagttggtgggacccagccgccgagtccgcactcagacaaaggacggactgtgtctccggagggctcggcgcgaggggaaggggcatgacacccgggcccgccggcgggcgggagaggggcgtggggaggcgcgggccgctgccggggtgggcagggctcggccctcgcctcgcgcacgcccccggtcccgggcgcgccgaccccgggctggggccgcggcgctcgcgggacgacgttcgcggcggggaactcggagtagcttcgcctctgacgtttccccacgacgcaccccgaaatccccctgagctccggcggtcgcgggctgccctcgccgcctggtctggctttatgctaagtttgagggaagagtcgagctgctctgctctctattgattgtgtttctggagggcgtcctgttgaattcccacttcattgtgtacatccccttccgttccccccaaaaatctgtgccacagggttactttttgaaagcgggaggaatcgagaagcacgatcttttggaaaacttggtgaacgcctagtgagttgccactttctaaagaatcgtaatccttaaacattacaagctgttttggccaacttgtgtgatacatctgagaatggcgacaggtgctaacgaaatcaactgtcattcagccagattattccccaaatcccgacgggttgaatgaggatgaggtggtagaagtaggtgatcaaaatagaatggctatgacaaatgtgtgtttagattatacggttagcagtttcttattaattggtctcgttttctgtgaacaatagctttggtgattcagaaaagactgcatggagaaatgtttgttgcctaagaaatttaacgtgtgctgtccacagttaaacttcctttatcacacaggatctgtaacatttaaattcatttggaaataaaaatgtgtctttgctatttacttaaaggcttgctttttagggttcaagcaagttattattagtttaattttgtggcgttttgatgtcatcataaaacagcatttaataaacctttgaaaaacacgaatgctgtcacaattgaatgtagtttatacttttaggggttttggagtttattttctattactcttcataagcattagcaactaattcatgtttaggaatactgtacaagtgtgtaatcctaacttgtttatttgtaattttatttgcaggaaaacaagtatttaagatatattttaattttctattcatggtctctaaagttaggaataggtaggattctagtcatagatataaagcgtcttgttctctcttttcggaggtgtgttgggtgatgtaatatttaaggtttcccggaaacaggaacactgcatcttaaattctcgtttttgtggaaagtgaatatattccaagaaaacaagttagaaaaacataggcgttcttctggttgcactgtcctatatgataaagatctaatgtgcatctgctaacttaaggtttcataaaaatggagacaaataaatgcaatataaatttattacatatattttggagagtgttgacaccactgtacgggcattccaggtgctggtctaagtgttgagacattataacaaaaaagtaacgtttatattagttactattaattctgtatttaaaaaatcctattaataatcttgtccattatgagataaggtagttatgcagatttttggcagaaattcggatattttaggatacatatgtgtgtgtgtgtgtgtgtgtgtgtgtgcgcgcgcgtgtgtgtgtgtgtatatgtttttttttttttgagacagagccttgctctgtcgtccaggctggagtgcagtggctggatctgggctcactacaatctctgcctcccgggtttaaggaattcccctgcctcagcctcccgagtagctggaactacaggcacgcaccacgacacccggctaatttttgtatttttagcagagacagggtttcaccatgttggccaggctggtctcgaactcctgacctgaagtgatgtacccgcctccgcctcccaaaatgctgatccagacgtgagccaccacgcccagcctaggataaaaatttctaactttgcccttgagtgttctctgtatttgataattactattctttgtacttgccatgagatattttgaaaaacatctctttggggaattggtttaaaaattcatattaaattattttctggtacaattgagaatgtatttagtcagtttaagtagcagtgacttaaaacacacttgttgatctagtgtagttagaaaaagttctgatactttgatacctagctttgccttgcacaaatacatttaagtgaatatctttatgttggccatttaaaaattttaggcaaatttctccaagtcatactggaattttcccaggataccttccttcttttgaggtagtgtgctcaaggattctgaatttatttcagtattaccctttacatgagcagtgattgagaaagttgatctgctttttaaaaaccaacaaggacaaatcctccaatataatgtattctgtggttaattagcaatgatttatggattagccttgaagtctttatatcagtatagatgacaaattttctgtaaatcatgatagattctatatgagtaggtgaaatcttcgggggagagcataagtgaacagcagcaattcattaaattcagagaaaggttcatggtcagatgatacatactttaccttctggaggtcttccctgaattcccactcttgtgttcccacagggttttatcatattttattgtagcatgactcaattgtattgtaatcacttgtttatgcatccctctcttccctacagtcaaagtgcctcctggtagaggctctgtcttatttttttctttgtatacctagtacctacgttggtatttgctagagaataggggttttaaatatagttgaatgaattaaggaaatatgaaaaatgtagggaaataatttgacaaaggaacttgcaaagtgaacaggatatgttagtattcatgtgaggaactttgaaatgtgtttttctccctctttaatagggaatgattacaaagaaaaatagttgtcaaggtgaacaagttccaagtttttcctagttataatctgtagtactaaacattagatatcctttagatgcaactattacactcggacaactggagagaacttgatgaatcatctggatatttagtaatagtatttgcttaatttgatttaatgttgatattgatgatctctttgcaaaaatataaggaagggaaagggcgagagagggtagtagatcttttaactgaaaagctgttttgttataattcatgagttctttattggagtaaattgaggtaggttttggagctaaaactgaaggatgtagcgacagtgtaattggatcatttattgggatgcttttcatcattcttttaaaatgatttaaatacagattagcaaatttagtgatacctcaaaagttacagctggtaaatttctaagatagttgcaccattccatatcttgccactgttccactacttggtataaaaatatgacaaatttgtgtttagattatgtggttagcagtttcttaataattggtcttgttttccattaacaatagcaagacagaacaagatcatagctcactgcatccttaaactcctgggctcaagggatcctcctgtctcagcctcctgagtaactggtattaccagtgcacaccattgtggtgacttatttaattttttaattttttgtagagatgggatcctgctttgttgcccacgttgatctcaaactcctggctccaggcaatcctcctgccttggcctcttaaagtacagggatgacaggctgagccattgtgtttgacccagatactttttttttttgagacggagtctcgaaagtgattcttgtgcctcagcctcctgagtagctgggactacaagcgtgcgccaccacgctcggctaatttatttatttattatttattatttatttttttgagatgcagtgtcactctgtcacctagactggagtgcagtggcaccatcttggctcactgcaacctccacctcctgggttcaaatgattctcctgcctcacctctcaagtagttgggattacaggcatgcaccaccaggcctggctaatttttgtatttttagtagtgatgggatttcaccatgttggccaggctggtctcgaactcccgacctcaggtgatccacctgccctggcctcccaaagtactaggattacaggcgtcagtcactgtgcccagcctccgataatttttatattttagtagaaactgggtttcactgtgttggccaggctggtctggaactcctgacctcaagtgattgacctgcctcgtcctcccaaagtggtgggattataggtgtgagccaccacaccctgctgtgttgtttttgtttttgttttttgtttttgtttttgttttttgatatggagtctcacacttgttgcccaggctggagtgcagtggcgtgatctcggctcactgcaacctctgcctcctgggttcaagcgattctcctacctcagcctcccgagtagctgggattacgggaacgcaccaccacacctggctaatttttgtatttttagttgagatggggtttcaccacgttggccaagctagtctcgaactgctgacctcagatgatccgcctgcctcggcctcccaaagtgctgggattacaggcgtgagctactgcgcacgcctggcctggatactttttaaacatttttaaattaaaaatttttttttggagacagggtcttgttgtcacccaggctggagtgcagatgcgtgatctcagttcactgcaacctctgcctcctgggctcaagcgatccttccatctcagcctcccaagtagctgggactacaagtgcatgccaccacgcctggctaatttttgtattttttgtagagacagggttttgccatgttgcccaggctggtcttgaactcctgaactcaagagatccacctacctcagcctcccaaagtgctgggattataggtgtgacccactgcacccagcttttgtttttaatatttgtaagaaccaccttgtgtctgccaacaggcgaattaacaaaatgtggtatataaaccaatgaaaacataaaaggagtgaaataaagacgtaaaaggagtgaaatttcaaaataaaagtaaaaagatttcaaaataattcagacataaaaggagtgaaattctgatacatggatggagctggagaatattatgcttagtgaaataagccagatacaaccttttgtacaaaaatacaaaattgtatgatctcatttatatgaggtagttagaagaggcaactctatggagacagaaagtagaatagaggttaccagggctgtgaggggagaggtgaatggggagtttaatgaatacagagtttctgtttggaatggtgaaaaaattctggagatggataatagtgatggttgaacaatattttgaatatatttaatgccacagaattgtacacttaaaaatggttaaaatggtaaattttacacgatatgtatctgtatctatatatatctctctctatatctatatatcttaccagaatacaaaatttaataacacactccgaaaacctttacagatgaggaaactgaagaaaactgtctacaggggaggagttaagaatttgcccaggattattcagctgggaatttgcattcgggatccaaacttagttctgtttcactacatattatctactccatattatctgttctgtgttatctgctggctttctgggtgattaaagatatgtcagctccgagaagaatgagtttatttgaatcattcagaaagttacatttaaaagtaggtaattgtagtttgatggaaggtacagtgtgaaaccctagacagactaaaggttaactttgaggatttctttctcagccagagtggtaatagtatgcatttgagaggggaggagagtagagttctaaggatgtggtctttggagacagtttcttgggttccagtccctgagctaccaatttgtgtctggggtgttatcctcttgatgtcttagcatccctatctgtaaattggtgaggataatgataacatctgataaggtggttgtgaggattaaaggaattgatacatgtgaaatccttagaactgtacctggcaaaaagtgtttgataaatgattttcagttattgtgccaatattattttagagttgatgtactttctcattaatggaaccaaacacttctcaagttaaaattacgtgcttaggactggtaagttacaaaaatggtaccacacgttttatctatttcaatttagaaatgtctgttgattaaatgtgttcgctttaaactactgaaacaatgtagacatttataaaatgaaagcgtattgatccctgttatctcattcgctacctttaacggtttggtgtatattcttccccaaattttcaaatatattcatatatgaatatgtatttttacatacattttataaaaatgggaccaagttatttggttctaacatggcttttttttaaggtcaatacaaagatctgttttattaaaaaataattgatattcctttagggctcactatatgcttggtactcttctaagtcattattttatatagatactataatatcgagagatggagagattaagtaacaactagttagtggtagaggaaggattttaatttgggtacgttagcttcaaagtcctgatctcccagccagggatcatttttggtaaggcctgtgagctggaaacgttaacacttttaaaagagttgtaaaacaataccaccccacttccctgaagaacatataagggagactagataccgcctgccaagcctaaaatacttaccatgtggccctttacagagaaagtttgctgccccttgctctaagccatccagctgtacctctttggtgtaaggggggtgcatagtattccagtttatgaatgtgcattacgcagcaaaccaatctgttgtgattgacattgttttctctcctgaaaagaagtgaacatccttatgtatctttgaacatttgtgtgacaattttctatagagttggctctttcaagattatgaacatttctagttttaataggtgttgtcaagttatattaatttttagttaaaacaacaactgtattgaagtataatttacatacaataaaaagcacacatttgaagggtatgatttgaggagttttgacaaatgtatgcacctgcaccgctgcctggatcaagatctataatggttgccatcatctcagagtcctttcatcctcttttacagtcattctctcaacttttttttttttccctccaagatggagtcttgctctgtcacccaggctggagtgcaatggcatgatctcggctcactgcaacctccgcctcctgggttcaagcaattctcctgcctcagtctcccgagtagctgggattacaggcgtctgccaccacacccagctaatttttgtagttttaggcgagatctcagctcactgcaaccttgacctcctgggctcaatcgaacctctcacctcagcctcccaagtagctaggaccacaggcatgtaccaccatgcccagctaacatttattattaatatttttttgtagagatggggttttcctgtgtcgcccaggatggtttccaactcctgggctcaaatgattctgccttggcctcccaaagtgttgggattacaggcatgagccgcggcacctgacttgtagtaaactctctgaattaatattccattgtaggcatgtgctacagtttttaaattcatttacccatggatggacacataggactgttgtcagctgttgataaagctgctatcaccatttgtatgtctttcctggacatgttttagtggtaaatattgattttactttgtaagaaaccgttaaactcttttccaaaatagttgtaccattttaaattgaaagttacagttgtaactgtgcaggagttacagtttcttcacattttcattgacacttcgtgttgccagtcttttaaattttggccatcaaatgagtattaagtatctcattgtgggtttgtgtttctcagatgatcaatgatgttggaacatcttttcatatgcttattggccatttgtgtactttttttggttcaagccttttgtccctttaaaaaattggattgtttgtctggttgagtggcaagaggtctttatatgttctgggtacatagtcacattatctgtcagattgtgttgcaaatattttattgttcatttttgtttgattttgtgtatttttaatactataaagatcaagttaaaactttaatatgggaagcataatcagataaattatgtgaaacaaattgtccttaattcacgagtcatttaattagtgtaacaaaatgttatgcatttgcagaaacttgtaaactaaaaggatattattcatatgctgttaggtgtatggatgataattttttttaattaaactagttttgaaaattattgtatttagtaattctcttcattttgcataattcaaaccttttcatttattagtgagttaagccttaaattttttcttcaaaggataaatgagaatattaaaagtaaaaagtgaccttgatcttagaatggggtatgtagaaatgatgattgccaaacttagtttccctactttgacaatcaagtaaaattttttttttttttttttgagacggagtcttgctctgtcccccaggctggagtgcggtggcgcgatctcggctcactgcaagctctgcctcctgggttcacgctgttctcctgcctaagcctcccgtaaatttttttattatagaaatggatggcttttcagattatatatacttggtttctatacactattttatttttgtaaagtagcagttcttttgctcaacacctgaattgcccccacaataaatttttagtttttcttcaatattcaagtaatacataacttttccttttcctgtttaacaaagaaaaaaatatataaagcaagctgttggacctccattgggtgttgtttaccaccactgtaggtgatcgtggcattgtccacctcagtcttctcatggctgtggattcaagttaagaaattcctgaaggtagcattccaggtagtctgtagaacagcccaaactctctgaattagtattatctctgataggtgttttttttttctttgcttttttatttgagacggggttttgctctgtcacccagcctggatttcagtggcacaatcttggcttactgcaacctccacctcctgggctgaaacaatcctcccacatcagtctcctgagtagctgggaccacaggcacatgccaccatgcccagctaattttttatatttttgttagagacagagtttcactatgttgcccaggttggtctcaagattcctgagctcaagcgatgttcccatcttggcctcccaaagtgctgggattacaggcatgagccgctgtgcctggccctggtagttttcttcttatatctttctcctgttgatctcacttgattttcttactagtctacaggaaatttcacgtggagattctgtgcctgtcctctttctggaatatcttccttattttagcatctcaacatgctgttcagcttctggcttgaaaccacccctctcccatcccaaccaagttaagcagcttctttcctgcttagaagtaccataacatgtgtattaatctttttttttttctctctccttcccttcattttaagctcctttttattaatctgtagggcagagcttagaaaaaggtttttgtatattgtaagtactaataaaatatttgtgaagttgatttgggacttgggagacctctcttctgtaagcaactcaataaataattatttaagtaccatcctcacccagtgtactgttagctgctgttatgatattgattactgtaaaattttaaagtatatccttggtagactgttgatcaaagtcagaaagcttgagaaactctgaggcaaaaaaaaaaaaaaaaaaaaaaaatgaagagcatgaaatgattctgcaaactgatcttacaatgatctttctttttttttttttttttttgagacagagtctcactctgtcacccaggctggcgtgcagtggcaggatcttggctcactgcagtctctgcctcccaggttccagtgattctcctgcttcagcctcccaggtagctgggattacaggcacacgccactacgcccggctagtttttgtatttttagtagagatggggtttcactatgttggccagactggtcttgaactcctgacctcaggtgatctgcctgccttggccttgcaaagtgctaggattacaggcatgagccaccatgcccggcctctttttttcttttttcttttcttttttgtacacagtttcactctgttgcccaggctggagtatagtggcgtgatcatagctcactacatcctcagactcctgggttcaggggatcttcccatctcagcctcccaagtagctaggactatagacgtgagacacgatgtctggctaattttttacattttttattttttgtagagttggggtcttatatgttgcccagggtggcctgccaccttgtcctcccaaagctgggattataggcttgaatcagatcccagtgatctttatccctcagctgcatttatatgggctaaattcatacatagtatatcaagatgtcttcaggaagtttaaaaaaaaaaaggtatcaaaaagacagtatgtcatgactgctttatctattttagtctcttagtttttgatactaaagactagaccggagaaatgctttaacaaatcacaaaaatctaagaaaatagacagttaattatttttaaacagtggaagaatttcaatcataattaaagcagcttttgtattttttgatgtcatagcctatagtgaaatttcatatattctgtttgtagatacatgagttcctacgatatgcttgcacataatagaactaattactatttgttgagaacctaacctgtacctaatatacatgtagtttctaaatcaaacattttactttttttaaaaattgtaaaaagcccatagtaaattaatcatgtttaccatttaaaaatgtacagttcagtagtgttaaatacattcacattgttgcacaacagatctctagaacttttttgtcttgcagaactgaaattctgtacccattgagcactaattcccacttccccctccccccaaattcttggcagccacctttctactttctgtttccatgatttttgactgttttagatacttacatgcatgggattaaacagtatttgcccctttgtgattagcttattttgcttagcataatgtcattgaggttcttccatgttgtagcatgtgacagaatttttttcttttctgtaaaggctgtatactatttcgttgtatgtatagactgcattttaaaatccatttatctattggtggacatttgggttgcttggtgatttggtgattcactattcatggttagccaggagtaatatgcagtaaatgttggtatacaaatatctctttgagatcctgctttgaattcttttggatatatggttgcccattgaacaacacagctttgaactatgtggatccatctataagtggatttttttcaaccagccatagatagaaaatatactattcggctgggcgtggtggctgatgcctgtaatcccagcacttgggaggctggggtgggaggatcagttgaggccaggagttcgagaccagcctggacaacatagtgagacctcatctctacaaaaaataaaaaagttagctaggtgtggtggcatgcgcctgtagtttcagctacttaggaggctgagacaggaggattgcttgagcctgggaggtcgaggctgcagtgagctgttattcatgccattgcactccagccagggcaacagaacaagaccctgctcaaaaaaaaaaaaaaagaaaatacactatttgtgggatgtgaaacttgtgtatagggtgggtgggctttttgtatatgcaggttccataggtcagatttgggggtttgagtatgagtgaatttggatgtatacaggtggtcctggaaccagtcttattcttcctcttcttttttttttttttttttttttaattattcgtcttttattttttggaaccactcttctgcatatgctgtgggatgactgtatatccagaattgtgattgctggatcatatggtagttctattttaatgttttgagtaacctcagtactgttttccataatggctgcacgatttttacagtcctacccacagtgcacaaaggtttcgatgtctctgcatccttgccaacacttgttattttctgtttcttttggtagtggccaacataatgggtgtgaggtgctatctcattgtggttttgatttgcattaacaatgttgagtatccttcatgtttattcaccccaaatattttacttttgaatttattgaaaatcatgcattttaattatgtaaaactcaataccatgcatcatttgttaaatgggaaaaggagaatttgtccttatgactattataaaatattctaataaggtgaaccttttttttttttttctttgagacaaggtctcgctgtgtcatccaggctggaatgcagtggcgcagtcttggcttactgcagcctctgcctcctgggttcaagtgattctcctgcctcagcctcctgagtagttggtattacaggtatgcaccaccacacctggctaatttttgcatttttatagagtcggggtttcaccatgttggcctggctggtaataagatgaactttatacttttttttttttttttgagacagagtcccactctgtcacctaggtgcagtggcacgatgatggctcactgcagccttgacctcctgggctcaagtgatcctccacctcagcctctcaggtatctgggattacaggtatgcaccaccacccctagctaatttttgtatttttttgtagagatggggtttcgccatgttgccccggctggtctagaactcctaggatcaagtgatctcccaccttggagtccccaggtgctgggattacaggcatgagccactgcattgggccagaactttatacttattaaacagtatgctgatgataggaaaaaactgtgcaatccttattcataaaattctgatctaaaacgtgttatattaaaattaattaataatgcaagcaaaattggcaaatttatttgtaaaccccattttgggctacttcgaaaattatttatggatcagaaaaggttgggaaatacttttctagagtaattgagcaggccctgtagatggcagaagtgttgcttgaactggcgtagaggaagaaagtgttgaattgccccacttaattccttaagtaattttgcctctttggagaatatttgcctgaaatagagaactcaagttatgctatgaagattctttttgaaaaatagagacagggtctcacattgcccaggctggtctcaaactcgtgggctcaagtgaccctttcaccttggcctcccagagtgcagggattacaggcatgagccatcatgccagaccaagaagatattttggagggctacatcccaccagtagcttgatgttgtacaaagagtggtaggctctgggcgaggctctgccattatactagttgtatttccttggcacgtagcttttgtggatctctcctcttcatggtgacatgaatgagatgaataaataatggaacaacttgtgtttactgagtatctaccatgtatgtgcctgcagagaggatgtcttcattttgcagatgaagtaattagtaaataacttgcctaggttccctcaggtagtgggcccttgaagaatgggcttttatggccaagtgcattggctcacgccagtaatcccagcactttgagcggctgaggtgggaggattgcttgagcccaggagttcaagaccagcctggacaacaaagtgaggccccatttctacaaaaaattagccacatgtggtggcatttgcctgtagtcccagctgcatgggagagtgaggtgagagggtcgcttgagcccagaaggtagagactgcagtgagccgtgtttgcacccctgcactccagcctgtgtgacagagtgagactgtgtctccaaaaaaaaaaaaaaaaaaaaaaaaggctattgctgcctatagggtcctttttagtgcaaatactaggattctaagcacttggtttctgttacccaggtcaaactatttctttggagagtttgattaccatcacaggtgatggtaagattctccctagctggcatttaatccagtggttatatgcatctttcccaaattctttatcaagttggtcaattcatagaacatctaatgctgattataagcctattatcttgcttgtgtctctgcactatgggcataatagcacctaaaagcttttgctataatggactatgatgtatctgaataaattacctttataaacaatcaaatctaaattggcaagttcaaatttacccaatttttcttatagaatttacctttgcagttaaagaaataattgttttttaccatttgtctagtatgacttgttatttacaaatagcatgtaactggccagctgtactatacacattttctagaaaagaaaatttcttcaaatttcttattgggtgccttagtcttttacccattagggctattcaaataaagaatgagatattctcttattctctagaaagagacattctatactaatggaaataccaacttttaaatctagactaccaaaaaaaaagtcatgtttttctaccctagaggctatagattcttaatgtcagccactcatcctggccctctcattttcttcttttcccatatggaagttctagggtgatatagtttggctctgtggccccacccaaatcacgcgtcaaattgtaatccccaaagttggaggaggggtctggtgggaagtgattgaatcatgggggcagacttttcccttgctgttctcctgacagtgagttctcgtgacagtgagttctcgtgagatctggttgtttaaaattatgtagcacctgccactttgctctctcttccccctgcatcagccacgtataatgtgctggcttcccctttgctttccactgtgatgataagtttcctgaggcttccccagccatgcttcctgtatagcctgctgaactgtaagcaaattaaacctcttttctttataaattacccaccctcagctctttatagcaatgtgagaatggactaatacagaaaatatgtaccacagaagtggggcatcacaataaagatacctgaaaatgtggatgcagcttttggactgagtaatgggcacaggttggtacagtttggagggctcagaacaagacaggaagataagggaatgtttggaacttcctagagacttgttgaatggttttgaccaaaatgctgatagtgatatggacagagatggctaggctgatgaagtctcagatgaagatgaggaacttattggggaactggagtaaaagtcactcttgctatgtgttagcaaagagactggcagcattgtgctcctgctctagggatctgttgaactttaaacttgagatgtttagggtatctggcggaagaaatttctaaaagcagcaacgtgttcaagaagtggcctggctgcttctaaaagcctgtgctcatttgcataagcaaataaatgacctgaaactggaacttaaatttaaaagggaagcagaacataaaagtttagaaaatttgcagcctgccatgtggtagaaaagaaaatctcactttctggggagaaattcaagctggctgcagaaatttacataagaggacgatacatggggtaaaatgctttttcctcccctaaaagccttggtattaatctggaaatagcaggaaggtaactgtaggaattatataatgggtttccctgtgcttggtcatgtctttgtatcatttccataggtcagtggagactcagaaaaccagtttctctgggcttttgataagtaatgttatagtaatcaacagatggtaactttcggtaaaacaaattgtcttcatctgttttgtgctggtataacggaatatctgagactgggtaatttttaaaattatttatttatttatttttgagatgggagttttgctctttttgcccaggctggactacaatggtgcgatctcggcccactgcaacctccgcctcctgggttcaagtgattctcctgcctcagcctcccgagtagctgggattacagttgcctaccaccatgcctagctaattttgtatttttagtagagacagggtttcaccatgttggccaggctggtgttgaactcctgccctcaggttatctgcctgcctcggcttcccaaagtgctgggattacaggcgtgagccaccacgcctggccaagactgggtaatttataaagaatagaagttggccaggcacagtggctcacgtctgtaatcccagcactttgggaggctgaggcgggtggatcacttgaggttaggagttcgagaccagcctggccaacatggtgaaaccccatctctaccaaaaaataaaaattagccgggtgcgttgatgtgcacctgtagtctcagctacttggaaggctgaggtgggagaatcacttgaacccaggaggtggaggttgcagtgagacagaattacgccactgccctctagcccaggcaacagagtgagaccctgtctcacaaaaggaataaatatatatatatatgcatatatgtatatatatatgcatatacgtacatatatatgcatatacgtatatacgtatatgtatatgtatatatacatatatgcatatatattatatataatatatagtgtatacaatatatgtatatacgtatatatatgtatatatgtatatatatgtatatacgtatatatatgtatatacacatatatatgtatatacgtatatatatttttcacagttttgggggctgggaagtttaagatcaaggtacctgcatctcatgaaggctttcttactgtgtcctcttatagcagaagatagaaggggcaagctgggaataactccctcagtcagactcttgtataagggcattcacaaggcaggaaccgtcgtggtctaatcacctctgaaaggccccatctcttaatactgttacatggacaacacctgagtttttgagggacaccttcagaccatagcacagatactgacaagtctcatcaattattaagtaaatagtacttgaagggtttgaaattttgtagtaccccggcagtgtgtttatatttgtgataaattttaagtataggaaaatcatggaaagacttactgctcataactgccgtacctcgtttacctctatctttccctaaactactactgttcttagcaagatcatgaactgtggggccgacataagccagaggagtttctgaagagtctgcttcagtgaggggttctgttgatagccatagaataaagaaatggcactttgcagacattggtacttccttgaaagaactggctgtggcatatacagaactcaaaaattgaaattactagataatggttacttggtttagattgcctggaaattatgagaggttctgtaaatgctttgggattagtagtttgaaccacctagtccattttatgattctgtcttcagaatatagaaggaattgcatcttatttggggattaggatataaagttagtgtacaaggcaaaatagcatgtaggcaaagttacccatgcagagtttgtgggaagatgccttaccacaaaccagtaccttctttttaggttcaagaccagcccatttttcctctagaatgagaacagatcattgctcttctgttgagcacagagttgttggtttgtacttaagggcaatcctgttagaatcactcctgagtggctaaatatcttcagtctgagagacttgtccagcaggttatgggaacagcagattcatgtcacctgtctcttgctcacttaggcaaacgcttagtgtctggaatggtgggtagaacccttgtatttactaccaaaattgagctttctctgacatttttgctcaatgtatgttagatttgtgtaagttaaattagatgttggcttatgcgtggtataagatttaaaatagacttcttagcaaaaagaacacctggtatcagattgacttaccccttcattttgtttgcttttattcagtgaagtgttgaaataacaggtatctcatgtctcatctttgaattataatgaattttgtatcatagctttagtttctggtacttttttttttttttttttgagacaagtctcgctctgtcacccaggctgaagagcagtggcgcgatctcagcttaacgcatcctctgcctcctaggttcaagcgattctcctgcctcagactcccaaatagctgagatcacaggcacctgccaccatacccggctaatttttgtatttttagtagagatggggttttaccgtgttggccaggctgatctccaactcctgacctcaggtgattcgcccgccttggcctcccaaagtgctaggattacaggcgtgagccacttggccccagcctctgatacctatttttttccgtttttcctccaacagtgtttctgtaaactgagtatatactcgatgaaatgagaagatttaacttataaaatcgatttagtatagttttcaagaaaaacttaaactttttttttctttttttccttttttttttttttttgagacagtctcactgtgattgcccaggctggagtgcagtggcacaatcttggctcactgcagccttggcctcctgggctcaggtgattggttctcccacctcagcctccagagtatcttggactataggcacatgccatcacgcctggctaatttgtatttttagtagagacagggtttcaccatattgtccaggctggtctcaaactcctggactcaggcaatctgcctgccttagcctcccaaagtgctgggattacaggtgtgagccatggcccccagctgaaaactttaacctttagatgtcaattcagttagtgagtctgttttatctgttgttcaagcaaattctttaaaacacttaaaatagttcagttcttcagttggttgaaatttatatggccatttcttttctaagcagtactctcagataaacaccagaatgctggtctatggacttcagtttgaaatcaatagtttactgtcttgctggaaggaggaaagtgatcttactgttgtggaaaatatatcactgcagaatctactcacttttgctgcaacggtcaagttaatgttttctatcattcagctaagattgctaaattagtataactttcctttgcctttgaactttttagcactacagaaaccaggactacaaatatgttctttacttttttctgttattttcccctatcatgagacttcttgaaggcgaatgctgttcattatgctttctcatctgtactttcatgtacatgtaatccactgtagtgaaggcactaaaacctgttgtattttccacatttcgatagatctctcacaggatttaacacttttggaatgctcagtaaattattattgactgcttgataatacctctttgttcctgcctttccccaagtccttcaacagaagtttcttctagcactttgcaggtcagttgagtggactatttgagtccctcacagtaatgagcttgtgtgtttttgtcattcttttttctttttttgaaaaaacaatagagtcatttaaggaagcaaaaaggtccctgggtcaagatttttattttatcttttatggtggtagggtgaggtgtgagtatttagggagatggttggtaggatggcgttgtcttagggttgcaggaggatcagaagggtgactgtaactactgatgatatcttacagtggtgaaggagaagataagtgaaaacagaggcaatttgtttaaacgaattttagtagatattagtctcttaatttggcactgacatatgacagattatttcatttaaattctgtagtttgacaggtataataaagtacccttcattaaatattcttcacatatactcttgtttgcctttttctagttgcaagagtggtgatttctatgtttcattataaattttagaaatatataatttgaatatattgtctgttctaggttctctgtgagtcaagaaaaggcagggtattatgacttcggattcgtatgatgtctggatatatttaaggggagcagttctcaaagtgtggcctgggattatgggatcaaaactattttttctttctttctttctttttttttttttttgagacagggtctcactctgtcatccaggctggagtgcagtggcgacatcttggcccactgcaacctctgcctcccaggttcaagtgattcttgtgtctcaacctcccagaaactattttcataataatactgagttgttccttgcttttttttaaaaaaacaaaacaaaacaaaaacaaacaaaaaaaccatgttggacatttgtacccacggtgcaaaagcaatgaataaacactgttggcaccttagcacttgcagttaaaaaagccagttttgtttgagaatgtccttgatgaagtactaaaaatgattaattttattaaatcttgatccttgcgtgcatgcagttgtaacattctgtgcaacaagatgggaatgtgtgtaaagtgcttctgctgcatattgaggtatgttgggttttgagcaaaagcacttacgtgattaagttgtgagctccactagccacttttttcatggaaatttcatttttacttaaaagagggtctgacaaaccatgattattcagacttaggtatttggcagactttttttaaatcaatgaagtgagactgtcacttcaaggaaaacaactgaccatatttgttggcagagagaacatttgggctttcaagcaaaaattaaaatttttggaaaacttttgtctgttactgtgagcatgacagcttcccagtacttaaagactttctgatgagattaatgggtttaacaacatgattttttgaaatatgccaacttctggaaaatctgcgtaactcagtggaccagtactttttagatgaggaatgatgatttataacattgtgcctgagttaaagattcatttaaagtgaatagactaatgaatgttaatgtggcagagtaggaaaagttcactgatacaatttcggattccatgttgcatctaatcttgtaagaaagaaattaccactgctaactgaagtatggtggttattttgaggaaaagcacctgtgagattgagttgtgaactgaactagccactttttaatagaactgttttggtgtaatatcaaagcacgagatccacaatgacctcagaaggctattcaaacactctcccttttctaagctaagtatgtttgagactggattttcttcatatacttgaaccaaaacaacatattggaacagattgaatgcagaaacagttacgagaatccgtttcctattagacagatattaaagaaattggcaaaaatgtaaagcaccacttactaattaattttttttttttttttttaaataaaagaaggctatgttgctcaggctggtctgaaactcctgggctcaagtggtcctcctgcctcagccccgcaaagtgctaggattataggcatgagcaaccacgcttgccctcactcactaattttttttaaaggaaaataagtttctttttaaaaatgtgttatttatattaggtacagttggccctccatattttcaggttcagcttctgtgcattcaacaactgcagatcaaaaatacttgtggaaaaaagccaataaaataatagaacagggcgggcgctgtggctcatgcctgtaatcccagcactttgggaggccaatccgggtggatcacctgaggtcaggagttcgagaccagcctggccaacatggtgaaaccctgtctctactaaaaatacaaaaattagctgggcatggtagtgtgtgcctgtaatcccagctactcaggaggctgagacaggagaatcgcttgaaccctggaggcggaggttgcagtgagccaagattgcgccactgcactccagcctgggtgacagagtgagactctgtctccaaaaaaaaaaaaaaagatacaaataatacagtataacaaggatttatataacattttacattgtattagatgatcataagtaatctagagccagatgatttaaagtataaaggagaatgtgcataggttatatgcaaatactgtgccattttatataagagatgagcatctgtggattttggtatccatgaagttcctggaaccaataccccctagacccccatggatactgagggcttgctataacagtttttaataaaaataaaaaacaggccaggtgtagtggctcacacctgtaatcccagcactttgggaggccaaggtgaggtgattgtttgagcccaggaattcaagaccagcctgggcaacatggcgaaaccctgtctctgctaaaaatagaaaacaatagccagacatggtagtgtgcacatgtggtcccagctacctgggaagttgagatgagaggatcacctaagcccaggagggcgaggctgtagtgagccgtgattgcgccagtgcacttcagccagggccagagtgagaccttgtctcaaaaaaataaaataaaataaaagtaaattgtaaaaaactctttagtttttcatttctattatgttaaatgttaatagctataacacatataaactaaacaggtcatgagaccaaaatgctgaagaatggtgttaatggtgttcttacttactttgaattaccgtttttatttttctatgctagctaattactatgtgaataattaacctcaatgaagttctgtattctctgttgtagcagtggctgatttagggctcagttgtcttttgacctgttcttccttctcatatgattgcatctcgatttttaattttagacttagtttttaattctaaaattaaaaatctttttttttttttcttctatgcccactgctgtcaacccagtcagggcccttatcactttattcttgaattacttcagttctccattttttttctcatgtggtgtttttcaaacttgagtgttgattggaatcccagggttaattcaactttaattttatgaatatgatttcatataagttccttatgataaaaccagagcagatttacaaattaaatatagaccatcaaattagctatacatatttaaggatgagattttgttaaaaccaaatagtcaatgttagagcttatagaatggcatagcctcaggatcagctttataatcagtttttttaaatttggcttgataatactaacagagaatggctatttgcataaatatgttttataaaatggtgttaacttttagtctttgttcaggataatgagacctactagttaaccagaacggggccagcaagcaactcttgaatgctagttttaatgcctgatcaaaattctgtaaagctgtgttgatcactgaggcgttgcggatatactgagcctgcattaagtggatatttaatctaattatttctggaatctttaccagaaaacatgctcttacatagtttgctcctgggtttgctgttggtgaactgccgttggttggtgggatctctgtgtcccatgggctttgccacacagctggggctctgagtacttcagctcacattcctttgtttgccacccttgccttaactcacttacctgtattgttttttctcccatagcactcaacatcatcttacatgtaaacattttactttgttgttgttgttgttgttgttgttgttgtttttgagacagagtcttgctctgttgcccaggctggtgcagtggcacatcttggctcactgaaatccctgcttcctgggctcaagtgattctcatgcctcagcctccttagtagccgggactacaggtgtgtgccaccactcctggctaatttttgtatttttagtagagacggggtttcaccgtcttggccaggctggtcttgaactcctggcctcaggtcatccacctgcttcagcctcccaaagtgctgggatcacaggcatgagctactgcacctgacactttgtttttaactgtttctgtccaactagattttaagctgtataagggcacagatttttaattgttaattgctaagtcttcagtttctattgtagtctctggcttgtattagatgctcaataaatatttgttgacttaatgaatgcatattaacccttcttttctttattcaactgcaaactcaaatactcttttctgagttgtgtgctctcactgtgggtaaagaccagtttttctcttcattttaaacacaactaaactggccctgaattgtatagaatccttttgatttgttgtttgatcagtacaagttcagtcagtggcacagttacttcccttgattcagattctgtagaagcctaaaagtgcattatctcttcaagtagacatgtcatcatataggctgctatggagatctttttttttccttttaaattgtttgtgtaacagtttctgttttaaaacagtggaattaccagcctggacactatagtgagacactttctctacaaaaaataaattaaaaaaaattagctgggtgtggtggagtacacctgtagtccccagctactcgagaggctgaggcagaaagattgcctgagcccaggaatttgaggcttcagtgagttgggattgcatcactgcactccagcctggacgacagagtgagaccttgtcccttccaaaaaaattgaaaaagtagaattatatttgggagtttttttcctcaaattcctttgaagttaagatttataaaagtatgggaggccaagctgggattatcatttgggcccagatgttccaggctgcagtgagctacggtcgggccacggaactccagcctggggaacagagtgagccctgcctcaaaataataataaaaataataataatagtacaagctctcttaattactacctgttttttttttgagacagagtcttgctctgtcacccaggctggagtgcagtggcgctatctaggctcattgcaagctctgcctcccgggttcacgccattctcctgcctcagcctcccgagtagctgggactacaggtgcccgccaccacgcctggctaattttttgtatttttagtagagatggggtttcaccttgttagcaagaatggtctcgatctcctgacctcgtgatctgcctgccttggcctcccaaagtgctgggattataggcgtgagccactgcgcctggccactacctgtttttttttttaatcctatgtttttaaataattctaatcatctattcatattttttgtttctcctatttaacattttatgtaacatttttagattttaaaatttgtttctgttatcacttctaaaagtgaatgttatttttaaaaatgaatgcctttacattgggttgttatgtaacctaatttgctttaacaaataactttattttggagtaattttatttttaattatttatttatttatttatttatttgggacggagtctcgctctgtcgcccaggctggaatgcagaggtgcgatctctgctcaccgcagcctccacctcctgggttcaagcaattctcctgccttagcctcccaagtagctgggattacaggtgctcgccatcatgcccagctaatttttgtatttttagtagagactgggtttcaccatattggccaggctgctcttgaactcctgacttacgtgatccgcctgcctcagcctcccaaagtgctgggattaacaagcatgagccactgcacctggcctattttggaatacttttggatttatatagccatggtatatttgttaaaactaagaaatcagtatcagtatgttgtcattaactaaactcaagactttatttggattttgccagttattccactaatgtctttctttggttggttggttggttggtttcaggatccaatccaagataccacagttcatttagttatcatgtctcctaagtttttctgatctgtggtggttctcagcatttccttatttttcatgacctcttaatcgttttaaagtgtattggtcagatgttttgtaagaatgtcctttgatttcggcttatcggatgttttcttatgattaggctgaagttatgggttgaggtctgggggaaggataccacagaggtgaaatccccatctcatttcatcacatcggggagtacataatgacagcagtacttaccattgctcatgttaatggttatggttttttttcacagaaacattactgtttccctttccagactcttctttggaagtgaatcattgaatctagctcacgctcagggggaaggaaattgaattctccctcccagaatggggacatacactcatgtatcagttggaattcttgtcaggagagttgttccttctcctccagttgtttatttaatcacttatttatatcatagactcatgaataattattttattctgttataatactgttatgatttactctgctgctcaaattattccagctttggccactgagagtttcttctgtttgcttccagtctcggacctatccgtctcctttcatctttggagcatttcccttcttctactggcttgccttgtagtttccctgtcccaaccctagaatcagctgtttctgcagaccctgcttccttgtatttattggaaggtggtatttagaaacaaaagtcttgggtgcagggtatgcttattttattgctcctggggtgtcactgcttccaggccctctgttggacagaccctggaaatgcaggtatttcagctaacctgcatgtgcatacatacctacgtttctgtatctatccgcatatattttaaaatacacgtgagttcatattgatatatctgactaattcatcaccacctcttgcttatttgtaacttctttctgtgacattgagaaacttggcacacattatctacattttatttacttatttgtttaaccctagtgtacatgtagagtattttcagaattacagactggtacccttatgagaaacaaatttaccagctagagtacagagtagattgtccaaagttctgtagattagctccccccgacctcctgctccttccatgagattatctcataaaattataatactgttagacttgctgtatagtttgcattctatcctgagatcccctgacatcctgttttgtttttatttgcatacagtttaccatttaaaaaaagtctttatattgagttgttaactttttaatataaatgttgtaattcctgatattactcatctgccatttatgtagttgatagtcactacttttttttttttttttttttttttgagatggagtcttgctctgtcgcctaggctggagtgcagtgacgtgttctcagctcactgcaacctccgcctcctaggttcaagcgattctcctgcctcagcctcctgagtagctgggattacaggcgtgcatcaccacgcccggataatttttgtatttttagtagagacggggtttcaccatgttgggcaggctggtcttgaactcttgacctcatgatccacctgcctcggtctcccaaagtgctgggattacaggcgggagccacagcgcccacctgatagccactacttttttaatgctgatatagtgcttaccatctgtcaggcactgtctatgcaccgtaaataaatttacttctcacaatgactctgaggtggaggctcttgaattagccttttttcatgaggagagtgaggcacaaagatgtgcagtaacttggccaacttcacacccagctgctaagtgggagaacttggatctgaacccagggaatgagctttggagtccaaactcttgctttttgtttaatgctgtgtataggacttagaattaatacaactaactcttctcttattagtttagtttattacttggaatttggtgtgaaatagagagtgctgctaccccaattctttgccccatgtgtgtaacaggcattattaatcatgacagcactctctctgctgagctagaacatggcctggcctaaggccaataatccttatggacacaaccctcaaagtggttgtatgagatgaactctccttgccatctctgatcttgtctgttttgaatcctgattctagtgcccagtgcatggcactgcgtgcctgcttctcctggattcctctctcatggtcttcctttttacagggacaggttttctatattccccctcaggcataggatctgtttctcttaagtaattaatttttatcacttaccaataatttaacatctatatgtctaagttctctttatattgtttttcttatccttacctacaaaatctcatttaccattgatttaaatttcagtatattgaaacaatggattgtaacattaaaaataaatgaaattgccaaatgatactaacagctgccttcaaagcatttaaacatacaaacaggcactattgaagactggagagttatcaaaattggcaattttttggcagtcttgctgtgaagtaaacatttttaaaaggaaggacattgagaatattgtaatcatattacttacatcttgttgggtttggatttgaccttacatagcctggtattcagttaagagtcaaagttgctgctattaatttagttgagaaggataaatagtacatgtagtttattgttttctttagagaaatagaaacagaaacatttatttggccagaccaaataggtggtcattcccttgatttagcaaatccttgaggtagaattttttttttttttttttgagacggagtctcactgtgtcgcccaggctggaatgcagtggcacgatctcggctcactgcaagctctgcctcccgggttcacgccattctcctgcctcagcatcccgagtagctgggactacaggtgcccgccaccacgcctggctaattttttgtatttttagtagagacggggtttcactgtgttagtctcgatctcctgacctcatgatctgcccatcttggcctcccaaagtgctgggattattggcgtgagccaccgcgcctcccggtagaatttttaaagaagcaataagggctctgacaatacctatgggacttttttcaaattattcagttattggcagctgggtaaagagggacaggaatgtctgatttaggtaaatacctctcacaagtggagtaggggcgtatctggtagagaacagtgctgattccttggacttgtgtggcctggccacctgagaggtaggtaggaaggtgtgtcttggccatgtcctggggcctctgtaaacctgagaggatggaggaagctctactttttctgacgatgctgttggtgataatgaagtaggctatgagagcccatcttccattaattcagatgttcctatacagttggccctctgtaaatgggattctacatttgtagattcagccaactgtgggtaaaaatacttgaaaagaaacaaaagtgtgtgtactgaacatgtacatacttttttcttgccattattccctaaacataatagtttaacaactatttatgtaatatttacattgtattaggtatttagtaatctagagatggttttaaagcaggggtccctagtccccaggccgtggactggtacccgtctgtggcccgttaggaactgggctgtacagcaggaggtgagctccgcctcctgtcagattggcagcagcattagattctcacggaagcgtgaaccctattgtaagctgcacacgtgagggatctaggttgcacactccttaaaagaatcttttttttttggagacggagtctcgtctcactgtgttgcctaggctggagtgcagtggcatgatcctggctcacctcaacctctgcctcccaggttcaagtgattctcctgcctcagcttcctgagtagctgggattacaggcacctgcctccacacccagctaatttttgtatttttagtagaaacagggttttaccatgttggccagactggtctcgaattcctgacctcaagtgatctgcctgccttggcctcccaaagtgctgggatatgagccaccatgcctggccatccttatgagaatctaactaatgcctgatgatctgaggtggaacagtttcatcctgaaaccatgtaccctctcccctctttgtggaaaaactgtcttccacaaaaccagtccctggtgcaaaaaagttggagactgctggtttaaagtatatgggagaatgtgtatacgttatgtaatagtgtaccattttatatcagggacttgagtcttcgtggattttggtatctgttgggggtcctggaactaatcctccgtggatatggaggaacaactgtatatatggtctcagtaataaatacgtatctgagataccctctgtattaagaaaaaaaagatgggaatttcttggctcatataactgaatagttccgaaatttgggaaggtctgcctgacttagggcttaggtcatgagactacagttctttcttggctttgacttttgttttttgtagttgttgactctgttctttgaggctcttctccaagatggcttccaacaagtccttgggcaaacatttttccaggtttagacctaaggggagaagagtgagcattcttgtcctagcattgcaggaaacctgagattttcactgtgactgcactgattgtggccgtgtatctaaccctgtactggtcactatgtctagggagatgtaccttggtcaggccagggtcacaagcagcttgagtcagcttcctccagtgtacttggtagttctcagagaagagttggtgtgaagttcccaagagaagggtgaattgaccctgggtggcaaaaacaacatagcatctactctagtgggataaagtaagatggtaaggcatggtgttcagggtatttctgagtttggtttgattcacaaaatagattgtgaacactgtaattgcccttaattttagttaacctatgtaaatgtaaatctgtgaggtgtcactacattctgctaaagctttgatgggctcctgccatattctggtttggcttttgtattaaagagccattgttgttttggtttcactgtatacaggtagacttttgttttaatgaatagaggtataatctgttttgaacaggaaaagctgcttttgtattctggataaagttcatactagtgggatttcacctatgaaactaaacaaatctttgaaaaatttaaaaaccatatgtatttacacagtgtccattcagtagaagtcgtttaaataaaggcttctgccaatatatgaatatttagagctgtattttattattttactttttttgagacagggtctcgctctgtcacctaggctggagtgcagtggtgtgaagacagttcactgcagtctccacctcacaggctcaagagatcctcccacctcagcttcctgagtagttgggaccacaggtgtgcgccactacacctggcttgcttgcttgcttatttattgatttgagatgggagtctcactatattgcctaggctggtcttgaactcctgggctcaatcctccaaccttggcctcccaaaatgctgggtttacaggcttgaaccactgtacgtggccttgaatctgtgttttaatactatgcttacttggctgtggtgttgtgaaaagatcactgaaaatggagtcagaggcctgatttgagccagtcgtttgttgtgggggaaggaggtcaggggagctaacatctaaaggctcactatatgccaggcacagaaccaagtgtgtttgcatgtatatttcgtttttgttgccagactttgaggtaggttttatggataaggtctttaaggcaatatcagcttccttttaaaaaagaaattccggaaactgagttttaggctgaagatctctaactggtagtagggacaactgaaccacagggtcctaactgaccctgcgatttatctccttttgcggggggtttcttgataatagggtgcactttacctcattttttggctcaagcatggataggccacccttccttttcatacctatagctaagctttacaaatgatatgctgataagatacaagctactcgttattcatgtgggttaatagacctgtttgtttgcttgtttttaagtctatagccgccccacccccaatctacaatttcaccttctaaggttttagttactcattcaaactgcagtctgaaaatgttacgatattttgagagagagaagactctagctacgtaacttttgtaacaatatattgttataattgttcattttaccattagttattgctgtcagtctcttactgtgccttatttataaattaaacttcatgggtatgtacgtataggaaaaaacatggtatatttagagtttagtactatctgcagctttaggcatccgctgtggggttggcggggggcggtcttgggagataagtggggactactgtacaattatcaggcacacacaggctctgggattttacaaatgagtaaaagtggttcttgctgttgaagcacttacagtgggaatagagtgaaatacatgaaaatgtgattttaatatgttataaatgctatgatggtgggagtttgttttgtgtaaaacatccttttaattggtactttaaattttaatattctttcacaggtctacctatttagtcttacactttcaaagaactacctggatgctgtagattttcatgatatactttattaggtatgttattaatggtagaaacagcatggaaagtcttccagaatattagacaaggacagttctagtactaaaacataaaatgctaactaatgtcttcatcaagacataaaatatgtatcttaaaaaataaattgtaagccaggcgcagtggctcacacctgtaatcccagcactttgggaggctgaggcgggtggatcacaaggtcaggagattgagaccatcctggctaacacggtgaaaccctgtctctactaaaaatacaaaaaattagcctggcgtggtggcgggcaccggtagtcccacctacttgggaggctgaggcaggagaatggcgtgaacccgggaggcagagcttgcagtgagcggagattgcaccaccgcactccagcttgggggacagagtgagactccatctcaaaaaaaaagaaattgtaataacacccacattatacatcagtgaaaactaaacacgttactaccctaggccttattgcacaggggtgctacctccaaggagaaatttgtctaggcagcagatggactagaggtgattagcctatgagcgaatgaggctacagatcattcctttttatctgattccttttctttctagttcctaggccttggaagcactaagtggtcttaagtaatttgcatagaattagttgagttcatctgttaactaactagcagataggaagaaaactattgtcatgaaattatttaaaaaataataatgctccagtttcttctcatctttgatgtcctttggtcctacctcactgccttcctaacaccattttctgctttacctcaaagctggggtcatcttgagtttagcctgcttaatccgagtgactgtcagctttattccactttagcaactcgcaggcaaggccacacttggaaacttttcacttggaatagttctatcttggtgatttcatcagccttctttatgtcaaatacactcaaattcctgcccattcttatctcttcgttcccttcaggtccttagtccttttaatttgtgactttcattctccaggtccattcttattaaatgtctgccagccagactttagttgccccctgtccagctttctcttgctcagacctaagatttctttaggttctttctttgccttttgaaatccagctcagcttttaagattgagttccttgttacctttcctgccatccttctgcagttcctaatattcttttctttctcccaaagtgcttttgtataaacagtcagccttccatatccgtgagttccaaatccatggatcctgaattcatggatttaaccagctgcagataaaaaatattcagaaaaaaaaagatggttgcatctgtactgaacatgtctgtactgttttgcttgtcattattttctaaacaatacagtataacaactatttacatggcatttacattgtattagggattataagtaatctagaggtgatttaaagtatatgggagtctcttatatcccaggaagccaggtaaaaaaaaaaagtatatgggaggctatgcataggtgatatgcaaatattacaccactttatatcacggacttttgagcatctgtggattttggtatccgaggggtgtcctggaaccagttccccagggatactgaagtatgtctgtctatctcatactatatttttccctttgtcttaggtagactatcagctccataggggcaaggatttaataatatttgtatattcattttattcaagttcgtatacactgcttgggtcataatatttattacatgcttgagaaaatgaatttcttcgcccctttgttacagctctgagtaaacagccatctgccttctctgtcatctgttggtggttgagtatttctgtagaaagttacccattggcctcaggactcttactctaaatcttcttcttaggcagttttctctgtgcatgaagtttttatgtaaacaaatagatgaagcctgccctactcatttatttgctcaagccagaaagtcaccttcttcttcactttccatatttaaatcatcatttggtggaattttggcctaagcaactcttgaattcacgtacttttccctgtcatcgccagtgtggtgtagaagcctctgtcaccccttgtcgggatgctgtgctgcagcatcatctaacctggttgcagttattctttcactccctcaccgcacacccttttacttaaaacactaaaagtggcttctcattgttcttaagataaagcacaaattgttagtgtggcctgtaaagctttgcatagcctgacagagaatgtcctgctaataatttgaaggtacaggatgattttaatactttaggagaaaatgttctaggaaaagacgcttgtttagacttaaggtgaggactctgcagtatgaattagacatctggtgaactataagctgtccccgcatttaaacataattggttctgagagcctgcaactaaagataaggcagaagaatttactttgcatttcctgcattcctcttttcgcttgatagcagaaacccctcatgttaataaaggtggcacaagaggcaaaaatacagactttatcacagtgtttaaggagaggtgcatgattaagtgtgtggggagagagtacctttgtacattttattatatggtgaactgtatgttttctacttttagtactgtttgtaaattttacttcttcttggatttacctttttcagttatattattccattatgccttgctactgtaacagctaatgatgaaaaacaggatctgtctttatattttcttccctccacaaatgtggatctcatagagttgaaaactaggttgtgatatagtatagtatacctaattcctgtaatgggatcatgttcctataatatggccgcaatttagtgtagaatttttgtaaataaaagtgtattttaagtttaacttaaactttcaatgaagtgttttaaggatttaaccatgcagcacaaatgagcacctttctgtaaatgccaacagtgtaatatgtgtcatttcttcactgattgttagtttgctgcggattaaaacacaggtgatcatattcaggctggttagattagtgattttaatatgaaaccattgcttttagaataatcatgggccagactgggaagaaatctgagaagggaccagtttgttggcggaagcgtgtaaaatcagagtacatgcgactgagacagctcaagaggttcagacgagctgatgaagtaaaggtataattttatcttttgtgaaaatgaatatacaattaaggataggttttttcccctcctaagttcaataagtttttatttaaaataattctagccaagtttgtatttacaataagatgagtgaatacttggaagcttaggttcttgatctgtcttaacattataccatgcacattaatcagtctttgtttagtatacttttatttgcatatgcagctgcagaaatgcctccctaaatataaagggatgcatttcatgattttatgtttctaaagtcattgtgattaaaaaaaaaaatacacacacacacacacacacacacacacacacacacacagtcaacaaagaaaggatggcaatcgtttcctgttctctaaccacttgcaataaaataacagatgtggtgtttttgtattatttgaatgtgggaaactatagtgtaaaatctatatgcacctaagaaaatgttcttgatatttttgaggatacactagatttttaacctgctttacaggtgttatacattttctaacaatgaacaatttctcctttcctctccttcattttttttttaagagtatgtttagttccaatcgtcagaaaattttggaaagaacggaaatcttaaaccaagaatggaaacagcgaaggatacagcctgtgcacatcctgacttctgtgagctcattgcgcgggactagggaggttggttaacataacatgttgtagtaattatcatcatcttttggtttatgctattggaacatttgtttggttattgggaggaaatcattgacctcagggtgtccatctttttattgctatttctttttaatgtgcaactgttaatgactgctgtagatcacactattagctattcctataacagagttctaagtgtttggaatatactttgaactaggtagatgcctagattttataagaatgttgatttaaaaaaaaaagcttttaaatcaaatactctttaatgtcctcatcagatcactatttagagcaaggttttctgactcactgttgtaaccccaatttgaattagccatgtggtaagaaatgttttaatattcatggacaagaaagctttttattatatattttgttcaaaggctaagcctctctataagaatttttaaatttcttgttgagagattggaatttctttttctatagtactttctgaacgagctagctcttaattactccaagagtttcctattttcaaagaaaggcagtaaatctatggtagaatctataattaaatatcccgttaaaacatgattgcagcaagatgagaagaaaatggcaaaagtctaaatagcttaggcatttctgtttttaagtgcctgtgttgtatagaaagacatggcataggatatttaaaggggagttgaagcaaaaggggaaagagcgagttggaattaaaattgcactatgtagtagattgtttatctgaatgtctaaatatgggaagcacctaaacaatggaatttgttttaagataatttttcaacgagtagccatttttaatataacattattgtaacaagcaacaatttgctattgtatgaggccttctggagattgtgtgacatatgagcttcaaagacttgtcagtgatatcccagtagcaacagacataatcctaatgcagattttagtgatattctggtggtcttgatggtgacacttggaatgattcttagtcatttatgcgttttgttaggtgacacctctgtaaagctctgacttataattgtataaatattgattatacagtgacactttcctcctcatctggtacaggccagagcctagatgttagaggtcagtggcatgggggaaagggattctttttgatgcccatcagaacgttcttaaagggaataaggatggggcttttaagtaattgctttgaaagtttttatttggtgaccttttagaaatgttgaattgaaagaaaaacctcattttctctctggaggaataaatcttagaaatttggagaaattaggtagattcacatggaaaacttatttttgccacccattatgaatctcagaatgctatcaaaagcctaacttaggtttataataaaagtcaacagcaataatgaattgtgtcactgtgctaggtactttgtacacattacctcttttaatcttcatgataaggaaggagtattacacttttttttttttaatccaggcaaggaaactgagccttagaagcatttagtcagtttgcctgctctaatgatctgatgtgggcccagaagagcacactttgctcagacacatgaagtattagtgaagtacattattgacatacaacttgaattgctcatagtatatttggcattaaatttaagagtattgtttgttcaagtggatatttactaattgaaattttcttttaattttgtcaaatggagacaaatcgttgaatataaagatacctaatgttcgtgagtttatgtgaaaaattgggagaagaaatcagcattttaataaaacaaaattgttttctaaagttagtacttccacagtaccacacacacacgcagattgagaataaacattttcacagagtatgggggaaaaggaggaaaataatccccagccttaggaatccagtttttgttgcatagaactctgacctgattaaggtcagtcattttttttgttgctggtgttggtagctgatcctgcttcagatgatccatctattatttcccctttccgcctttttaaaccatcaccactgacttttagaagccagagtttatatcgtttgtgataagcttcttggtgttatcacaataaatcgtaaagctttagggctaaaaaggactgtagtggtctcatggtccaacctcctgacttttcagatgagaatatatccctgtttattttttctttcgtttaagcatttccatctacatattatttcttttatatatttgaaagattctagtctgaagtaggtcaggtagggaggagaaaataaaagtgattcttgtttggcaaagactttcctagtggtaggcttggcagaatttcagactgcagtattgattcaaacacgtaaagtctatagcattcaacatttgaagtgccattatagcattgttttaattaactgcttgcttttataagctaattatatgtaagtgctggtgtcttcaatttaaatcctctgcagcatgacaaatcaatgacaatttttagttcttttgtcaggtcttaaacttatactcccatttggccgggcgcggtggctcacgcctgtaatcccagcactttgggaggccgaggcgggcggatcacgaggtcaggagatcgagaccatcctggctaacaaggtgaaaccccgtctctactaaaaatacaaaaaattagccgggcgtggtagcgggcgcctgtagtcccagctactggggaggctgaggcaggagaatggcgtgaacctgggaggcggagcttgcagtgagccgagatcgcgccactgcactccagcctgggcgacagagcgagacgctgtctcaaaaaaaaaaaaaaaaaaaaaaaaaaaaaacttatactcccatttgtagttgtagaagtggatatactcagtttgaaaatgactgattttctccaagttattttggtaaagaatgtcggacaaaagtttactgaagtgagtgctttagatgctgacttcaggtcagggagctctgcatttgaacccaaagaccacatctccagtatgtggttttcttactgtgtgtggttttgttttgtttttgtgacagggtcttgctctgttgcctaggctggagtgcagtggtacaatcacagcagccttgacctcccaggctcaggcgatcctctcacctcagcctcctgagtagctaggactacagtcacacgccaccacatcgggctaattttttttttttttttttttgagacggagtttcgttcttgttgcccaggctggagtgcaatggtgcagtctcggctcactgcaacctctgcctcccaggttcaagcgattatcctgcctcagcctcctgagtagctgggattacaggcatatgccaccacgcccggctagtttttgtatttttagtagagatggggtttcatcattttggtcaggctggtctcgaactcctgacctcaggtgatccacccaccttggcctcccaaagtgctgggattacaagtgtgagccaccgagcacatcgggctaatttttttgtattattttgtagagacagggtcataccatgttttccaggctggtctcgaactcctgggctcaagcaatccatctgccttggcctcccaaagtactgggattacaggtgtgagtcaccatacccgtcccttaatgtgtttttaagatactttagggcaactcttttttttttgtttttgagattatagaacctaagcattattgttcatagaattttaatacagaatgtgtttacttttaaaaaacacttctgactttttgaagacagtgactatatctgttttacgtttatttcttttattcttaagttaatcttttaaagaagcatccagcttcacactgtcattaatcaatacatataggtcaagggatgtacattttattcccataatatgtcactgttagtcaagaaattgttactaggtgactttcataactttgtcttgtcaaccagtaataactatgttctgtaggtcttcatattggtagttatattcttcattgtgtctctttttccatttttaatgaagaaactggtttaagagttactcttgaggttctcattaatgtatttctttttcgtacatctgcccatttcttgctgataatcacaagacacttctccttcactgacttcatatcaaaatgttttctactgtatgtgttatgactgaaagagaagtttgcttagatagtgcctgtcttctgggcaaatggtatgattagtttagtaatttaatccatcttgccaatatacatttctagattcccaggagaatataatgttcttgtaagagttttgtatcttgtttactaatgaaaagagagaatatggtgtatattcttggggcttcagaatgtctaatagaagcagcatcagaaggggatcacggtcctcgtatgaagaatttaaaaacttttggacttttaaatatttacagatttgctcttcatagtaatatttgaaatgattttaagttcattgcttagcatttgatattagcagatttttttggggggagaagcttctatcaaaataatgtgtaaattattttgtatgttggcagtttggacttaacatttttgttgaaggagaagaaaaatacctgttacatgtaaacactcgattgcattttttattgatttagtaatgaagttccttcagttaattgtaaatgaaaatttagtcactgtttacaaagaaaaaataatatcctagtgtatttcatgtataatttagtgtactttactcagatacattctttgagtgtagtaacatggtttctttatacattgcaggattataggccctataggtttagattctcatgaaccttttgagcaaatatggcagaaagctggtcacttttctcatgtctttgaattaagtggatggctcttcaagtgtctgtcttactcttagtctgaacatatacactcattcttattgcctcaaggaagtggtctctgagttgctttctgtagctccttgcctcttttaactattattggtatttacttggagagttgagtcttgttaggttaactgtcagtcattcccacctgtataacttgtaaatccatgcagcctttgctttctttctggctttatttaggctgaaatagatgtaacatttcttaaatgatcatataaggattttttttttcttgtgagcagaaactaatataagcattttatgcttttccaaagggttagcttaaaaaaaaaaggtcatttgtgatatttgagttcatatagcatcatatattaaatacatgtcatttattatgtttatattttaaatttgattatatatcttcagtttaaaaggtgaattttaaaaagtcaaatttaggcaactaataaaactagcagaagttgcttttcatttgtagtttttctcctagaatattgtatatatagtttgtgttttataaattattgttataaagatcatgtatcttccccaaatgcaaaaccgagttctgacaactatttaaaaataaaacaacaaatccaaacaaacaaaagtgcttggttaatctggaatgttagaaattgttttattctctgaagaaacaagaaaattgcattagaccgaggacttttgccataatttagctttataatagggattgacttaaatttactccttaggattctgtcttaccttctcttagcagattttataaaagaatatcacttgagtaacattttggaaaggctgggcagatttcataagctacccaacaaggcagttttatactagctaatgtgactacctgttgggctaatgaaactagtttaaaagttttatctttttctctccactgaacagatcattgtaaaatgattattgtaaaaatttagataacacagaaataggtaaaacagaaagcaaaagtccctgtagtccaactgctctggcttttattctttcagatttcattttctatatatactaggaggttaataatattttatttttaaaaattattggccagatatacatcctttccattttgggccatcactttacaacctctgtccaaattttcattcttttaaaaaatatgtagtgtttgacattatggatataccataatttattgccccattcttgatggacatttagatttttaatttttacttttgcaatcagtgctgtactgaactttatatacatccttgcatcttttgagtaattctgtagaagttcctacaaatagaattattagatcaagatgatatgcatgtttaaaaatttagcagatattaccaaatgcaccctcaaaagttttacattacacccccaccagctgtttatggaatgtctgtttacctatattctcaacacaatattatcaatcttaatcttcagtccaataggtaaaacattgttagaattggttataccgtgtcttaattattcagtgaagttaaatgtcagttactgtttgtttatatttgtgaattggctgttacagtgttggatcttgtgcaattaggttgggcttttcttatgattcataaggactttttgtatattacaaatactagttttggctgggtgcagtggctcacgcctgtaatcccagcactttgggaggccaaggtgggcagaccttttgagatcaggaattctagaccaccctgaccaacatggtgaaagcccatctttactaaaaatacaaaaaaattagctgggcatggtggtgtgcgcctgtagtcccagctacttgggaggctgaggcaggagaatgccttgaacccaggaggcagaggtttcagtgagccgagatcacaagactcaatctcaaaaaaacaaaaaacaaaaaaaaatagttttttttgtcatatgttttgcagattgccccccccccattttgatgttatttctgatatatcttggttatacagttttttcttttaatttttgtctttcttaaaggttccaaaagtctttcttcacttcaagattataaattatctacctatctttcctttagttctctgttttcatttttatattctaatatcatttaacccatcaggaatttcctttggtgtaaagaatgggtaggggatttggtttttaattttgttctttttagtttcctggttgtttcaacactatttactgagtaattaatgaatagtgaggggcagttgtaaacttgtttttgaagatcagcagaatcagccttcttggttggtggtttcctgggcagttctaccactcgagcaagacctgaagtgtttcccttcagaaaccacagcctgtgccatcttgctcactcactttccttccttccttccttccttccttccttccttccttccttccttccttccttccttccttatttgacagagttttgctctattacccatgctggagtgcagtggcgtcatcttgtctcacctcaacctctgcctcctgggttcaagctattcttgtgccttggcctccagagcagctgggactacaggcatgcaccaccacacctggctaatttttgtattttagtaaagacagggtttcaccatgttggctaggctggtcttgaactcctgacctcaagtgatccgcccaccttggcctcccaaagtgctgggattacaggcctgagccactgcgcctggtcttgcccactcacttgaacctctactactttgtagtattaaacccagatccactgatttaagcaaggcttcaaaacaattttactgccacagcctatataaactatagatttatataatactctctgttccattttagttgatttttcccccaatgacagaaattgttcttttttctactcacaggactgttattgaattagaaatttgaatttacatgtggtggatgaacatcactcttgttacctgcttcatgtacttttttgagtgcttactacctgtgatgattattctgtttctgatctccagtacttagcaaaactggagtttttccgcaaagctgcagatgagggaatgatttaccttaacagcccttcaggattttaacctaggattaagtaggtttgcatgtttgtgattcctttcattcagggcccggcttctgaaggtaggatgcggggaagagctttaaggatctaaatttcctcccagcctgagcaacatagtgagatcccatctcaataaaaaataaaaaaattagccagatgtggtgacacgtacttgtagtcccagctactcgggaggctgaggtgagataatcatttgagcccaggagtttgaggctgcagtgagccatgattgcaccactgcactctaaccttggcaacaagagctagaccctgtctcaaaaaaaaaaaaaaaaaaaaaaggagtctaaacttcccgatactgtattccaaatgtcctatgttaaatgctgtgttatcattccccgccccttggaatgtagaaaccgttaattacaggataaaagtgaactgagaagcaaatacccttgtgttgtagggtagaggttcttcacccaatgtggaattggtctgtaacaaatactgaacaacacaaactgaaataatttacaatctgatatgaattactctttttcatttcttaactccaacacttgattagttgctttcattaaatacatctgtgtcaatatcttaatggggttttttcttccgtttttgtttctgcctctctcctgttggagcgactcatttcttttcaagtgtcgtcttcaatctcccttctcctattttgccttctcaacctgattatgaattcacttccattattgagcttctctttactttatccaaatttgctatttatctcattgcagtttttcttcagtacttactttgcataaaagtagcctgaacttaaattatatacacggtcattttcaccagaagggctagctaatttaaaacttgggcgtgttatcttttctttcctcaagtcctgcagaatatagggtagtttcagaaatcacccccagcttttctctagacttggtgacagctgatgctttcttctgactcttcctttcctggagtgccgaccttcctgcaccttcctcgctgttgtcacatggaaaggctacaggatgttagacaatgtcaacagtttactatgtctctggcatgaaataaatggaccaaccagtatctgagaatattttttacttggctaattgattgtatgttttcccctactatatggaagttggacttagcctttctcatgttaaagattattcagaggcaggacggccatggctgttgtctttgggatgtccactgtaaaatggggagtcatacctacctcacagggtagttgtgaaatttaaagaagctaactttggtaaagtctctagcacagtgcttggcacatagtagggtgtgttaatttctttcccttccctgcaggccattcacctgctcttccctctacgattttccataacacagtttttacttggaaccagccttctgccaagagtctcagtttggttgtgtactcctacaactactatttttggcttgacttccctctccagctcccagtggtccagccacacagtcgcctattgtaccagctggttgatgtaagtctatccatcctcagaggaacctggttacttaccagtaagctgtgtttctctgaaaatgtagcatccttggtaatgattctagtcccccccatcccctttttttttaaaccattgatgatgaatcatctctaacttccttgtgtgatcctcaagttggaactggaattagagaatctaatttgttgataaaggtctcctagtaagttcctgagtccaggacacatggctctgaccaggtgaaatgtggagacccatgaggtaaaaattcttttcactactctgagtgacatgagacttaattatgtaaactgtcaaggatgatacattttcaaagaaaaataagttgctggtaagtaactgggatttttgagtaatttttctgtaagtcaataaataagaatcccacgtttgttttcaccaacaaatgcttttgcatttgaatatttcataaatgaaaggaatatggttgttagttacggttaatagtaaagaatgggataggtttctagatactagaaatacagtgaggtgggggtagaccatcagttttctaaataggtgctctcttagcttgaaacagaaattgaagtctccattagagaaaatcatcaagtgtagtctgttcattccagaaacattttttgagtgcctactgtttttttgagacactgtgctgagtatcgtttcaagacagaggtgaagccacacttggctcagctgcttttgccctcagagagcttatttcttgtctcttagtgactctcgcatgactcagaactgaggcgatgggatactggggtatgacagaggtatgggactggaagtagtacgtagtgctgcagcctgtgccttaatattttctgtgcttccataagttctttctgttaatgggaaaaatcagctgacttcattcagaatttgcagagtatttgctgtatcactagagcaaccatagatgaagcaaagccaatgctccttctttgtcatagttgaaaactcattctctaacccttatgtgcgactttggagttaggtgttaaagcaggtgaataagtcagtcggagaggcagcccactccccattaagctttcaaagcacttcttccttcagaagccacagaattaatttattcctgccaaatgttaaaaaaatcggaattttttttttttaagaaatgaaaattgttttgtactttaagtcactggatagtatttgatactatcagtatcagtaattatattcagtatattcggtaattataagcacctccacccccaaatttacccagttaagtagaaagaaggaaagaagacttggtagtattaataagttgttgttggaggagatgctcctattacattatgtctctattttctttttttttttttttttgaggcggggtctcccgtcgtcacccaggctggagtgcagtggtgctgatcacaacttactgcagcctttgcctcccaggctcaagtgatcctcccacctcagcccctcaagtagctgggactacaggcacgtgctaccttgctgggctaattttttgtgtgtttttagtagagacagggtttccccatgttgcccaggctggtcttgaacttctgggctcaagcgatctgcccgtcttggcctgccaaagtgctgagattatacgcttgagccactgtgcccagccatgtctctgttttatatagtaaatactgcatggctgtaattgttattttaccccatgagcattgcaagggcaagaactacagtgcagtaattaattaagctcacgggtgttgcagtcagactttaaatccaggttttaatcctggttttgtaatttgctacctttctgactagacaaattgtataacttctctgaacctcagtgtcctcatttgtgaagttttgatgctaaaactacaccataaggttgtaatgaggaataattaagataatatatacaagcacttagcacagtgcctgcaacatagtacgtgctcaataaatgttaactgctgtttttattggattattggatggatggatgaaggattgtatcagaggccaaatagcaggaaggggaaagattgtgggctctggagtgttagtaaactgttcacaccttcttgtttactcgagttaagagccttattagtattattacctttatctttgcctttttggaaagcaaattaatagatgaatcactgtcacagttcttcctaactactccatccttaaacctttacttccactaaggaaggaattatttttgtgctgcagtgactgtgatgattgagagattatcccactggcctgatgcaacccaatgctgagggtcaggtgtggcccatatgccagatttctactctgaatttcaccacaactagttctgctatttttaagacattttaaataaaaaatttaaacatttaatatgtgtgtgtgtgtgtgtgtgtgtgtgtgtgtatatatatatatataatgatttttcagtttcattttactacacttcttttttgctctattttcattgattgtggattatgaatgtatgtctaggatatttttgagaaggctctggaggacttacacctgggcagtagtactctttaaggaatctcggattcgtagacagggtggaaagtgctgtggttatttaggcacagagttataccatatttcagagaatcaaagatgcaaaatttttcacattttaacattggaaacaacatgcatcttagaatcactgtgataagaatggattatacctttgtttagttggcagcattttttcttagtgatacagaagataatgatgtaccttgaaatcgctgtcatcatagattgtatgaaatatggtgtttcaagggactatgtgcactgacacattttagcaatatggtatcatggcaatataaggagcgggaagagcaggggctccagagtcaggcttttggagtttacattctagcccgagaaccatgcgcaactttgggcaagctacttatcctttcttttctatagttttctcacctgtaaaatgggggctaagagtagtacccatcttacagtgttgtaatgaggattaaatagaagacatgtaatgtgtttaaaatagcctagcacatagaaagtacttagtaatcagctaatattattaattcagagattacagttttgcagaatagacttaaatttactttgttgtgattgaaacacaaacccattacccttaatatttatgtatcatctgataatatcagcaattgacacttagattttaaaaaaggaagataaggaagattaactgaacaagttttcttattctttttattttattttattttattttatttttttttgagacggagtttcgctctgtcgcccaggctggagtgcagtggcgcgatctcgactcactgcaagctccgcctcccgggttcacgccattctcctgcctcagcctcccgtgtagctgggactacaggcgcgcgccaccatgcctggctaatttttgtatttttagtagagacggggtttcaccgtgttagccaggatggtctcgatctcctgacctcgtgatccgcccgtctcggcctcccaaagtgctgggattacaggcgtgagccaccgcgcccggcccaagttttcttattcttattaagtaagatttaagtggcttatttggtatacatattactattctgtgtggttggaatgtaagagaaatgaaggatgagctaggaaataaatgtataggaaaagcatggaggagttaatcagatcgctgaatctcatataagtacagtttttgattcttactgatattggtaagtatgggaagggttggaggttttgaaaatttggaaaatttgaagcatttttgcaccctaagtaaaagaaaagagagaatcctatttggtttatatgaccactttactgtcctaggtaaggacttttttttcaaaattaaatatttgggtaggcagcatctctttacaatatgatggctacagcttaaggttgtccttttattcttaagagtcaattttattttttctgttttagtgttcggtgaccagtgacttggattttccaacacaagtcatcccattaaagactctgaatgcagttgcttcagtacccataatgtattcttggtctcccctacagcagaattttatggtatgtaaataagatatcttctgcttatttgataataacttatttgaagtaaaaaagcatagataattttattgtcaaggtgaatcaagacagtataaattctaaaaagctatcaaaataacacttgaagtaatttttaaaaattgattaaggtagtttttttaaggcctgtgcagtattactcttttcctatactctccaaaggaaaagtgttaagcagtaaccatacctaggattaaaaatatttaaaagatgggctgggcattgtggctcatgcctgtaatcccagcactttgggaggctgaggcagatggattacttgagtctgggaattggagatccacctgggcaatatggcaaaacgctgtgtcctcaaaacaaaagttagctgggtgtggcgcatgtctgtagtcccaagctactcaggaggctgaggtggaaggatggcttgagggaggcggaggttgtagtgagctgagattgtgccattgtactctagcttgggtgacagccagaccctgtctgaaaaaaacaaaaggaatgaagcttatagttcttatccttaaaaaagtcagtgtcctgtaatctaagtctgactttgagtttttgttaattgagaacttattatttaaaacaactttaggtagcattttattgtgataatgtacattatggtgtaaaattcatagggcttgtgaaacatgagctgaaacatagttccattgctaactctgacgcagcagcttttgtaataaaatgctaaagagaaactgaagcatgtgttcttgacttgtattgctaaataaaaaatatatagttgtattacttaattgatatataaagtgggaaggctaataaaatgttgatagaagtccttggtgaacctgctttggggaagtttttagtaacttttatactttgtacagtagagtctcaacttcagttactcttctgtagttgtataaatttgagtgtagtttgagaattacattgcatgaccttgagggaagtttcatgttttcacagactgaaattctggaagaatatattatttaaatttgtcattaatttatgaatcacccatggattttgctggtctagcctgggtgacagagccagaccctgtctgattttgctgaccaaagcagattaggcacacaatctttattgtaactaagatacgttttggagagcaggatgatgtggatatgattgaaaagacaaaatatctaagtgtcacttgattttggcttagatagaaagaaatcaacccctagtttaaatgagattagtgcaagtgaaatgagatgaaaacaggcctctagtgtgtacataagcttgaatttgaatatatgaatacattaaaacctgcattttaattatatgctccttttatcccactttttcttttccttgtctgtctacggtacttgttatctttatgaactctccagttgtcctaaatttgttacctagggattatttgagattttttccacattccctggctgctactagtcctgtgtgtggcctttcccagcgtgttcttacctgtgggccatgatcatagtgctgggccgagcctacgccatctcaccctttaaagagcttctgtgtcctcccagtcaggcatagccagtccattctgcttattataattatctttctaaaattctgttttcaaccaatactcttttcaaaggcctcttaagacttagacttgtcttcacttggaattagtctctttaaacatgtacttcagaactgtccagtctttatctataactgctgtcatttttattcttcattgaaaatgctcttcctcctatgaatgtcaactttctttaccttgtatgcttaactattctattctgtcttaacttttgtttgattggctattcttctttcttctctgttgtcttccaaagcttgataaaaactcatctatcatctttactagcactgtcaactttgatattcatctcattcatctaaatattgaaatattttatgtttttgtagaatgttcttattttacatataagtacatatccttttggcataaactttaatggactatcaatactctgtatccctagaattttattatattggatgtcttacatttttttttcattttaccttaaactattacaagttaatggctgttttaataactggatcatcaagtggagtttgggagtcttagggtagttttttggtatgggtctttatagttgatcttgatacaccatatttcatcatcctctattaacatctaaagtgcgcgttaaaactgatgatggctgggcatggtggctcatggtgccagacacctgttatcccagcactttgggagccaaggcgggtggatcacttgagctcaggagttcgagaccagcctgggcagcatagtgagacactcgtctcaattaaaaaacaaaaacagaaaaacagacgatgtcttctcataatctagagcatctttttctttcttagaggtatgtacaataatggtggtaagttttaatgattgatgacagactaagtgcattattttttctagaattaaattttttttctgtcaacagttttctcagatatgcttattggtgagaggggtcttttattttttgataaattttagagtttgctctctgactagtattttaaatctggagaactgggtaaagacatgtacacatgaaaaaaaggttgtattttagttatttcacatttgatacttagagtaaacctgctttttaaatatatttttttcttttaggtggaagatgaaactgttttacataacattccttatatgggagatgaagttttagatcaggatggtactttcattgaagaactaataaaaaattatgatgggaaagtacacggggatagaggtgagccatatgcttcttctcttggaaaaaggggctaaagaattgagagacacttttgtttatgaagcatatagggcatgagaaaaattttaatattaaaattaagttttgcactgaactataagggactgaacctgggcccaggcctaagattttcagtattttccctaactatttttgggaaaattagttaggaggatcatgtttgccacctgataattatgcatagttggttataacataatggcaataacatgtaaattaaatagaacttgagtgtatgtagattttaagattctttagtagtaaaattaattaggattggggctctgcttctgtggcatataggggctccaacagcccctactactaaaaaataccgctcctctagggactacagtacctgaaattcaatccttagagtcatggtttcagttttttgtgtccaggaggccctgttaaagtgtctgaaaggataatatttgtaatatcatcatccattgtcattatcagcagaaagcagggtctgaaatgatcagttaaaggacagatccattgtttgggaggaggccaaagccacaatcagttcctagaagattcagttttctactagaaagttttggggaggggccattctacagaaagtagcaggtattaaagataagtttgcttcctttgcctaacaccagtcctgaaaatgatcttatcttttcctcccctcatttcaataataatgttttagtgccagttactaggctatgcctgttttgtccaagtagaaaaaggatacataaacttttgacattattgcttctcctgtgtgtttctgacatttgatgcgtttcagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatggtatgcctgattattaggattctgtgtagccattacagctttctttcaaaaacagtagaaagagtagaaatagggcttagtttcttctttctctttttctttctttgttaaatatcacattgatagcagtataatcaaaattaacatattatggccaggtagggtggctaactctgtaatcctagcacttgggaggctaaggtgggcagattgcttgagcccaggagttcaagaccaacctgggcaacatagtgagaccccatctctactaaaaagtaaaaaaatgagacagggtgattgcttgagcccaggaggtgtaggctgcattgagctgtgattgtgccactgcacagtctggctgggtgacagagtgagaccttgccttaaaaaagaaaaaaaaaagcataatacttgcaacattatcttcacttgagtatctttgtttcctttggctagcaaagaacgtaattttttgtgtgcttcactgggcatcattataatatataacattgatattcaccgtgtacaagttgtgtactaatcacttcttatgcaccagctcacttaatcctcataatggcttcatgagatggatgttattatcagatccattttctttttcgagacggagtctcgctctgtcgcccaggctagagtacagtggccccatctcagcttactgcaagcttcgcctcctgggttcacgccattctcctgcctcagcctcccgagtagctgggactataggcacctgccaccacacccagctaattttttgtattttttagtagagacggggtttcaccgtgttagccaggatggtctctatctcctgacctcgtgatccacctgcctcggcctcccaaagtgctaggattacaggcgtgagccactgcgccccacctgtcagatccattttctatttgaggggatagatttagagaggttaagtaacttataagattacactattgggaagaaacagagtcaatagttggtgttctctacttggcaaagctcatgctaaggcagcatagaatagaataatttggcgggtgaaatagcaatttctaagaagcgtatttcaaaaaagagaaaataacaaaaaggtaccttattgaccttattgatcatctagttcagctccttcattttacagatgagtaaacagacccaagaggtaaagtgatcactctaagtcacaaaatcagttagtagcagagctgggagtagaacctaggttttctgcttcccagtgctcttaaagcttttcatatttgaatttgaattttgtttttgactgactggcattccacagacatgtaaagtatgtgagaagtaagtaaaacacaaaatagagatgatttcttagaaaatcagctttgttatagagacataattgggtagagaaaatgaaagatcaaaaacttgtttacttccattctttttttcctttcatattctcctgtttagataaagaaagccgcccacctcggaaatttccttctgataaaatttttgaagccatttcctcaatgtttccagataagggcacagcagaagaactaaaggaaaagtaagaatttgttcctttgaggcaatcttgccctgtatggtatgtaactcaccattttggtttattacaaatggaatcaatgtagcggatgagccactacactttgtttacagtgtttgcaagatacttgaagcaacatgtgtacttgtggtactctgcattacaaaatatttcctgtagatagaataaatacatatgtataatttatatatacacataaacacacacatatacataaaatttacagtagtacatttacaggaaaattatctggatactggtagaaatttgatgtttagttaaaactaatataatatccttttccatgatatatttgaatcatatcgatgaatttgattcttgataacaccatgcacaatatttagttggctctttatctaaatagagccattcctttatgttttaggcaagataaacatcaaaagtaacacatggaaaccttttagaaactgttttcaaaaaagtgatttttgtttcatgtttacttaaatttttcttggttaatgtcagatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattgtacgttttcattttctatctttgttgtggaattatgttttaaagcagcttggagagtgctatcatttattacaactagaatttatcatttaatcttgtagttactttgacaaatatctttagatctcagtattattatttttactttgttgcttgaggattgctctgaaatcttttgcatgtatttcatttggactactattttccactaacatggaattcaggtcagtgatttttttttttttcccccgtaagtgctaactaagcagttgttttacaataaaactattattatgaactggtagcccagagcaacattgctgatattgatgaataaattgtgttaaataagaaattggccaaaaaaatgttcaggtaagtgcttgtcgatttaatgtaattttattttattttattttatttttttgccctctgggtaagttgttttttaaaaatacctgcatttgaatacaagtaaaacgtaaaatgttcttagtgaaaatgtaagtatggtatatacaaaaatacttttttcctctggcatttctagggaatgtggtttcttagtaaattttcctggtaattgaaacttctaagcacagtttaaaagcgtcaactacttttgaggcacatattttgaaaatgtatttaatacttcctaattttcctagagtagtttgcttattatagtttggtttttcctatattgtattttagtacgtctgtctatggcatggtaatttctttaggggctttgacccattgtaaaatcgttttcccctgtactcaacggccccacattgctttgttgttttgagttcctgtgcctaccattgacagatttattcctcctcgttcaccaatgataacgtctttttaagcttgcccaagcagctgtacctcacctcctcctaagtgtttgctctacttgtttttgctgcagctaataatgtgttgagtgatgtaatacattggtctggaaatgttaagttttgtttttgtgtgtgacatattagtggtttagttatcttaaaatagaaaaacttgaagtagatttttagaattatcattatattgttacctccttgctcttgttctaactcctcatctagtttaaattgatttggtagttttaaaaattcgtagtaagtcatccatttgttgcttcagccattggacagttaagcccttgaaatagttgtcttgaatttcattttaatttccctttacacaccatgaatttatgaattacatcgagtttttattaagactattggacattgtatgtagatatcactatagcaaaagaattgagtggaagcactattttggagtcgctgaaaggtccttccaatttgttgtttatagatgtagcatttaataaagtttttttgctgtaaaaatgaaccatagaagtaggacactacttttaaacattacaagttttttattgtgccattcagagatgagtaatttttttttttgttaacttgaatcttgaacctgttacttacctcattctagctggggagaacagtggaatattttactttaataaagttggctttcatggggctaattggctgcctccttgtagcatttggtggccatgggtccaactgtgctaggcccacttttacttctttttacagtagagccagtgtcttcatcattcagtcctattgagttctgtaggaacttcattgttttacgttaacttcgacaaaataccacacatggtatatttttcttaggtaaaataagttactaaataaatcattaaatatatataatcatgcattacttatggacggagatgcattctgagaaatgtgttgttaggtgattttggcattatgtaaacacagagtgtacttacacaaacctagatggtacagcgcatgacacacctaggccacatatccctgcagcatgccactgtactggatactggaggcagttgtgacacattggcaagtatttgtatgtttaaatcataataaacatagaaaatatacagtaaaaatacaatattatcttatggtaccactgttgtatatgcggtccatcattgactcagatggtatgcagcacatgactgtttcttttttgttgttgttgttgtttttttttttttttgagacgaagctcgttctgttgctcagtctgaagtgcagtggtgtgatttcagctcgctgcaacctccgcctgctggattcaaaggattctcctgtctcagcctcctgagtagctgggattacaggcgcccgccaccacgtccagctaatttttgtatttttagtagagatggggtttcaccatgttggccaggctggtcttgaactcctgacctcaagtgatccacctgccctggcctcccaaagtgttgggactacaggtgtgagctaccacgcctggtcgattttatgttgaatatagcattttgtgctttagttttctggtctcttatcttggggatacggtagtacttcctttacagagctgttgtgaggattagatgaattaatgtggggaagatacttaaaatggtacctggctcttgttagctattgttgtttttaaaataggtcatcattcacatggtttacgtttccaaaaagtattaatatgaagatgtgctgggcgcggtggctcatccttttaatgccagtgctttgggaggcagaggtgggaggactgcttgagcccaaaagtttgaggctgcagtgagctatgatcatgccactgtactccagctgggtgacagagtgagaccccatctcttaaaaaaaaaaaaaaagtatgaaaatgtgtacaataaagtctcccaatgactttctgttccccatccttcgaaattttacctcactcttccacttttattagtagttaaccacttttattggttctcgtattttttttacagtttctttgacaagtataaagatacccttttttctgaccgtcttatttacatggtggcatagcattcaggcttttccctttttttggaataaggataggatttaatattgtagctattagctttatctgtatagctgcatagcctgtgctgtaggaggttccagaacttactggactggtctctaatggtgaccatttggattattttgagccttatgctgtaacgatgccacagcaaatatctgtacccatttcatgcaagtttaagtttacctgataaactctatttaggtattgccaaatgacctgccatatggggatgcagcactttaaagccctaccagcaaagtcagagagccctgcccatagaaggtgttaccaaactaatttttaaaattgattttataagtgaaaaatggcttttagcattatttttaaattcttgtttttctttgttttttaatctctgcttcccacaggtgataagcattgttttaatatctggatttaattcctttgaatgagggtaggatcttcctatatgttgaagagtgaactatttttattctttgtttttttgattttgtgagacgggtctcgctctgtcgcccaggctggagtgcagtggtgcagtcttggctcacggcgacctctgcctcctggattcaagtgattctcctgcctcagccttctgagtagctgggattacaggcgcgcaccacctcacctggctcatatttttatttttagtagaaacagggtttcaccatgttggccaggctggtctcaaactcctgaccttaggtaatccacccacctcagcctctcacagtgctaggattactggtgtgagccactgcgcctggcctatttttattcttggtctcatagatctttttgatgtcttaggagctttttatataatcaggaaattaggttttttgaaatatgagttgcaatttttttcaagtttatttggtaaaagtcccataagcaaagtttaaacttttttttttttttgagacagtcttgctctgtcgctcaggctggagtgcaatggtgtgatcttggctcattgcagcctttgcctcctgggttcaagggattcttgtgcctcagcctctggagtagctgggactacaggcatgtgctaccacatccagctaatttttctatttttagtagagatgtggttttgccatgttggccaggccggtctcaaactcctgacctcaagtgatctgcccgcctcagcctctgaaagtgctgagattacaggtgtgagccactgcacctggtcagtttaaacattttttaaaaagtagttgaatttatcagttagctttcccattccaagattataaagcgtttctcactttttctagtatgttttaggcttcatttttaaaaacataaatctttgatccatttggaatttaatatgatgtggattataaatctgatcctcctcctcacttccaaagttgcctagttgtttcatttactgaaaagttcatctttcactaccttgagatatttccttcattgcctgttgccatatgtatttgggtttctcaacattgtgttcctttagtctattcatgaggcggtattcagagatctcctggcaattcttgtttttctatataaatttatttatttagagagagagatatatatacatacacacatacatacatacacagggtcttgctctgtcacccaggccagagtggtgcagtggcacaatcatggctcactgcaaccttgacttcctgagctcaagctcttctcctatctcagcttcctgcgtagctgggactacaagcatgcaccaccattgctggttagttttctttttctttttttgagatagggtctcactatgttgcccaggctggtcttaaactcctggcctcaagtgatgctcctactttgggctcccaaagtgctggaattacaggcgtgagccactgcacccagcctatatacatttcaaaatctgtttgtctagttctttttaaaaaaaattagtggtatgtttattggaatcccattaaatttataaattacctaatttctttttaaaatataagggttaagtctaactaaccataccatcacctctctttttgtgtcctttaattatctcccaagttttctttatgtgactcttgtacattttttgttgagtatttctaggtattaaacttgctgctgctgttgggctttttccacattttgtcttctgttttgtaccctgctaccttactgcattgtcttcatgttttatcagtttctcagtacattcttaggagttttccagctgtgtaattatactgtctgtagttattaatggttttaccttctatccagtctttataactgaagcttctttctcttttctacctactttatttatttattttttggttgggcgggggaggggagtggggaggctttctgataaaaaccagaaagcctgctagacaaattctaaaagagctgtaactgtcttttctacctactttagcttgtagtttcagtacagtttaaaaattagtagtgctggtagatagagataaatgttctgtctcttctggatgagttttagaaaattattctttacatccaggttttgagatttatttgtatgaagtttatgtgttctctaatgttcgttttatttttgttaatattcttgttctctatatatttcattgtcacttttttgttaagaagtggtttttgtattttaaattatgtatttttttcttcagagaaccagctacaatgttagtttaactcataaacagaaaaacagttgtattagggttccttaacaaacgtaatccagcaccagattagtattggtgttaggctgtctggagcctaattgctcacacgcttccaacagaatctgagccttgaatttttcttctttaaaaacacaactttgtccttttttttttttttccttccttttctcagtctttaaatatggctttttacaaatatttaaaagatgatttgttttgtggaaatgtcacttgtttttctccaaattaattttcatcttgtataattctgaatagtccatttccagcatttcccttttgtatatgaggaagtaagtttttgcaagaaattctgtgaagttagcactgctgtcaatgttcacactcttttcatgtagcagtgtacccgcatttgaatctttctgttacattattttggaattttaggtttcaaacagtgtatctttctaaaatccgtttttttgacattgtaattttctgtggcaagacactataagctctcaccaggttcttggattgggtatgattcacaggaattgaatgttagtaatctggggctgtcctctttatcattaaaaatgagtttttacaactggtgacaagctgattgctgacctttctttagaaatcaagaaattgtatgtcagaattaaaaataggaaatctgatttaattgtagagggtctatcaactgcttagacagagatagtaagattcaatttaagtaaatgtttataaccatagttatgcgcatcagttttacttgcaaggagaaaaatgagaaatcacgactcattccagaatttactgtttttccttcctgcttaatggttggaggaggaggaatggagaatacgttgtgatcattcagtaagagcctgaaggaaagttgtatgaagtaacgtaaaccacatattagcaagattataatccattaattgacttttccagtggaactggaagagtgaaaaagtaagtattttatagttatattagattctttgtttcatttattttgcagcttttcatgcaacacccaacacttataagcggaagaacacagaaacagctctagacaacaaaccttgtggaccacagtgttaccagcatttggtaagacttagtgcctaattattgcagagggtacttgagaggactttgcactttggtggaggtgatcaagtcagcgttaatgttgtctgcacccatgctgttatgggccatatgatagacagagctttctcacccatgctgtttcttccatagctcatgtactgcacattttagtccttcattaatgtggtattattctttttaaaatctgtgttatctcttcagcaacatttcaaggtctttaggatcaaggacttttatgccttccacaaagtctatgagagttgcaaggcacagatagtccgcttagaaagcaggcatgcagtaatcatacgcagtagatcccaagcccctctcatagccatatccctctacttgagttttagatgcactgtcatttcagctatgtgataggttccacagattttttttttttaaatgatggagaacaggtaaggggttaggaaaatatgaggtagaaaagttgtttttacgggtactctcttatttttaccatttcccctctttgaaccaagccactcctacctaggaactaaatgggtatatattgcctgttggattttggacagaagattcattgaatggcacctgcagaaggtatcctatttttaagcaatttggtttttgtagaatgaagtataaaactgttggctgctgtcaaacttctcccttctatttccactaaaaaaatggaattcttgagcacttgttttataatattctttacaaggcagaattttgtttggctcatctaaaatgtttctgtggtctctataaaccatgctcttaccattgttttatactttgagactggattattgtacttcataatgcaagatgtaaccctcacagtcttattcctgaagttcctttcatgtcttcctgtggctgtgtatccttcacaggaaaaaaaaaaatgttggatgttttaatagtaagacaggaagtgacaaagtcaagcatgtttgtgttttgtgttcttgtatagttgaaatattaacacgaagaggtatgcattatcttaaactctgcataggtagtctgactccgtgcacattaggtatatgattcttttgtaaagattcacttttaaatagactgtatgtccatagagataattacctttattacatgaagtatatttcatactttatcttcttatattttaacttaattataactagctaatatattttacaaattcacaatatagaactgtcttgcctgatttttgtaccattctaggaatatatagaacccatatttctctttctgaaattttcagactatcaaaagcatatcaaaacaagtatattagacatgcaaatgattatttgtgataaatggataatgtgatacattttttgactaacctggcttatatagtatttttttttctcttccatcaaaatgagttttagaactttgccctgatgttgacatttttcatttcgtaggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctggtaagacacgtctaataactgggttttactgttctgtgaaagttcgtgttgtgaggattaaggatgtataatgcattgaataattttcttagttggttagtttcagtataaagaccagagttatctcaagaatgtgtagctgtgttgtttctgtttcctgcctgcacccgtaccctagcttgtggtgtttgccctgcttggttgtttgaaagctgatgatgcactttattctcattctttgtgccattttcttttttcgatctttctcaggtagggaataatgaaatttggatcatttggtttaatattcttgacgttctgtgagtagttttgttggaaaatgtgaactacgatgggttagtgttttgccgattggatttgagttgtcctcatcttttcgcttttttcttaagcacaccctgaattgtacttcttaatattatttccaatcatttcttgaccagtgcttacatttggttttgtagaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtaagatatccgataaaaatgttgttaaaagagcttcaaacatagagataaaactgtactcgttgtccaaacaaagaaaattcttggttattactaagctcctattcatctcctaaaaataatttttttttcttcccaagagggaattgaatgaaaattattctccaggtatgttacagaatttccccaatgtagacttttaaggttagatagcagtagcttttaaaagctagtaagaattgaaatttagcatcacagttcttgaccagaatataattacagctacccctaatgtgagggtagggacagcgatgtgtgttatatccccagcatctagcagtgtcacagaagagacaaagacatttttctggtatgcatgagtataagaatggtttgcctaaataagactgtcctcatggctctgtgactgtgcctcttgtcaggtgtatgagtttagagtcaaagaatctagcatcatagctccagctcccgctgaggatgtggatactcctccaaggaaaaagaagaggaaacaccggtgagtctgtcatgaagctccttagagaagtcaacatagatagacactgcaggcaaggcccttcctaagggctgttgttggtttctacttagtctatttttaaaaaaccttaaactgccatcaaaaacagaactatggaatcttatttccactgggaattgctagtatttttctataagaatacagcagccttcagtatatttggtatgttaagagtcataaaaattacggttaccagactgcatttgggttaagaaaaacatatttagtagcttttgcagtaaaggttacgtaaacaattactttaacatcatgaaatgttctgtgtttctgatgtgagctctgggccagtttttagaagtatcttttattccaggagcttgaatttctaagttgtgtggcattgcattccgatactcctaatgggtaccctactttaagcttgtgtgaatgttattttcaccttagcgaatgttttcaggctgatttgatttgtaaatgtagaaccagaaaaattgatgagtggactatctgctctaatctccccatacctctacctcacatttttaaaaatgagaaacctaattgggtaggtaagggtgcagttagattatgcttcttaaattgtgtacaggctcatgttacattttcttactttaacaaaaataagtgtagggccaacccaatttagaatttttttaaagtcaagtttttatttttattttttaggctttaggagatgcttctgaaattttggttgaattatttctgaccacgtagttcacttgcttggtttatgatgcctgaaataagaggaagtgatcagtgtcatccacatcagaataaatcaacaggcagacacacctgctgctttgtgcagcattgggagctcaggagttctgaccaggagtcatggatgaacacaggaaatgacgtcttgccacacctggggcagcctgagccatcaagctgtttgagttgcttcaaactgatggcaaatataaactgggcagggcatgactcttggctttaacgcattcctgtattcatggcacagatagatccagttaagcagctctctgttggatttgtagcttcccgcagaaatttggtttaattttctttgtgtttttgcaggttgtgggctgcacactgcagaaagatacagctgaaaaagggttagcatctttccattcctctcattattagcttaacaatatctgctttgttttcatttgttttagaatagtaactggattagagcttgggttattctaatataattgccttctgtatgttaaaccaatttgaatggaggacgtctactgatgaatccccagtcaattctcttctgtaaagtggaggctgactggaaggtgtgagtccgagtcagttgaaggagaagtgctttgggatgggactgagatgtagccgtgctttagatggaactcataagcacttgggggtgggagagtattttttgccatcgttgtggaaaccggaatatgtgtaatgttatgcattcagtaaaggcaggccagctacactccacaggtagtagggaagaatgtactcgtgttaattgtgtatgatcgtttccatctccctggattcattggcctgcatgatgttatctttactcagacggctcctctaaccatgtttacaactatcaaccctgtgatcatccacggcagccttgtgacagttcgtgcccttgtgtgatagcacaaaatttttgtgaaaagttttgtcaatgtagttcagagtgtaagtatttgttgctttgatgcaattgcatgagaactaaataggtctttggttagcaatttaaaaaatcaaaaataagaacatgggagcactcccttgtttacttgacgaaactcatcaatttgacaagtatttattgagccccatacctggccctgtgccagtggccagtcagtcgcctgggtgttgtgtgtctgtgtgtgtgaggcaggcaaagttgtattaaaggaacctgaattttatttttttcttctttgatcatttaaattaagttgtattaaagttggcacagattgtaagaggaatctagaaggagctacccaaataatttttgggcaccagaaatcgtcagagttaggagggttttagtcttcagattaaaggaatgtagcataaacatagtagatttttataattgtttaatgagactgcacatgaagcttgggtattaagggagtgtttagaactaggggctattggaatggtcaaggaccatggatattgttccatccagaccctttattcacaccgactttaaatatatacactatttaacaaatgaaaaatggaaaaacaactcagtggtggtctttttcagtgcaaacagctataacttcgctgttaatacttccatttccttgtctatatagtagggtaataaggtggttgtgagggttgagagtcagtgagatgcccagtacagcccttgccacgtatctttctgcaagtttttgcatacggtgatgagtgaagaacctccaaacctctctctgaaggtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaaggtgagcaacaagtcacttctggaagattgtttgtagttagctatttagtgatgcaaatgtcaggattgccttagatgatttagctggggcaaaaagtaaaagtgatccttgaaaggtgcaactgatgagaaagtgttcataattttctctgggtaatgcacccttgggcagtctcttcagttttatatagagcaccatactctgatagtaggagaaaccagcaaattctgagaagtagccattcatggtatcagatttaggaataagaggtattttttctacctgtttaatagattatttgaatttgtcttttagttaatagtttatctagtatccttaatgcaaataaatggtgatttctatcaaaatatagactcattctcatcaaagagtatttatagattccttgctatgtagagtaaaagatgaaatttcccatggcaggtttttatagacttacttcctgtttgtttaataaccttgaaaaaaacccagctaggaactatatatttttagtctggggaattgtatgtaggtgagttaactctatcagccaggaagtaatgattgagcaaaagacacttcagcagcatgtatttgggaaatttcgtatcttttttttttttttagggcagatgataagttctttcttcagtcagttctttttataataatagctgacatttattaagcatgtaaaaataggcactgttgtgagtgctttagtggatttgttcattgattctcctaaccctgtgagtgaggagtaagtgttaacccccattttacgggtaaggaaacccaaagtcacaagacttgtaaaggagcaggagatcaaatcctccaggctcccattaacctgctcttatgctctgtgtgtgtgtgtgtgtgtgtgtgtgtgtgtgtatgtggggtgtagagtagacatatctttgttgttggaccatttatttataattgagaacacacttcttaccagcaagtccccttctaccagaaattctgctccaggcaaaacattgaagttctcttggttttgtttgtgtctttgaaccaaagtctgtgtttatggcagagagttgtgcagggtggtgggctttttgtttttagctttgacttttttcttttctgttagacatttagatgattttactctaggcagttatatgtccatttaggtcattggatcacagtgactctggttccgtttccgatggcatacctgctcagacctaaaaatctcaatttcccccccaaacaacttaccattgtactccttcctcactttgttcagaaagtgggcaattctattcatactgacatgtcctctttgtaagacattttttaactcttaaaaatgttaaactatgtagataccttttaccagttgggtttcatttgcatagcagtatttagctaatttagtttctgatcagaaggctatttgggaagttcacatgacaggtgagtggtaagatggtccagaataggaatagataggaagcctttgtaaaggtcacaagaaaattttggatgaaagcataatactgagccgtgatgaaaaattggcatatgttggggaacacatccctaaatacataatttatagcaatgatcatcttggcatttctgctgaaaatagactactggactgacaagatcatgataacacctcccggtagtttcttgtgttatcctgaggaggtggggtggaagaacaaatccaaagccaagctgctatgggcttcagagggaaccaacgccagtgaagaatcagggggtgtcctggggctgaaaatggaataactattaatagaatcacttggggaaagtagagggaaaggtatggagaggaagagtgatttggtggtgtccttatattaaattcaggttgaaagtgaattactcgaggaaatcaagggctgaagatacagggaagccaaaagtgcagcaggcctagagcaccttgctgaacgatggtcattgcagaggaccaacaccaccaaaaggttttctgtaagacagagattcttatctgctgtataaggaaaacataatgttcatagccattctcagcagctttcacgttgactgaagctgtgtgcccaattactgccttagaacaaacaggtctgaggatttacagtgatagcttttgttttcattctgtagtctactttgtccccagtccattttcaccctccttttttgatgatgtgattgtgttttattctctagcatctattgctggcaccatctgacgtggcaggctgggggatttttatcaaagatcctgtgcagaaaaatgaattcatctcagaatactgtggagaggtaaggcactgataacctgtattcaggtggcattgtatatactaactttactttattttagattgattttattaggtaagtctgtgggtttgattggaaatgaattgccataaactgccttttcagcctggacttctgcatgtttgtggatttgcatgcttagtaactggattgtgctgggcgcggtggccgactcctgcaatcccagcactttgggaggccgaggcaggtggattgcttgagctcaggagttggagaccagcatgggcaacatggcaagaccccattgctacaaaaaatgcaaaaattagccgggcgtggtggtgcatacttgtagtcccagctacttgggagctgaggcaggaagatcgcttgagcctggaagagcctgggaggttgaggctgcagtgagccatgtttgtgccactgcactccggcctgggcgacaaagtgagaccctgtctcagaggggaaaaaaagcaaaacaaaacaggattgcttggttctagccacatttagtttttctcgtctagggacaacagggagaacaagagctttaccatagagtggcctgcttaatccacaacctagttgataaggacctagaaacaacaacgtcattagttattcaccttttaaattgtcgacagtgttaccgaaaaatcataggcctagcttcagtttggaaccagctcacccaaattttttttttttttttttttttttttgagacagagtttcgctcttgttgcccaggccggagtgcaatggcgtgattttggctcaccgcaacctcagcctcccgggttcaagcgattctcctgcctcagtgtcccgagtagctgggattacaggcatgcgccaccgcgcccaggtgatctgcccgcctcggcctcccaaagtgctgggattacaggcgtgagccactgcagccggcttttttaaaaaactcagcaaaaagactttggttcctcactttctccaaggataattacttcaggatgtggaaatatttctgagttggggtccttagtatacagttattctttattactgcgcagaccgaatgggaaagagaacttggctgtagtgaccctttttgttgcgttttctccagaaggtccagtattcactctgtgcgcttttgtgtgttctgtcaggcttgatcacctttatccaaaagaattttctcctgtgtctttctttttagattatttctcaagatgaagctgacagaagagggaaagtgtatgataaatacatgtgcagctttctgttcaacttgaacaatggtatgtttcagagctgggaggcagtgagttcctgagttgcatgtggcagccttctgccggccgagtgttcagaaacgagggtagaggtcagtgaggtgaccagaaggaggatctccttagaggcatggggggctgcttgcttgaaaggaactggagcaaggaattcctcagggagcagatagactagatgtgcaggaaaccgcctctctggagtcccggctgtctgtgggtggcactatcccagcatctggctgtgaaggggacgttgtgctcttgctgctcacctcacctcttgtgttcaggcagctcagcacacctcctccagtcagtgcctctgagcggggaaggaggggaggagcactgcatttgcaaccatcttccagactcccctggatattctcaaaggactgagaggaaaggcagctgctttcatagctgatgttctggggtagaatgtaaggctgtgtagggatgtttctggtgggattatgtgaaaatggaggtaggcttaacatgtaagatattcagagaaactcttagttggaacaagtagaataggtttttttctatgaatgctttatagacattataatgaaaaaaactaattttaaatgtgaaaatgcctattcgtgatgtttggaagaattttgtcagatttttttttttttttgctatgttttgctcagtaatgtatgtttatagttgataattaatagcttttgagtcagataaccatcttgaattataggtgattaattttaacctggaacaatagtgtgttcttccaaatgtcatttcttactgaatgaagctgcttgatttatttgcttttgcaggcaaaccctgaagaactgtaaccagttgcatttacaaaatcataataacttggtttacttataactgaaattattcactgggctgtgcttacttttttctttttagattttgtggtggatgcaacccgcaagggtaacaaaattcgttttgcaaatcattcggtaaatccaaactgctatgcaaaaggtaggtacctttgacgtgagaattggaactcccttttcagtcctgtgatgatggacttgaatacttctgggatagttgtttgttatgtgaacaagtcagttaaccccccgtggcccgtcttcatgctcactgacaccagtgtgtctctttgcagttatgatggttaacggtgatcacaggataggtatttttgccaagagagccatccagactggcgaagagctgttttttgattacaggttggtaaagtacatttctagcatgatctctaagggctttttctactggattgtgaactctggacaaaggagggtttttagttctttgcttcttttgacgggtcactttgccatgagcattagtggggaattaggttacactttcctgttatgtatttattatccatttatatattatacaaggcatgcttatttttaaaatagagtaaaatccatgcagaaagccccatttctcaccctgctgttgacagctgggtgagtcctagaccttctcatatcatgccgcatgctgcatgcttactcgtggagcgttttccagataagtgcgatcacactgtcctatagatttttctgcaacatggttttacttgacagtatactatgcatatctttatggattagatctacttaatttattgtttgctataatacttattacacatatcattaccattttaaaaggggctgttggcaaatcgctgtctgatcaccctgtggggatgcctgggacacgtgggccaccaatggtatctgtgtggttaggcattaggctgccagttgacactgtcaagctctgtgggatacatttaagtcagccattgaatacagatttaagtgcagtcagatgactgtctagttaatgaactcctgtagccccgtgtacatcccatccagaagctgtctttgagtactgtgttcttgcatattcaggctggcctttaatggctgttgcacagcagcgagctctcagtgaggcagacagggtctgagtgaagggcccactttagcaaaggccgctgccttcactccaggctggctctgaatgaagagtgtggggtggagcttccttgggatgacccggtggcctcaccccccatggtgggcttcttgtgctcctctctgggtgtccgctgcactccagttcttgcttcacctgggggagcaactggcgggctgctctttggccctctttgctatggtcatcattttttccagaccatggttctattttgccttcccagcgtcaggcctaagcatcctgatgtggaccagccttcctgggaccacttgcttcagaggcataggggaggctggtcaggctctgggaatgcttgtagacaggtttgttcacttcagaacttggcagcgtctcaattagatggccagcaacacagacctatgcattgccttctatgaatgtgccgttatgcaggcagctgtgtaattggatgggttttttttttttttttttttttaaagacattttaatgcacccactatcttcagcaggctttgttgtgttaagtctcagcacatgttggatgggtggccatccagcggacatctccttcctgttgtttcagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtcc

SEQ ID NO: 4 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 5 - DNA, 8651 bp, Homo sapiens
tgtatatcagggccgcgctgagctgcgccagctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggtgaatgacgggcgtgggtcggtgcgcgctcggcttgcgcacacggtgtctctaagtgcgcgggtgacgagagtcgggatgtgccggagaccccggggcggagagcgggattacaagtacaggaatccctggtcacgctccccgcccctggaaacccagctggggcgagggagggcgtggacgggaccgttctgggagctcgcctttggctgcggttggctccaggccccaggcgcagtttgctcgcggcgtggggatgaagtccgtgtccctggaggggcccaggaagggcgaggaaagcggagtggagtaagttcgtctaggatcggtcccgggtggctctgggatccaatctgcgccgccctggcccaggtcccaggttcaggtcctttacgccactgtgtccaccacctggctgagcgctgaggtcagcgcgggctgtttcctggcccttgggaatgtgccaggacccgtcccctaaggactagcgaggaggtgactcactgtgacaaggagaccccagggaacggactgtatgaggtcagaaccccgcccgggatggggtacagcgggactccagaagccctctcccctgccccttcgcggtctccgtcctcccatcggcacagtgacctatttggctggaacagtttgttcccaaggaagccgggcactggaggtccgggacaccgcgtcgggtccccgctccgcggcgcgctgtaggggtcggggagtcacggccctgctctgggcgggctctaaccagcctgtcagtcggggaagggcaagggtctcctctacctctttcccaccgcggccgggagaatcgcggcccagcctgtcctcgggtcggggcgctggactccggggcgggagcggagcccacgcctggatgggaggcggggagggttcatgtctttgaggggtggggggtctggggggcacgacgctgctcagggcctctatcagctgcctcgggggctcagggcttcccgacctagcccagattccctctccgaaagctacagggctgagcggagcaggggggcgagtcgccccctggggcgccgccgcctggcgcggaccacagcgcgtcctctccgtcccaaacccctgggggacacttgcgccctcttcgtgaggaaaagcatcttggagctgggttaggaacttggggcgcccaggcagcttcccctctccttgcctccctccacgtcgcgtttctgggaggacttgcgagcggttttgttttcgttgctcccgtctatttttattttccagggatctgactcatcccgtgctttgggcgtggagataaggtggaggggccggctcccggcgcgcgcgcgcgtgcgtgtctgcgcgggcgtgtgtgtgtgtgtgtgtgtgtgtgtgtgtgtgtgtgtctgtgtcagagacggcacaagagcgcgcggtttcccaacagcggcgggagtttcggaagcctggccggctcagcgtgacgtgttcgcggccccccggtcccctcccattctccccctccccaccccagggtgacgcgcagccggagtggaagcagttttggcgggcgagcagcgccttgcaggaaactgactcatcactactccctccagcggtccgaggctctgcccacgcacctcccactccgcgcgtgatttcctggaggccggcgccccctcccggccctggcgggaatagcacacaggctttcccgcggagtggggctggccggcgcgaaccgccgcggctactcctgggctcatccgagatcaacccctatgccattaccaccccttcaaaggagcactccttaggttcaacagtattcactgagctcttactggaaattaaaatatggctgaagtctaaggcaggaaggccaataaaggaggctatttttaattgtttctaaaacaagggtttgcgtttctgagttttctttgggctgaaagttattatgagcatgagagcagattttgatgggggaggagaggcctatgagagccataagagaaggaggggtggtagaagaggagagggtgcctgcctagatcctagtcctgtcttgaactcccgagagccagggaatatccagcaccttgatgaagccctaggcgggcgcctcctccttgtgcctatgatgtattgagacccagaatgtccatttcaaacataccagtgtgtctccgcttggctggcaacccaagagtgcccatctgaggaattgtgccaaacacttgcttgaatcttcaatctggattaagttggtctcgggaggcagggcctcagcaatctatattttgaaaaaactccctaggtgcttttctttctttctttttttctttctttctttctttctttctttctttctttctttctttctttctttctttttctttctttctttctttttctttcttttctttctttctttctttctttctttctttctttctttctttcttcctttctctttctctctttctttctctttctctctttctttcttttctttcgacagagttgcactctgtcacccaggctggagtgcaatggcaccatcctggactcaagtagtcctcctgtttcagcctcccaagtaaccgggaccacaggcgtgatccccccgcccccatgcccagatttttttttttttttttttttttttgagatgcggtctcgctctgtcacccaggctggagtgcagtggcgtgatctcggctcactgcaagctccgcctcccgggttcacgccattctcctgcctcagcctcccgagtagctgggactacaggtgctgccatcatgcccggctaatttttttttgtatttttagtagagacggggtttcaccgtgttagccaggatggtctcaatctcctgacctcgtgctccgccctcctcggcctcccaaagtgctgggattacaggtgtgagacactgcacccaaccacccagctaatttttatttatttttatttttagtagagacagggtctcagctagttgcccaggctagtcttggacccttgggctcaaatgattctcccacctctgcctcccagagtattaggattacaggcataagccactgcccctggcctccccaagtgattgtgatgggcctctctggttaagaaacctcaaaattagagagggagtggggttcaatactacagcacaggactcagggcaaacaggcctgggttcagatcctggctgtgccacttatgaactgtgtgatgttaggcaagttacttaacttatctgagccttggttgcctcttctgtaaaaagggagctaatagatatccactttttaggaggattgatatttttaaactgcttagaacagcccccaaacataaaaatatataaataaatcccaactcatgcctagcagagggtggatagaggttatttgagggctctgtccactgtactgggtgacccctttatggggcagtggcctttggcctttttagctgtatgactcaggggcaagtctcatatctcttccatctcctgccctttaaacttggtgtgaagttaccaagagcctcctctcccaaccagctgggacgtgaaactgtgggctccactgatcacaagcagtggggtgaggtggggtggagcagatgtggcatgtgtcccgggcttcctgcctcatgaggactcagcagagctttcacccccagaaactgcaagttgggacttgtccctaggaaaatccagttgctgccaaggtcgtgcagtcactcagccctggagtcaagccagagcaggcaggtaggtgccagggctccctcatgggcaaactcactctccgttttccctctcctgaagggggaggagaggagccaggtagaccagccacctttaattttctttttgcctgcaaaacggtttccttggacacaggcaacacgaggcaggggctgccaggtgtctagacttcagatcacctgatgtgcctggcaggatgtggctcagcctgggagaaatcatcccttgcgctgccccgcccggcccctccttacccctaggccacccgcctgacgacatccttgggaaaggccctcagcctacagcacctgtcagctgctgtctgaaggaggtagttggcagggggaagtgataggggggaggctcagtaaaactgaaggcagagaggaataatcatacttctgttttcaatgcacttctctatacgaagtgctgctggcacgttacctacattaactcagttaattctcatgtctatcctctgagacagtcactattactatccccattttatagatgaggaaactagagctcagacaagttaagttgcttgcccagggtcacctagtaaaacctggactccagcccaggtgatctggctccagagccctcctgcttaaccaccaggatacagcctttcattcagctctgttctgtctgccttgctgcatggactctgtgatcaatttcttgagtatgtgtctgtagccatgctctttaaacttgtacatggccccatttatggatgaggaaactgagacctagagacattaagtggctttttaaagcttacgtagtaactggcagagctaggaccacaacccgggtgctttttgccccaaagtcccgggtacttttacttggcagagcagggttaccctacttggggatctgggtcgggggacttaggaggctggaggaactgtcagactgtttcttcttttgggaattgaccttctggccagggctgcgattaggaaactgctggactctggcaattcacacatatttggggggcattcacacccatgagggacacctctggggggaaaacaaattgattttagctgataatacctggtggcaaacaggaccctggtccttgctcttgcaatagacttgcctttgttgacattagcttgcccttcagttgcctgctctcccagtgaccttggtgtgccaggctggctgagctctgctggtgggggtcaggcctcctgtgggaaggaagcaggaagaccagctggaaggagtgagagagaccctctggtaggaagacgtcacctgaggtgacacagcaaagcccggccaggtaacatagtgtctaatctccgccgtgaccagggccttccttgtatctctgctgcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacaggtgcggacatgtgcacggaaggactttgtaagggaccaggattctcagaatccatggtccaagggctgacctgtctggtcctggtccagcatgctccaggtagaaggaaacaggcccagagaggggaagcaacctccctgaggtcacacagcaagtaggcagcaaagaccaactagctaacatttattgggaatgttcattatgccaggccctttgccaagcttctaaggtagatttatttagtccttatagcaatgttataacataagacattcttgtcaccctgcccgcctttctttttgagacaggtgtcttaactctgttggccagactggagtgcagtgatacgatcatggctcactgcagcttcaaactcctgggctcaagcgatcttcctacctcagcctcctgggtagctgggaagctgggactatagttgtacaccactacgcccggttaattttttgagtttttgtagagacaaggtctcaccatgttgcccgggctggtcttgaactcctgagctcaagcagtcctcctgcctcagcctcccaaagtgttgtgattacaggcgtgagccaccatgcccagccccttgccatccttttagggcaaggaaaccaggctcagagaggtagagtgatttatctaaggtctcaaagtgaatttgccgttgggtcaagactaattataataacaacaactactgacgtttatatgggcccggcattgtgctgaacactttcatggattttgtaacagaatccctagatcagcactgtccagtaactctgcagggatgggagtgtccggtacaggggccacgagccacatacggctgttgtgcatttgacacacagctcatgtgactgaggaactgaattgttcattttatttgattgtagtctgtttaaacaagcacacagagctagtagtggttcctctgctgggcagcttgacttagagcagacccatgggtgcgggtgcggtgatggataaaatcacatctgtgaagcatggtgggacactccataatacccctcaagagacagagtggacgttccccgagttcttcctgttctcagcagtcggccccattggccccagggaagggtgtcctggccccccactgtcttcctcagttgggcagctccgccgcgtcctcttcttcttggcctggctgacttctgctgtctctcctcagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctaccctttttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgacttt

SEQ ID NO: 6 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 7 - protein, 114 aa, Artificial Sequence
  sequence of the SET domain of EZH2 at Location 618 - 731 of SEQ ID NO. 4
HLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDY

SEQ ID NO: 8 - DNA, 75 bp, Artificial Sequence
  siRNA to EZH2
ugcccuggcucaguuaucacagugcugaugcugucuauucuaaagguacaguacugugauaacugaaggauggca

SEQ ID NO: 9 - DNA, 19 bp, Artificial Sequence
  64-82bp in mRNA sequence
cggugggacucagaaggca

SEQ ID NO: 10 - RNA, 19 nt, Artificial Sequence
  siRNA to knockdown EZH2 protein activity
gacucugaaugcaguugcu

SEQ ID NO: 11 - RNA, 19 nt, Artificial Sequence
  siRNA that knocks down FBXO32
gucacauccuuuccuggaa

SEQ ID NO: 12 - RNA, 21 nt, Artificial Sequence
  FBXO32 shRNA vector insert
cagaagauuauauggcgcgaa